(19) United States
(12) Patent Application Publication
(10) Pub. No.: US 2023/0256099 A1
(43) Pub. Date: Aug. 17, 2023

(54) Safe Use of Bile Acids and Their Salts as Enhancers for Nasal Delivery of Pharmaceuticals

(71) Applicant: Amphastar Pharmaceuticals, Inc., Rancho Cucamonga, CA (US)

(72) Inventors: Jack Yongfeng Zhang, Rancho Cucamonga, CA (US); Mary Zi-ping Lou, Rancho Cucamonga, CA (US); Jie Fei Ding, Rancho Cucamonga, CA (US); Aili Bo, Rancho Cucamonga, CA (US)

(21) Appl. No.: 17/917,455

(22) PCT Filed: May 3, 2021
(86) PCT No.: PCT/US21/30504

§ 371 Date: Oct. 6, 2022

Related U.S. Application Data
(60) Provisional application No. 63/020,000, filed on May 4, 2020.

(51) Int. Cl. A61K 47/28; A61K 9/00

(52) U.S. Cl. CPC A61K 47/28; A61K 9/0073

(57) ABSTRACT

The present disclosure introduces safe and effective pharmaceutical formulations for intranasal delivery. Specifically, the present disclosure introduces the safe clinical use of bile acids or salts thereof as an enhancer to exhibit improved bioavailability and tissue tolerance. In several embodiments, pharmaceutical formulations including bile acids or salts thereof are provided. In several embodiments, the formulations are suitable and/or configured for the intranasal (IN) delivery, methods of manufacturing such formulations, and methods of treating patients using such formulations. The pharmaceutical formulations comprising bile acids, salts of bile acids, and/or combinations thereof. In several embodiments, bile acids, salts of bile acids, and/or combinations thereof are configured for use as absorption enhancers.

### CROSS REFERENCE TO RELATED APPLICATIONS

The present application claims the benefit of priority to U.S. Provisional Patent Application No. 63/020,000, filed May 4, 2020, the entirety of which is hereby incorporated by reference herein.

### FIELD

The present disclosure generally pertains to safe and effective pharmaceutical formulations for intranasal delivery. Specifically, the present disclosure introduces the safe clinical use of bile acids or salts thereof as an enhancer to exhibit improved bioavailability and tissue tolerance. In several embodiments, pharmaceutical formulations including bile acids or salts thereof are provided. In several embodiments, the formulations are suitable and/or configured for the intranasal (IN) delivery, methods of manufacturing such formulations, and methods of treating patients using such formulations.

### BACKGROUND

The advancement of technology has brought in different routes of administration to delivery different therapeutic agents. Currently, many drugs are administered to a human patient by injection using a needle (e.g. auto-injector, pre-filled syringe, injectables) to get the drug, and specifically the active pharmaceutical ingredient, or API, into the bloodstream quicker and avoid degradation of the drug by oral consumption. Bioavailability is the extent to which drug absorption occurs. While drug absorption is the movement of a drug from the site of drug administration to systemic circulation, bioavailability is the fraction of the administered drug that reaches the systemic circulation in the unchanged form. Drug bioavailability is affected by various factors, including physicochemical properties of the drug, physiological aspects, type of dosage form, biorhythms, and intra- and inter-individual variability of the human population. Many therapeutic agents are being developed for non-invasive administration, such as via nasal administration. However, many API have poor absorption when administered using non-invasive methods, making these non-invasive routes of administration impractical for an effective drug delivery.

### SUMMARY

The present disclosure solves one or more technical challenges and introduces safe and effective pharmaceutical formulations utilizing bile acids or salts thereof as absorption enhancers to facilitate the absorption of APIs via IN delivery. Advantageously, the present disclosure demonstrates a safe use of the bile acids or salts thereof, as an absorption enhancer via IN delivery for clinical use at concentrations above 3 mg/mL, which was reported to be clinically infeasible by industry. The present disclosure demonstrates that any damage or change to the mucosa caused by the bile acid or salts thereof is substantially reversible within 3-7 days, even at bile salt/acid concentrations above 3 mg/mL.

Surprisingly, bile acids and salts thereof can be used as effective enhancers for formulations of pharmaceutical medications with pharmaceutical efficacy and safety for human and animals for intranasal delivery with physiologically reversible damages. The disclosure herein demonstrates that, unexpectedly, no permanent damage occurs when administering bile acids and/or the salts thereof (e.g., in the formulations disclosed herein), allowing recovery times correlated with the dosing frequency. It was also noted that bile acids and the salts thereof improve the bioavailability and enable nasal delivery of medication in a comparable way of other routes of administration (including such as that of intramuscular (or IM)). It was surprisingly noted that irreversible damage to the mucosa does not occur if the concentration of bile salts is less than 1.5% based on the total weight of the formulations. This finding was unexpected and surprisingly considering that prior research had taught concentrations as low as about 0.3% caused irreversible damage to the nasal passages. As an enhancer and in the formulations disclosed herein, bile acids and salts thereof are able to improve absorption of small drug molecules and biologic, complex molecules in the pharmaceutical medications. Absorption enhancement from bile acids and the salts thereof make it possible for non-invasive delivery of some pharmaceutical medications, including intranasal and pulmonary delivery.

As is noted elsewhere herein, historically, bile acids and the salts thereof have not been successfully implemented for clinical use in human subjects due to various toxicity issues. In several embodiments disclosed herein, surprisingly safe and effective pharmaceutical formulations are disclosed using a bile acid or salt thereof bile salt (e.g., a bile acid salt such as sodium taurocholate or STC) as an enhancer for intranasal administration. First, a set of formulations was selected to evaluate the change of absorption ratio with various STC concentrations. The STC concentrations in the formulations were 0-10 mg/mL. The formulations were evaluated in a randomized, active-controlled, evaluator-blinded, crossover study in 56 healthy volunteers (male and female between 18 and 50 years old). It was found that STC significantly increased the absorption of API as the concentration of STC increased. For example, for Epinephrine API, in general, the added STC should reach to an appreciable level to play the role of an enhancer. The relative bioavailability (RBA; defined as RBA=Parameter (IN)*Dose (IM)/Parameter (IM)*Dose (IN)) of IN delivery, which increases from almost 0% without STC to 34.6% with STC. Surprisingly, the formulations disclosed herein were well-tolerated by the patients.

As disclosed elsewhere herein, poor absorption of a drug is a major concern. In certain circumstances, the lack of absorbability makes it impractical for an effective drug delivery. It is also well known that the absorption of for example, epinephrine on its own is so poor that it is prohibitive for intranasal delivery. This is consistent with the clinical study data disclosed herein showing that epinephrine without addition of STC exhibit a relative bioavailability (RBA) of almost 0%. The results disclosed herein demonstrate that IN delivery with STC could reach to a comparable level of drug absorption (Cₘₐₓ, AUC, and tₘₐₓ) with that of the traditional IM route of administration. When a fast onset of effect is required, IN with STC might be more advantageous with respect to IM.

In aqueous solutions, bile salts aggregate and form micelles in concentrations above critical micelle concentration (CMC). By forming micelles, bile salts can facilitate transcellular passage and enhance absorption. Further, the clinical study data demonstrated that the enhancement of absorption by STC bile salt increased significantly at a concentration of greater than 6 mg/mL or preferably greater than 8 mg/mL, with the absorption enhancement of about 3 and 5 folded, respectively.

However, bile salts or acids had little clinical use due to safety concerns with respect to irreversible alteration of the epithelial cell of mucosa membrane. This became the consensus in both academic and industry settings. This invention however, demonstrates that if STC's concentration is less than 15 mg/mL, the physiological damage is reversible.

Local irritation was assessed by Nasal and Oropharyngeal Mucosa Examination (NOME) evaluated by ENT specialist or qualified physicians; Subjects Self-Reported Nasal Symptoms (SRNS); and University of Pennsylvania Smell Identification Test (UPSIT). The evaluated scores of both physicians and patients demonstrated Epinephrine/STC caused mild to moderate nasal irritation at turbinate or nasal discomfort in the STC concentration of 6-10 mg/mL. However, the rate for severe local irritation caused by Epinephrine/STC is very low. No impact of epinephrine formulation/STC on smell function was observed. The reported local irritation is recoverable per the SRNS and ADE data. The irritations recovered to baseline in about 2 weeks at the follow-up visit, which allows a full compliance with the dosing frequency generally required.

The six (6) major adverse drug events, or ADEs, reported in the clinical study with Epinephrine/STC are divided into 3 groups: ADEs related to vital signs, including tachypnea, cardiac disorder and vascular disorder, which occurred in a similar rate with the intramuscular treatment of epinephrine and are considered irrelevant to STC; ADEs related to IN delivery including nasal edema/erosion and other nasal ADE; and other ADEs, such as headache, which did not show obvious treatment-response effect.

Surprisingly, the formulations disclosed herein are safe and effective, utilizing bile acids or salts thereof, as an absorption enhancer to facilitate the absorption of APIs via IN delivery where irreversible damage to the mucosa will not occur if the concentration of bile salts or acids is less than 15 mg/mL.

The API could be small molecules, which are typically comprised of 20 to 100 atoms and have a molecular mass of less than 1000 g/mol or 1 kilodalton [kDa]; complex molecules; or biologic molecules, which includes proteins, peptides, and nucleic acid-based agents that typically contain from 5,000 to 50,000 atoms per molecule.

As an enhancer, bile salts improve absorption of APIs. These APIs in general have low permeability and exhibit many characteristics that are problematic for effective drug delivery. Some of the problems associated include poor permeability, erratic and poor absorption, inter- and intra-subject variability and significant food effects, which lead to low and variable bioavailability.

Several embodiments disclose bile salts as an excipient added to improve the IN absorption of small molecules (e.g., epinephrine and naloxone). Further, bile salts also can improve the IN absorption of biologics, complex molecules, such as insulin aspart. As described herein, STC significantly improves the efficacy of the API absorptions and safety is demonstrated by histopathological data. Absorption enhancement of bile salts makes it possible for non-invasive delivery of some pharmaceutical medications, including by intranasal and pulmonary delivery.

Several embodiments pertain to a pharmaceutical formulation. In several embodiments, the pharmaceutical formulation comprises a therapeutically effective amount of an active pharmaceutical ingredient (API). In several embodiments, the pharmaceutical formulation comprises an absorption enhancer. In several embodiments, the absorption enhancer comprises, consists essentially of, or consists of one or more bile acids or bile acid salts at a concentration that is greater than 3 mg/ml. In several embodiments, the pharmaceutical formulation is provided as a liquid (e.g., a solution) or a dry powder. In several embodiments, the pharmaceutical formulation comprises an aqueous carrier. In several embodiments, the pharmaceutical formulation is configured to be administered intranasally and/or via an intrapulmonary route. In several embodiments, the pharmaceutical formulation is safe and effective for use in a subject, causing no irreversible damage to the subject.

In several embodiments, irritation or adverse effects caused by administration of the pharmaceutical formulation are transient. In several embodiments, irritation or adverse effects caused by administration of the pharmaceutical formulation resolve in less than or equal to 1 day, three days, one week, or two weeks. In several embodiments, the concentration of one or more bile acids or bile acid salts is provided at a concentration of equal to or greater than 1.5 weight percent in the formulation.

In several embodiments, the one or more bile acids or bile acid salts are provided at a concentration above its critical micelle concentration (CMC). In several embodiments, the formulation comprises micelles that include the one or more bile acids or bile acid salts. In several embodiments, the micelles are configured to facilitate transcellular passage and enhance absorption of the API.

In several embodiments, the API is a small drug molecules or a large biologic and/or complex molecules.

In several embodiments, the absorption enhancer is configured to provide bioavailability of the API that is comparable to administration of the API through an intramuscular route and/or wherein intranasal administration using the formulation may be used as a substitute for the intramuscular route.

In several embodiments, the formulation comprises a therapeutically effective amount of API is suitable for the treatment of a type-I hypersensitivity reaction.

In several embodiments, the absorption enhancer comprises, consists essentially of, or consists of a taurocholate salt. In several embodiments, the absorption enhancer comprises, consists essentially of, or consists of sodium taurocholate. In several embodiments, the absorption enhancer comprises, consists essentially of, or consists of a taurochenodeoxycholate. In several embodiments, the absorption enhancer comprises, consists essentially of, or consists of sodium taurochenodeoxycholate.

In several embodiments, the pharmaceutical formulation further comprises a buffer.

In several embodiments, the pharmaceutical formulation further comprises a preservative.

In several embodiments, the pharmaceutical formulation further comprises a tonicity agent.

In several embodiments, the pharmaceutical formulation further comprises a metal complexing agent.

In several embodiments, the pharmaceutical formulation further comprises an antioxidant.

In several embodiments, the pharmaceutical formulation has an osmolarity ranging from 200 mOsmol to 260 mOsmol.

In several embodiments, a dose delivered to a nasal mucosa of a human subject provides a tₘₐₓ of equal to or less than 10 minutes. In several embodiments, a dose of the pharmaceutical is less than or equal to 0.1 mL. In several embodiments, the pharmaceutical formulation is configured to be delivered as an atomized spray.

In several embodiments, no grade 2 or 3 events occur in the subject after a nasal and oropharyngeal mucosa examination (NOME). In several embodiments, no grade 3 events occur in the subject under self-reported nasal symptoms (SRNS) testing. In several embodiments, the subject experiences the same or improved normosmia after administration of the formulation to the nose as measured by the University of Pennsylvania Smell Identification Test (UPSIT).

Several embodiments pertain to a method of treating a condition in a patient. In several embodiments, the method comprises administering an intranasal dose of the pharmaceutical formulation as disclosed elsewhere herein to at least a nostril of a human patient to treat a condition.

Several embodiments pertain to a pharmaceutical formulation as disclosed elsewhere herein for use in treating a condition of a patient.

Several embodiments pertain to a method of preparing a pharmaceutical formulation. In several embodiments, the method comprises dissolving API or a pharmaceutically acceptable salt thereof and an absorption enhancer in water. In several embodiments, the absorption enhancer consists of a bile acid or bile acid salt. In several embodiments, a final concentration of the absorption enhancer in the pharmaceutical formulation ranges from 1.0 mg/ml to 15 mg/ml. In several embodiments, the pharmaceutical formulation is configured to be administered intranasally.

Several embodiments pertain to a pharmaceutical formulation for intranasal (IN) delivery. In several embodiments, the formulation comprises an active pharmaceutical ingredient (API). In several embodiments, the formulation comprises an absorption enhancer comprising a bile acid, or a salt thereof. In several embodiments, the bile acid, of the salt thereof, enhances absorption of the API by IN delivery in a human subject.

In several embodiments, the bile acid, or the salt thereof, is present at a concentration of at a concentration of at least 3.0 mg/mL. In several embodiments, the bile acid, or the salt thereof, is present at a concentration of 3.0 mg/mL to 15.0 mg/mL. In several embodiments, the bile acid, or the salt thereof, is present at a concentration of 5.0 mg/mL to 13.0 mg/mL.

In several embodiments, the delivery volume of the formulation is in the range 0.05 to 0.25 mL. In several embodiments, the bile acid, or the salt thereof, is present at a dose amount of at least 0.15 mg. In several embodiments, the bile acid, or the salt thereof, is present at a dose amount of at least 0.15 mg to 3.8 mg. In several embodiments, the bile acid, or the salt thereof, is present at a dose amount of at least 0.25 mg to 3.1 mg.

In several embodiments, if the bile acid, or the salt thereof, causes decreased cilia in a respiratory epithelium of the human subject, then such decreased cilia is substantially reversed within 7 days. In several embodiments, if the bile acid, or the salt thereof, causes hyperplasia of a respiratory epithelium of the human subject, then such hyperplasia is substantially reversed within 7 days.

In several embodiments, the bile acid, or the salt thereof, comprises a trihydroxy conjugate. In some embodiments, the bile acid is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), or a combination thereof. In several embodiments, the bile salt is a trihydroxy conjugate comprising sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), or a combination thereof. Other suitable forms of salts are possible, such as substituting sodium with potassium (e.g. potassium glycocholate), or a combination thereof.

In several embodiments, the bile acid, or the salt thereof, is a dihydroxy conjugate. In several embodiments, the bile salt is a dihydroxy conjugate comprising comprises sodium tauroursodeoxycholate (STUDC), sodium taurohyodeoxycholate (STHDC), sodium glycohyodeoxycholate (SGHDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination thereof. In several embodiments, the bile acid is a dihydroxy conjugate comprising tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination thereof.

In several embodiments, the bile acid, or the salt thereof, is an unconjugated form. In several embodiments, the bile acid is an unconjugated form comprising cholate, deoxycholate (DC), chenodeoxycholate (CDC), or a combination thereof.

In several embodiments, the bile salt is an unconjugated form comprising sodium cholate (SC), sodium deoxycholate (SDC), sodium chenodeoxycholate (SCDC), or a combination thereof.

In several embodiments, the bile salt is STC. In several embodiments, the bile salt is STCDC.

In several embodiments, the API is a small molecule having a molecular weight of less than 900 g/mol. In several embodiments, the API is a small molecule comprising an adrenergic agonist. In several embodiments, the API is a small molecule comprising an adrenergic agonist, wherein the adrenergic agonist includes epinephrine, norepinephrine, dopamine, isoprenaline, phenylephrine, dexmedetomidine, oxymetazoline, methyldopa, clonidine, dobutamine, salbutamol, albuterol, terbutaline, salmeterol, formoterol, or pirbuterol. In several embodiments, the API is a small molecule comprising an opioid antagonist. In several embodiments, the API is a small molecule comprising an opioid antagonist, the opioid antagonist includes naloxone, nalmefene, and/or naltrexone.

In several embodiments, the API is a large molecule having a molecular weight of 900 g/mol or more. In several embodiments, the API is a large molecule comprising a protein. In several embodiments, the API is a large molecule comprising a protein, wherein the protein includes insulin, insulin aspart, or insulin glargine.

In several embodiments, the bile acid, or the salt thereof, provides an Enhancement Factor (EF) of at least 4, wherein the EF is determined based on R̅(S)/R̅(0), where R̅(S) is an average of a dose-normalized relative bioavailability (DN-RBA) of the pharmaceutical formulation relative to an IM injection having same API for two or more pharmacokinetic (PK) parameters, and R̅(0) is an average of DN-RBA of a pharmaceutical formulation without an absorption enhancer relative to an IM injection having same API for the two or more PK parameters. In several embodiments, the EF is in a range of 4 to 23. In several embodiments, the PK parameters include AUC_{0-30 min}, AUC_{0-180 min}, and Cₘₐₓ.

Several embodiments pertain to a method of delivering an active pharmaceutical ingredient. In several embodiments, the method comprises administering a pharmaceutical formulation to a human subject by intranasal (IN) delivery using a nasal spray. In several embodiments, the pharmaceutical formulation comprises a therapeutically effective amount of the active pharmaceutical ingredient (API) and an absorption enhancer comprising a bile acid, or a salt thereof. In several embodiments, the bile acid, or the salt thereof, enhances absorption of the API by IN delivery in the human subject. In several embodiments, the bile acid, or the salt thereof, is present at a concentration of at least 3.0 mg/mL. In several embodiments, the bile acid, or the salt thereof, is present at a concentration of 3.0 mg/mL to 15.0 mg/mL. In several embodiments, the bile acid, or the salt thereof, is present at a concentration of 5.0 mg/mL to 13.0 mg/mL. In several embodiments, the dose volume is discharged in one spray of the nasal spray. In several embodiments, the bile acid, or the salt thereof, is present at a dose amount of at least 0.3 mg. In several embodiments, the bile acid, or the salt thereof, is present at a dose amount of at least 0.3 mg to 1.5 mg. In several embodiments, the bile acid, or the salt thereof, is present at a dose amount of at least 0.5 mg to 1.3 mg. In several embodiments, if the bile acid, or the salt thereof, causes decreased cilia in a respiratory epithelium of the human subject, then such decreased cilia is reversible within 7 days. In several embodiments, if the bile acid, or the salt thereof, causes hyperplasia of a respiratory epithelium of the human subject, then such hyperplasia is reversible within 7 days. In several embodiments, the bile acid, or the salt thereof, comprises a trihydroxy conjugate.

In several embodiments, the bile acid, or the salt thereof, provides an EF of at least 4, wherein the EF is determined based on R̅(S)/R̅(0), where R̅(S) is an average of a dose-normalized relative bioavailability (DN-RBA) of the pharmaceutical formulation relative to an IM injection having same API for two or more pharmacokinetic (PK) parameters, and R̅(0) is an average of DN-RBA of a pharmaceutical formulation without an absorption enhancer relative to an IM injection having same API for the two or more PK parameters. In several embodiments, the EF is in a range of 4 to 23. In several embodiments, the PK parameters include AUC_{0-30 min}, AUC_{0-180 min}, and Cₘₐₓ.

### BRIEF DESCRIPTION OF THE DRAWINGS

Exemplary features and advantages of certain exemplary embodiments of the present disclosure will become more apparent from the following description of certain exemplary embodiments thereof when taken in conjunction with the accompanying drawings.

FIG. 1 is a graph illustrating the Bile Salt (STC) Enhancement Effect on IN formulations based on Bile Salt (STC) Enhancement Factor versus Bile Salt (STC) concentration utilizing sodium taurocholate (STC) as the bile salt, which is further detailed in the Examples.

FIG. 2 is a graph illustrating the local irritation of the nasal mucosa by Bile Salt (STC) and in particular, the average total irritation points (TIP) for the findings in the Examples versus time after IN treatment.

FIG. 3 is a graph illustrating the local irritation of the nasal mucosa by Bile Salt (STC) and in particular, the average M^{3,4} for the findings in the Examples versus time after IN treatment.

FIG. 4A is a graph illustrating the local irritation of the nasal mucosa by Bile Salt (STC) and in particular, the TIP for erosion/flattening in the Examples versus time after IN treatment.

FIG. 4B is a graph illustrating the local irritation of the nasal mucosa by Bile Salt (STC) and in particular, the TIP for decreased cilia in the Examples versus time after IN treatment.

FIG. 4C is a graph illustrating the local irritation of the nasal mucosa by Bile Salt (STC) and in particular, the TIP for hyperplasia in the Examples versus time after IN treatment.

FIG. 5A is a graph illustrating the mean naloxone concentration in rat serum from 0 min. to 180 min utilizing STC as the bile salt, which is further detailed in the Examples.

FIG. 5B is a graph illustrating the mean naloxone concentration in rat serum from 0 min. to 30 mins. utilizing STC as the bile salt, which is further detailed in the Examples.

FIG. 5C is a graph illustrating the mean naloxone concentration in rat serum by IN delivery utilizing STC as the bile salt, which is further detailed in the Examples.

FIG. 6A is a graph illustrating the relative bioavailability (RBA) of epinephrine, IN versus IM, using two exemplary bile salts STC and sodium taurochenodeoxycholate (STCDC), which is further detailed in the Examples.

FIG. 6B is a graph illustrating the mean epinephrine concentration in rat serum from 0 min. to 180 mins. utilizing STC as the bile salt, which is further detailed in the Examples.

FIG. 6C is a graph illustrating the mean epinephrine concentration in rat serum from 0 min. to 180 mins. utilizing STCDC as the bile salt, which is further detailed in the Examples.

FIG. 7A is a graph illustrating the average total observation point (TOP) of a histopathologic study of a rat's nasal mucosa, using STCDC as the exemplary bile salt and epinephrine as the exemplary API, which is further detailed in the Examples.

FIG. 7B is a graph illustrating the average occurrence of Level 3 (Moderate) of a histopathologic study of a rat's nasal mucosa, using STCDC as the exemplary bile salt and epinephrine as the exemplary API, which is further detailed in the Examples.

FIG. 7C is a graph illustrating the average occurrence of Level 4 (Marked) of a histopathologic study of a rat's nasal mucosa, using STCDC as the exemplary bile salt and epinephrine as the exemplary API, which is further detailed in the Examples.

FIG. 7D is a graph illustrating the average TOP of a histopathologic study of a rat's nasal mucosa, using STCDC as the exemplary bile salt and epinephrine as the exemplary API, which is further detailed in the Examples.

FIG. 8A is a graph illustrating the average occurrence levels (AOL) of a histopathologic study of a rat's nasal mucosa for Group 3 in Example 6, using STCDC as the exemplary bile salt and epinephrine as the exemplary API.

FIG. 8B is a graph illustrating the AOL of a histopathologic study of a rat's nasal mucosa for Group 4 in Example 6, using STCDC as the exemplary bile salt and epinephrine as the exemplary API.

FIG. 8C is a graph illustrating the AOL of a histopathologic study of a rat's nasal mucosa for Group 5 in Example 6, using STCDC as the exemplary bile salt and epinephrine as the exemplary API.

FIG. 8D is a graph illustrating the AOL of a histopathologic study of a rat's nasal mucosa for Group 6 in Example 6, using STCDC as the exemplary bile salt and epinephrine as the exemplary API.

FIGS. 9A-9D provide pharmacodynamic data for various embodiments of IN pharmaceutical formulations as disclosed herein versus an IM comparator formulation.

FIG. 10 provides relative total NOME Observation Rate per subject in a clinical study using humans.

FIGS. 11A and 11B provide information regarding the occurrence of adverse events during the clinical study.

FIG. 12A is a graph illustrating the mean insulin aspart concentration in rat plasma from 0 min. to 180 mins. administered by subcutaneous (SC) injection, which is further detailed in Example 8.

FIG. 12B is a graph illustrating the mean insulin aspart concentration in rat plasma from 0 min to 180 mins utilizing STC as the bile salt, and administered by IN administration, which is further detailed in Example 8.

Throughout the drawings, like reference numerals will be understood to refer to like elements, features and structures.

### DETAILED DESCRIPTION

The present disclosure generally pertains to safe and effective pharmaceutical formulations suitable for the nasal delivery (e.g., intranasal delivery). Specifically, the present disclosure introduces the safe clinical use of bile acids or salts thereof as an enhancer to exhibit improved bioavailability and tissue tolerance in human and animals. The present disclosure pertains to bile acids, or salts thereof, as absorption enhancer for intranasal (IN) delivery of an API into a human subject's bloodstream. Disclosed are pharmaceutical formulations and corresponding methods of use for IN delivery comprising an API and an absorption enhancer comprising a bile acid, or a salt thereof, wherein the absorption enhancer enhances the absorption of the API into a human subject's bloodstream in IN delivery. Several embodiments herein pertain to intranasal compositions (e.g., pharmaceutical formulations) comprising an API and a bile acid, methods for making or using such compositions, and/or methods of using such compositions. In several embodiments, the composition is useful in and/or is configured for intranasal delivery of an API. In several embodiments, the bile acid enhances the absorption of an API through the nasal mucosa of the nasal cavity. In several embodiments, the bile acid is provided as a bile acid salt. In several embodiments, the composition may further comprise one or more additional pharmaceutically acceptable carriers and/or one or more additional pharmaceutically acceptable excipients. The matters exemplified in this description are provided to assist in a comprehensive understanding of exemplary embodiments of the invention with reference to the accompanying drawings. While the present disclosure has been described in connection with certain embodiments, it is to be understood that the invention is not limited to the disclosed embodiments, but, to the contrary, the present disclosure is intended to cover various modifications and arrangements included within the spirit and scope of the appended claims, and equivalents thereof. Accordingly, those of ordinary skill in the art will recognize that various changes and modifications of the exemplary embodiments described herein can be made without departing from the scope and spirit of the claimed invention. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage or mode of operation.

Unless otherwise defined herein, scientific and technical terms used in connection with embodiments of present disclosure shall have the meanings that are commonly understood by those of ordinary skill in the art. Nomenclatures used in connection with, and techniques described herein are those known and commonly used in the art. Also, descriptions of well-known functions and constructions are omitted for clarity and conciseness.

“Pharmaceutical formulation” refers to a formulation comprising at least one active pharmaceutical ingredient (API) and at least one excipient (e.g., a bile acid or bile acid salt). For brevity herein, “IN pharmaceutical formulations” refers to pharmaceutical formulations configured for IN delivery. “IN API pharmaceutical formulations” (or similar language) refer to IN pharmaceutical formulations including at least an API, or a pharmaceutically acceptable salt thereof, for IN delivery.

The terms “active pharmaceutical ingredient” or “API” refers to one or more substances in a pharmaceutical formulation that is intended to provide the primary pharmacological effect. By contrast, an inactive pharmaceutical ingredient, such as an excipient in the pharmaceutical formulation, is not intended to provide the primary pharmacological effect.

The term “absorption enhancer,” as used herein in the context of IN delivery, refers to an excipient in the pharmaceutical formulation whose primary function is to modify, and preferably increase, the absorption of the API into a human subject's bloodstream (e.g., by enhancing permeation of the API through a nasal mucosa of the human subject).

“Pharmaceutically acceptable” refers to an ingredient in the pharmaceutical formulation that is compatible with the other ingredients in the formulation, and does not cause excess harm to the patient receiving the pharmaceutical formulation.

The term “pharmaceutically acceptable salt” refers to salts that retain the biological effectiveness and properties of a compound, which are not biologically or otherwise undesirable for use in a pharmaceutical. In many cases, the compounds herein (including bile acids) are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts (e.g., which form salts with bile acids) can be formed with inorganic and organic bases. Inorganic bases from which salts (e.g., bile salts) can be derived include, for example, sodium, potassium, lithium, ammonium, calcium, magnesium, iron, zinc, copper, manganese, aluminum, and the like; particularly ammonium, potassium, sodium, calcium and magnesium salts. Organic bases from which salts (e.g., bile salts) can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in U.S. Pat. No. 4,783,443A (incorporated by reference herein in its entirety).

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety. A carrier may be aqueous or may be water or saline (e.g., water, saline, saline for injection, etc.).

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

As used herein, the term “Cₘₐₓ” is given its plain and ordinary meaning and refers to the maximum (or peak) plasma concentration of an agent after it is administered. A Cₘₐₓ may be reported as the geometric and/or arithmetic mean of individual Cₘₐₓ values from a given patient population.

As used herein, the term “tₘₐₓ” is given its plain and ordinary meaning and refers to the length of time required for an active pharmaceutical ingredient or agent to reach maximum plasma concentration after the pharmaceutical composition, agent, or active pharmaceutical ingredient is administered. A tₘₐₓ may be reported as the geometric and/or arithmetic mean of individual tₘₐₓ values from a given patient population.

As used herein, the term “AUC” is given its plain and ordinary meaning and refers to the calculated area under the curve, referring to a plasma concentration-time curve (e.g., the definite integral in a plot of drug concentration in blood plasma vs. time). AUC may be reported as the geometric and/or arithmetic mean of individual AUC values from a given patient population. AUC may be reported as a partial AUC within a given time frame. For example, for the AUC between time points “a” and “b”, the AUC within that time window is reported as AUC, b. To illustrate, the AUC from time 0 (when the API is administered) to a time point 10 minutes later, to a time point 30 minutes later, to a time point 180 minutes later, to a time point 6 hours later, or a time where the blood concentration is less than the limit of detection, AUC is reported as AUC_{0-10 min}, AUC_{0-30 min}, AUC_{0-180 min}, AUC_{0-6 hr}, and AUC_{0-∞}, respectively. Other start points for AUC values may be taken by subtracting one AUC from another. For instance, an AUC_{30 min-6 hr} may be calculated by subtracting AUC_{0-30 min} from AUC_{0-6 hr} Other AUC values may be similarly calculated (e.g., AUC_{10 min-30 min}, AUC_{10 min-180 min}, AUC_{30 min-180 min}, AUC_{10 min-6 hr}, AUC_{180 min-180 min}, AUC_{10 min-∞}, AUC_{30 min-∞}, AUC_{180 min-∞}, and AUC_{6 hr-∞}).

When referring to the amount present for one or more ingredients, the terms “or ranges including and/or spanning the aforementioned values” (and variations thereof) is meant to include any range that includes or spans the aforementioned values. For example, when the concentration of an ingredient is expressed as 1 mg/mL, 5 mg/mL, 10 mg/mL, 20 mg/mL, “or ranges including and/or spanning the aforementioned values,” this includes wt % ranges for the ingredient spanning from 1 mg/mL to 20 mg/mL, 1 mg/mL to 10 mg/mL, 1 mg/mL to 5 mg/mL, 5 mg/mL to 20 mg/mL, 5 mg/mL to 10 mg/mL, and 10 mg/mL to 20 mg/mL.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “or” should not be read as requiring mutual exclusivity among that group, but rather should be read as “and/or” unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used together.

### INTRODUCTION

A number of APIs are configured to be delivered through ingestion or intramuscular (IM) delivery. In some instances, the gastrointestinal track can degrade an API and/or may not result in effective absorption. When a patient needs to use injectables, patients may be unable to comply with the required dosing specifications. Several use-related injuries have also been reported including unintentional injection, and lacerations (e.g. thigh lacerations) in healthcare providers and patients. On a practical level, patients may be reluctant to self-inject because of a general (or extreme) fear of needles, bleeding, pain/discomfort of a needle puncture, bruising, fear of needing multiple attempts to properly self-inject, anxiety, and inability to self-inject properly in emergency situations when the patient may not be calm or composed. Repeated training may be needed for a patient to learn how to properly self-inject.

In emergency situations, patients may not be in a calm or composed state of mind to properly self-inject emergency drug. For example, proper IM self-injection requires the patient to know and remember the optimal sites on the human body (e.g., thigh muscle) to inject the medication in order for the drug to be effectively absorbed into the bloodstream. As opposed to the upper arm, the thigh muscle is one of the body's largest muscles with more blood supply, so it allows much faster absorption of the medication. The outer thigh, versus the front of the thigh, is recommended because it provides a skin area with thinner tissue and less fat. Otherwise, if the patient injects the IM drug at non-optimal sites of the human body, then it may take a longer period of time for the drug to be absorbed into the bloodstream, which may be counter-productive in an emergency treatment. In addition, improper self-injection may lead to bleeding, swelling, numbness, tingling, lacerations, or other pain and discomfort.

A potential alternative to ingestion or injection delivery is to deliver the drug through the intranasal route of administration, also referred to as intranasal (IN) delivery herein. While some attempts to prepare compositions for intranasal administration have been attempted, these suffer from side effects, including pain in the nasal passages or other symptoms. Moreover, even where patients tolerate IN route, the compositions used often have pharmacokinetic (PK) profiles that are unlike those for the IM route and/or are disadvantageous. For instance, to achieve a desired Cₘₐₓ or tₘₐₓ, such as that provided by IM administration of the drug, the required dose of API (e.g., epinephrine) in an IN formulation may expose the patient to an unnecessarily high amount of the API overall (e.g., the AUC, such as the AUC_{0-∞}, may be unacceptably high or higher than needed). Where a desired AUC value is achieved (such an AUC comparable to that provided by IM administration), the Cₘₐₓ of API (e.g., epinephrine) may be too low or the tₘₐₓ too high, etc.

Absorption enhancers are excipients that may be included in formulations to modify, and preferably improve, the absorption of drugs across biological barriers. They have been investigated particularly to enhance the efficacy of pharmacologically active components that have poor absorption. The ideal absorption enhancer should be one that protects biological agents against enzymatic degradation and causes a rapid opening of the relevant barrier while transiently enhancing absorption.

As a platform for non-invasive delivery, nasal and pulmonary administration have several advantages over traditional oral medication or injection. Nasal and pulmonary delivery are non-invasive routes of administration that target the delivered dose directly to the site of drug action. Moreover, drug delivery to the respiratory area can also be used for systemic delivery of pharmacologically active components due to the large surface area (a highly vascularized mucous membrane) for drug absorption. As disclosed elsewhere herein, pulmonary and nasal administration also bypasses first-pass metabolism that is observed in oral administration and the lung and nasal cavity have a low drug metabolizing environment. However, despite all these advantages, pulmonary and nasal administration formulations remain elusive. Challenges remain significant in enhancing the absorption of the pharmacologically active components via these routes. For example, to date, no safe absorption enhancer for nasal and pulmonary administration of drugs has translated into a commercial product. Their use has generated safety concerns due to potential irreversible alteration of the epithelial cell membrane, which could potentially make the nasal cavity and lung susceptible to the entry of exogenous allergens.

Given the balance of requirements needed for effective administration of active pharmaceutical ingredients (APIs), there is an unmet medical need to develop drug products to overcome these disadvantages. Absorption enhancers that increase the rate of absorption are developed based on different mechanisms. In several embodiments, disclosed herein is the use of a bile acid or a pharmaceutically acceptable salt thereof as an enhancer to exhibit improved bioavailability and tissue tolerance. Several embodiments disclosed herein solve one or more of the above problems or others by providing pharmaceutical formulations suitable for intranasal and/or pulmonary delivery that are well-tolerated and have a desirable pharmacokinetic (PK) profiles. In several embodiments, using the IN formulations disclosed herein, surprisingly, desirable PK parameters, pharmacodynamic, safety, and/or tolerability profiles can be achieved through one or more of the intranasal and/or pulmonary routes.

For example, using bile acids (including bile acid salts as disclosed herein) as absorption enhancers, it has now been found that the intranasal route of administration can provide a rapid onset of drug action. In particular, using bile acids (and the salts thereof) the nasal cavity provides direct access to the bloodstream, thereby avoiding first-pass metabolism of portal circulation, and leading to a rapid onset of drug action. This is especially advantageous because the intranasal and/or pulmonary routes are a non-invasive drug delivery methods. By contrast, IM delivery requires the patient to inject deep into the muscle at the optimal sites, otherwise the drug may not be readily absorbed into the bloodstream. In addition, the compositions disclosed herein offer several advantages over IM, such as being easy to use, painless, easy to carry, and self-administrable without use of needles. A significant advantage is that these may be administered to children who do not know how to swallow pills, etc. Surprisingly, the compositions disclosed herein, which comprise bile acids (or salts thereof) as enhancing agents, achieve similar PK profiles to the IM route and ingestions routes or even potentially improved profiles.

As disclosed elsewhere herein, intranasal delivery utilizes drug absorption through the nasal cavity and more particularly, the nasal mucosa (also known as the respiratory mucosa), which is a highly vascularized mucous membrane that lines the nasal cavity. The nasal mucosa is made up of primarily two layers, an upper epithelial layer that is predominantly lipophilic, and a sub-layer known as the lamina propria. The upper epithelial layer is generally made up of epithelium, cilia, mucus (mucin), goblet cells (mucus-producing cells), and others cells. Notably, the lamina propria is highly vascularized with an extensive network of blood vessels, which can enable a drug to be rapidly absorbed into the bloodstream. However, in order to reach these blood vessels in the lamina propria, a pharmacological challenge is to develop pharmaceutical formulations that enhance drug absorption through the predominantly lipophilic upper epithelial layer.

The nasal cavity is the main passageway for air into and out of the lungs. Thus, a primary function of the nasal mucosa is to serve as an immune defense against foreign agents, such as drugs, allergens, pathogens, viruses, bacteria, dirt particles, and other airborne particulates. Consequently, it is a pharmacological challenge to achieve API absorption through the nasal mucosa and/or lungs. Additionally, many APIs, by themselves, have low membrane permeability, exacerbating these challenges. Thus, when aqueous API is delivered by the intranasal or pulmonary route, the absorption is very low. For example, the bioavailability (BA), based on the area under curve (AUC) in the plasma concentration of an API (e.g., epinephrine), may only be approximately 5% relative to that for the same dose of delivered by the IM route. For these reasons, epinephrine has historically been deemed to have limited therapeutic use by IN delivery. Therefore, an absorption enhancer for intranasal or pulmonary delivery strong enough to enhance the absorption of the API to a reach a BA similar to that achieved by IM route of the same API, but to do so without causing any significant damage to the body (e.g., the nasal mucosa).

Thus, an absorption enhancer is needed to enhance the absorption of the drug into the bloodstream via intranasal or pulmonary delivery, such as enhancing the absorption of an API into the network of blood vessels in the nasal mucosa. In this regard, another pharmacological challenge of IN delivery is to minimize or reducing the absorption enhancer's local toxicity to the nasal cavity. Minimizing local toxicity to the nasal cavity is important because the nasal mucosa provides a number of critical functions for the body (such as humidifying inhaled air, serving as an immune defense against foreign agents, etc.). Because of its role in the body, the nasal mucosa is also one of the most commonly infected tissues Inflammation of the nasal mucosa may cause a stuffy nose, headaches, mouth breathing, and other symptoms. This inflammation can be exacerbated by IN delivery of pharmaceutical agents, making it more challenging to develop drug formulations for IN administration.

Because of these challenges presented by the nasal cavity, there are currently very few drugs that are approved for IN delivery. To solve these challenges, the present disclosure introduces the use of bile acids, or salts thereof, as an absorption enhancer for IN delivery. Bile acids/salts are ionic amphiphilic compounds with a steroid skeleton. The physiological properties of bile acids/salts include lipid transport by solubilization and transport of drugs through hydrophobic barriers. Within the human body, bile acids/salts are amphipathic steroidal bio-surfactants that are derived from cholesterol in the liver. The synthesis of bile salts is the major route for elimination of cholesterol from the body. The concentration of bile salts in the human gall bladder is 0.5-2.5% and in portal vein blood is 0.1 mmol/L, i.e. approximately 50 mg/L, or 50 ppm.

Historically, bile acids/salts have not been successfully implemented for clinical use in human subjects due to various toxicity issues. In particular, bile acids/salts have limited clinical use because of the irreversible damage to the mucosa and ciliotoxicity. In addition prior to the compositions disclosed herein, bile acids/salts were understood to cause nasal irritation when used above a certain concentration, such as a concentration above 0.3% (3 mg/mL). Sodium taurocholate (STC) is an example of a bile salt. Sodium taurocholate (STC) is an example of such a bile salt. To date, the U.S. Food and Drug Administration (FDA) database does not list STC as an inactive ingredient for any approved drugs or drug formulations. Therefore, minimizing or reducing these bile salt's toxicity effects to the nasal cavity is a major technical challenge. Surprisingly, the IN compositions disclosed herein are well-tolerated by patients.

Embodiments of the present disclosure solve one or more of these pharmacological challenges and tolerance (or others) by introducing IN pharmaceutical formulations including an API, or a pharmaceutically acceptable salt thereof, and a bile acid, or a salt thereof, as the absorption enhancer. Also disclosed are methods of providing a rapid delivery of an API to a patient by IN delivery using the disclosed API formulations for various treatments, or indications.

Disclosed herein are formulations (e.g., pharmaceutical formulations) configured for intranasal delivery. In several embodiment, the formulation comprises an active ingredient, or a pharmaceutically acceptable salt thereof, and a bile acid (e.g., a bile acid or a salt thereof) as an absorption enhancer. In several embodiments, the absorption enhancer increases the absorption of the active pharmaceutical ingredient (API) within the nasal passages. In several embodiments, the bile acid, or the salt thereof, serves as the absorption enhancer for enhancing the absorption of an API into the bloodstream by intranasal delivery, as described herein. In several embodiments, the bile acid, or the salt thereof, is configured to enhance the absorption of an API into the bloodstream by intranasal delivery, as described herein. In several embodiments, the formulation comprises one or more carriers (e.g., pharmaceutically acceptable carriers) and/or excipients (e.g., pharmaceutically acceptable excipients) as disclosed elsewhere herein. In several embodiments, the pharmaceutical formulations for intranasal (IN) delivery are configured for use in a human subject. In several embodiments of the IN pharmaceutical formulations, the formulations include water (and/or are aqueous).

### Bile Acid/Salt Enhances Absorption of Active Ingredients by IN Delivery

The present disclosure introduces the safe use of a bile acid, or a salt thereof, as an absorption enhancer to enhance the absorption of an API into a human subject's bloodstream in IN delivery. Also disclosed are pharmaceutical formulations having an API and a bile acid, or a salt thereof, as an absorption enhancer to enhance the absorption of an API into a human subject's bloodstream in IN delivery. Bile acids/salts can enhance the IN absorption of an API into a human subject's bloodstream via the nasal mucosa. The nasal mucosa has two layers: (1) the outer epithelial layer, which is predominately lipophilic, and (2) the inner sublayer, known as the lamina propria, which comprises blood vessels for access to the bloodstream of the human subject. Without being bound to any theory, in several embodiments, the bile acid, or the salt thereof, enhances the IN absorption of the API by enabling access to the blood vessels in the lamina propria of the nasal mucosa. After the API is absorbed into the bloodstream, the API can be distributed throughout the human body via the circulatory system.

Bile acids are ionic amphiphilic compounds with a steroid skeleton. As demonstrated elsewhere herein, it has been found that bile acids have number of physiologically beneficial properties. In several embodiments, bile acids (or salts thereof) achieve lipid transport by solubilization of insoluble drug molecules. In several embodiments, the bile acid is configured to transport of polar drugs through hydrophobic barriers. In several embodiments, the bile acid inhibits enzyme activity. In several embodiments, without being bound to a theory, the bile acid aids in opening tight junctions between epithelial cells. In several embodiments, within the human body, bile acids are amphipathic and act as steroidal bio-surfactants. Bile acids are often derived from cholesterol in the liver. For example, the synthesis of bile salts is the major route for elimination of cholesterol from the body.

While the present disclosure is not limited by any particular mechanism or theory, it is believed that based on the above-described properties, bile acids/salts can enhance absorption of the API into the blood stream by forming micelles and/or reverse micelles to enable transcellular passage of the API through the predominantly lipophilic upper epithelial layer of the nasal mucosa and into the blood vessels located in the lamina propria sublayer. In several embodiments, one or more goals of the present disclosure (or others) are accomplished using a micelle or reverse micelle forming agent to enhance IN delivery (e.g., those including bile acids and/or bile acid salts). In several embodiments, the micelle or reverse micelle forming agent is used as an enhancing agent, as disclosed elsewhere herein. In several embodiments, the micelle or reverse micelle forming agent is a bile acid or bile acid salt. Without being bound to any particular theory, it is also believed that, in some embodiments, bile acids/salts inhibit the tight junctions between the epithelial cells to enable paracellular passage of the API through the predominantly lipophilic upper epithelial layer of the nasal mucosa and into the blood vessels located in the lamina propria sublayer. In this regard, bile acids/salts can disrupt the hemidesmosomes or by bind to calcium in the tight junctions. In several embodiments, one or more goals of the present disclosure (or others) are accomplished using a hemidesmosome disrupting agent to enhance IN delivery. In several embodiments, the hemidesmosome disrupting agent is used as an enhancing agent, as disclosed elsewhere herein. In several embodiments, the hemidesmosome disrupting agent is a bile acid or bile acid salt.

Additionally, in several embodiments, bile acids/salts can and/or are configured to enhance IN absorption of APIs by inhibiting, degrading, or reducing enzymes, such as mucosal membrane peptidases, in the predominantly lipophilic upper epithelial layer of the nasal mucosa. Without being bound to a theory, it is believed that bile acids/salts may enhance absorption of APIs by reducing the viscosity or elasticity of the predominantly lipophilic upper epithelial layer of the nasal mucosa. Therefore, bile acids/salts can enhance IN absorptions of APIs through these aforementioned means or a combination thereof. In several embodiments, the enhancing agent is an enzyme inhibiting agent. In several embodiments, the enhancing agent changes the viscosity and/or elasticity of the epithelial layer of the nasal mucosa. In several embodiments, the bile acid (or salt thereof) is used as an agent to inhibit enzymatic degradation of the API (e.g., epinephrine) and/or to change the viscosity and/or elasticity of the epithelial layer of the nasal mucosa (e.g., to enhance delivery of epinephrine).

Bile salts can be formed when the conjugated bile acid complexes with sodium or other appropriate cations. As disclosed elsewhere herein, other suitable elements (e.g., ions of elements and/or cations that form salts), such as potassium, may also be used to complex with the conjugate bile acid to form bile salts. Bile acids/salts can be conjugated with an amino acid, such as glycine or taurine, to form conjugated bile acids/salts. Bile acids/salts are ionic amphiphilic compounds with a steroid skeleton.

The structure below is a common chemical structure of a bile acid. As shown, this common structure of a bile acid consists of four rings, three six carbon rings (A, B and C), and one five carbon ring (D). The B ring may or may not be a double bond. The structure below is a non-limiting representative chemical structure of an embodiment of a bile acid:

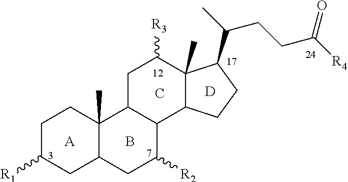

Wherein the numbering conforms to the steroid numbering system:

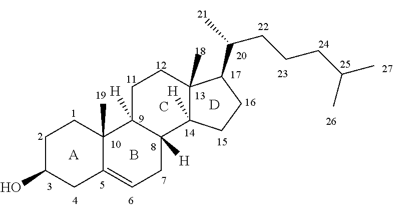

A number of APIs (e.g., epinephrine), by themselves, have low membrane permeability because they are hydrophilic and the upper epithelial layer of the nasal mucosa is predominantly lipophilic. For instance, when aqueous epinephrine is delivered by the IN route, the absorption is very low. The bioavailability (BA), based on the area under curve (AUC) in the plasma concentration of epinephrine, is only approximately 5% relative to that for the same dose of epinephrine delivered by the intramuscular (IM) route. Due to the difference between IM and IN route of administration, in order for epinephrine to reach a comparable absorption to that achieved by IM route of administration, an appropriate excipient is needed to enhance the absorption for IN delivery. STC and several other bile salt derivatives are identified to be excipients capable of enhancing delivery (e.g., enhancing agents).

However, historically, bile acids and its salts have not been successfully implemented for clinical use in human subjects due to various toxicity issues. In particular, bile acids/salts have limited clinical use because of the irreversible damage to the mucosa and ciliotoxicity. It has been reported that bile salts caused nasal irritation at equal to or above a concentration 0.3% (3 mg/mL). The side effects or toxic properties of hydrophobic bile acids are mostly produced when present in supraphysiological concentrations.

Thus, bile salts are currently only targeted for use in non-nasal delivery routes. For example, suppositories comprising a suppository base, a calcitonin and taurocholic acid or a pharmaceutically acceptable salt have been disclosed where the taurocholic acid servers as an enhancer. These formulations aimed at using a base containing calcitonin and taurocholic acid or its derivatives to improve bioavailability of suppository pharmaceutical compositions. Unlike those delivery routes, the current disclosure uses bile salts or its derivatives as for improving bioavailability of pharmacologically active components for nasal and pulmonary delivery. In several embodiments disclosed herein, the disclosed API formulations having STC as the bile salt is able to increase the bioavailability of the API by IN about 1.5-20 times compared to IN alone with no STC.

Bile acids/salts can be categorized into three main groups based on their conjugation with amino acids and their degree of hydroxylation. These three main groups are: (1) trihydroxy conjugates, (2) dihydroxy conjugates, and (3) unconjugated forms. In several embodiments, the bile acid and/or salt thereof of the IN pharmaceutical composition comprises a trihydroxy conjugate, a dihydroxy conjugate, an unconjugated form, or combinations of any of the foregoing. In several embodiments, combinations of bile acids and/or salts thereof may be used in the IN pharmaceutical formulation. In several embodiments, a plurality of different bile acids and/or salts thereof (e.g., 2, 3, 4, or more) may be used in the IN pharmaceutical formulation.

In several embodiments, the enhancing agent (e.g., absorption enhancer) in the IN pharmaceutical formulation is a trihydroxy conjugate (or a salt thereof). Exemplary embodiments of trihydroxy conjugates of bile acids that may be used in the intranasal formulations disclosed herein include, but are not limited to, glycocholate (GC), taurocholate (TC), glycohyocholate (GHC), taurohyocholate (THC), tauro-α-muricholate (T-α-MC), tauro-β-muricholate (T-β-MC), or a combination thereof. In several embodiments, the bile acid is taurocholic acid.

Exemplary embodiments of trihydroxy conjugates of bile salts that may be used in the intranasal formulations disclosed herein include, but are not limited to, sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), or a combination thereof. Other suitable forms of salts are possible, such as substituting sodium with potassium (e.g. potassium glycocholate). Other suitable forms of salts that may be used in the intranasal formulations disclosed herein are possible, such as substituting sodium with potassium (e.g. potassium glycocholate).

In several embodiments, the enhancing agent (e.g., absorption enhancer) in the IN pharmaceutical formulation is a dihydroxy conjugate (or a salt thereof). Exemplary embodiments of dihydroxy conjugates of bile acids that may be used in the intranasal formulations disclosed herein include tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination of any of the foregoing.

Exemplary embodiments of dihydroxy conjugates of bile salts that may be used in the intranasal formulations disclosed herein include sodium tauroursodeoxycholate (STUDC), sodium taurohyodeoxycholate (STHDC), sodium glycohyodeoxycholate (SGHDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination of any of the foregoing. Other suitable forms of salts that may be used in the intranasal formulations disclosed herein are possible, such as substituting sodium with potassium (e.g. potassium tauroursodeoxycholate).

In several embodiments, the enhancing agent (e.g., absorption enhancer) in the IN pharmaceutical formulation is a unconjugated bile acid (or a salt thereof). Exemplary embodiments of unconjugated forms of bile acids that may be used in the intranasal formulations disclosed herein include cholate, deoxycholate (DC), chenodeoxycholate (CDC), or a combination of any of the foregoing.

Exemplary embodiments of unconjugated forms of bile salts that may be used in the intranasal formulations disclosed herein include sodium cholate (SC), sodium deoxycholate (SDC), sodium chenodeoxycholate (SCDC), or a combination of any of the foregoing. Other suitable forms of salts that may be used in the intranasal formulations disclosed herein are possible, such as substituting sodium with potassium (e.g. potassium cholate). The bile salts of the present disclosure are not limited to those described above, and may include any other suitable bile salts.

In several embodiments, the bile acids/salts are configured to aggregate and/or to form micelles in concentrations above a critical micelle concentration (CMC). In several embodiments, by forming micelles, bile acids/salts can facilitate transcellular passage and enhance absorption through the nasal mucosa. In several embodiments, the bile acid and/or bile salt is provided at a concentration above its CMC. CMC values of certain exemplary bile salts are: sodium taurocholate (STC): CMC is ˜8 mM, sodium cholate (SC): CMC is ˜4 mM, sodium lithocholate (SLC): CMC is ˜1 mM, sodium glycocholate (SGC): CMC is ˜2⁻⁵ mM, sodium taurochenodeoxycholate (STCDC): CMC is ˜2.5-3 mM. In several embodiments, the bile acid of the has a CMC of equal to or at least about: 1 mM, 2 mM, 3 mM, 4 mM, 6 mM, 8 mM, 10 mM, or ranges including and/or spanning the aforementioned values.

In several embodiments of the IN pharmaceutical formulation, the bile acid, or the pharmaceutically acceptable salt thereof, is present at a concentration of equal to or less than about: 1 mg/mL, 3 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 20 mg/mL, or ranges including and/or spanning the aforementioned values. For example, in several embodiments, the IN pharmaceutical formulation comprises a bile acid or a salt thereof at a concentration ranging from 5.0 mg/mL to 15 mg/mL, 6 mg/mL to 14 mg/mL, 8 mg/mL to 12 mg/mL, 3 mg/mL to 20 mg/mL, etc.

In several embodiments, the IN pharmaceutical formulation comprises the bile acid, or the pharmaceutically acceptable salt thereof, at a molarity ratio to the API (e.g., bile acid or salt thereof: API) of equal to or less than about: 20:1, 15:1, 10:1, 7.5:1, 5:1, 4:1, 3:1, 2:1, 1:1, 1:2: 1:3, 1:4, 1:5, 1:10, or ranges including and/or spanning the aforementioned ratios. For example, in several embodiments, the IN pharmaceutical formulation comprises bile acid or a salt thereof at a molarity ratio to the API ranging from about: 20:1 to 1:10, 20:1 to 1:1, 5:1 to 1:5, 5:1 to 1:2, etc.

In several embodiments, the IN pharmaceutical formulation comprises the bile acid, or the pharmaceutically acceptable salt thereof, at a molarity of equal to or less than about: 0.007 M, 0.007 M, 0.009 M, 0.010 M, 0.011 M, 0.012 M, 0.013 M, 0.014 M, 0.015 M, 0.016 M, 0.019 M, 0.020 M, 0.022 M, 0.025 M, or ranges including and/or spanning the aforementioned values. For example, in several embodiments, the IN pharmaceutical formulation comprises bile acid or a salt thereof at a molarity ranging from 0.007 M to 0.022 M, 0.012 M to 0.020 M, 0.016 M to 0.025 M, 0.014 M to 0.019 M, etc.

In several embodiments of the IN pharmaceutical formulations, the bile acid, or the salt thereof, is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN pharmaceutical formulations, the bile acid, or the salt thereof, is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values.

In several embodiments of the IN pharmaceutical formulations, a bile acid, or the salt thereof, is present at a concentration of 5.0 mg/mL to 13.0 mg/mL. In several embodiments of the IN pharmaceutical formulations, the bile acid, or the salt thereof, is present at a concentration of equal to or less than about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, or ranges including and/or spanning the aforementioned values.

In several embodiments, a dose of the pharmaceutical formulation comprises a bile acid, or a pharmaceutically acceptable salt thereof, in an amount ranging from 0.1 mg to 1.8 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.70 mg to 0.90 mg, 0.70 mg to 0.80 mg, 1.0 mg to 1.4 mg, 0.9 mg to 1.3 mg, 1.0 mg to 1.4 mg, or 0.9 mg to 1.8 mg. In several embodiments, a dose of the pharmaceutical formulation comprises a bile acid, or a pharmaceutically acceptable salt thereof, in an amount of equal to or at least about: 0.10 mg, 0.15 mg, 0.20 mg, 0.25 mg, 0.30 mg, 0.35 mg, 0.40 mg, 0.45 mg, 0.50 mg, 0.55 mg, 0.60 mg, 0.65 mg, 0.70 mg, 0.75 mg, 0.80 mg, 0.85 mg, 0.90 mg, 0.95 mg, 1.00 mg, or ranges including and/or spanning the aforementioned values. In several embodiments, a dose of the pharmaceutical formulation comprises a bile acid, or a pharmaceutically acceptable salt thereof, in an amount of equal to or less than about: 0.80 mg, 0.85 mg, 0.90 mg, 0.95 mg, 1.00 mg, 1.05 mg, 1.10 mg, 1.15 mg, 1.20 mg, 1.25 mg, 1.30 mg, 1.35 mg, 1.40 mg, 1.45 mg, 1.50 mg, or ranges including and/or spanning the aforementioned values.

In several embodiments, a dose of the pharmaceutical formulation comprises a bile acid, or a pharmaceutically acceptable salt thereof, in an amount ranging from 0.5 mg to 1.3 mg. In several embodiments, a dose of the pharmaceutical formulation comprises a bile acid, or a pharmaceutically acceptable salt thereof, in an amount equal to or at least about: 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values. In several embodiments of IN pharmaceutical formulations, the bile acid, or the salt thereof, is present in a dose amount of equal to or less than about: 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values.

In several embodiments, the IN pharmaceutical formulation comprises API, including pharmaceutically acceptable salts thereof, at a concentration of 1.0 mg/mL to 25.0 mg/mL, an absorption enhancer comprising a bile acid, or a salt thereof, at a concentration of 5.0 mg/mL to 13.0 mg/mL, the pharmaceutical formulation has a pH of 2.2 to 5.0, and the pharmaceutical formulation is configured for IN delivery.

In several embodiments, the IN pharmaceutical formulation comprises API, including pharmaceutically acceptable salts thereof, present in a dose amount of 0.1 mg to 2.5 mg, an absorption enhancer comprising a bile acid, or a salt thereof, present in a dose amount of 0.5 mg to 1.3 mg, the pharmaceutical formulation has a pH of 2.2 to 5.0, and the pharmaceutical formulation is configured for IN delivery.

### Sodium Taurocholate (STC)

As an exemplary embodiment of a trihydroxy conjugate bile salt, sodium taurocholate (STC) is a trihydroxy conjugate bile salt that has the molecular formula C₂₆H₄₄NNaO₇S and a molecular weight (M.W.) of 537.7 g/mol. In several embodiments, the bile acid salt of the IN pharmaceutical formulation is STC. The chemical structure of STC is shown below:

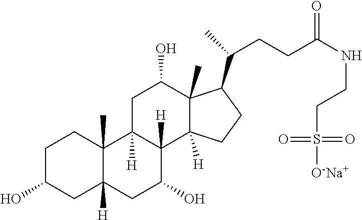

Other common examples of bile salts may include GDC: Glycodeoxychoate; SC: sodium cholate; SLC: sodium lithocholate; SGC: sodium glycocholate; STCDC, sodium taurochenodeoxycholate, and others as disclosed elsewhere herein, etc. STC is an ionic amphiphilic compound with a steroid skeleton. It belongs to the family of endogenous bile salts, important for multiple physiological functions including lipid transport of nutrients and drugs across hydrophobic barriers through the process of solubilization. As shown by the chemical structure of STC, STC has a hydrophobic portion that includes the steroid portion, and a hydrophilic portion. STC has a critical micelle concentration (CMC) of about 4 mg/mL (or approximately 8 mM). In several embodiments, STC is present in a concentration at or above its CMC.

One embodiment introduces a set of formulations of epinephrine IN delivery containing various concentrations of STC that demonstrates enhancement effects in human. These formulations are prepared for clinical use. Several nonlimiting formulations including STC are provided in the Examples. Another Example provided below discloses a randomized, active-controlled, evaluator-blinded, crossover study in healthy volunteers (male and female between 18 and 50 years old) that examines the enhancement of bioavailability of epinephrine using STC. Further, this example surprisingly shows the safety and efficacy of an exemplary API, epinephrine, using STC as an enhancer. This composition may be indicated for the emergency treatment of (and/or may be used in methods of treating) allergic reactions (Type I) including anaphylaxis to stinging insects (e.g., order Hymenoptera, which include bees, wasps, hornets, yellow jackets and fire ants) and biting insects (e.g., triatoma, mosquitoes), allergen immunotherapy, foods, drugs, diagnostic testing substances (e.g., radiocontrast media) and other allergens, as well as idiopathic anaphylaxis or exercise-induced anaphylaxis.

In the human PK study provided in the Examples, plasma concentration of the subjects treated with IN epinephrine with STC are tested and analyzed. Data for PK parameters, tₘₐₓ, Cₘₐₓ, and AUC_{0-t*}, based on the geometric mean and standard deviations are provided. The analysis results demonstrated that IN delivery of epinephrine with STC could reach a comparable higher Cₘₐₓ and AUC, compared to IM delivery of epinephrine. Furthermore, increasing amounts of STC may cause a quicker tₘₐₓ. Further again, in the same example, the PD data based on vital signs (heart rate, respiratory rate, systolic blood pressure, and diastolic blood pressure) at each PK time points are recorded and analyzed. The results demonstrated that the PD profiles for all treatments, regardless of epinephrine IM or IN epinephrine with various concentrations of STC, are comparable and demonstrate excellent safety features. The vital sign profiles for IN treatments with or without STC do not show significant differences.

Also in the Examples, a human safety study evaluating local irritations and tolerability, as well as adverse drug events (ADEs) are disclosed. Local irritation was assessed by Nasal and Oropharyngeal Mucosa Examination (NOME); Subjects Self-Reported Nasal Symptoms (SRNS); and University of Pennsylvania Smell Identification Test (UPSIT). All these assessment methods are international standards in the medical filed and required by the US FDA for drug approval. The assessments are conducted by third party ENT professionals. The safety study demonstrated that the impact of IN epinephrine with STC on the cardiovascular system and respiratory system are similar to that caused by the reference product (epinephrine by IM) based on PD and ADE profiles. Epinephrine/STC causes local irritations based on NOME, SRNS and ADE data with the following profile: (i) it causes a certain rate of mild to moderate local irritations (nasal oedema, nasal discomfort); (ii) but the probability of severe local irritation is low; and (iii) the reported local irritation is recoverable. The irritations recovered to baseline in about 2 weeks.

In several embodiments, as disclosed elsewhere herein, the bile salt is an STC. In several embodiments, the STC is an STC hydrate. In several embodiments, the STC or STC hydrate may be present in any amount or concentration disclosed elsewhere herein (e.g., at any amount or concentration provided for a bile acid salt or pharmaceutically acceptable bile acid salt). In several embodiments of the IN pharmaceutical formulations, the STC is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN pharmaceutical formulations, the STC is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values. In several embodiments of the IN pharmaceutical formulations, the STC is present at a concentration of equal to or less than about: 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, 16 mg/mL, 17 mg/mL, 18 mg/mL, 20 mg/mL, or ranges including and/or spanning the aforementioned values.

In several embodiments, as disclosed elsewhere herein, the bile salt is an STC, such as an STC hydrate. In several embodiments of the IN pharmaceutical formulations, the STC is present at a concentration of 5.0 mg/mL to 12.0 mg/mL. In several embodiments of the IN pharmaceutical formulations, the STC is present at a concentration of equal to or at least about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values. In several embodiments of the IN pharmaceutical formulations, the STC is present at a concentration of equal to or less than about: 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values.

In several embodiments, a dose of the pharmaceutical formulation comprises STC in an amount ranging from 0.1 mg to 1.5 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.7 mg to 0.9 mg, or 0.7 mg to 0.8 mg. In several embodiments, a dose of the pharmaceutical formulation comprises STC in an amount equal to or at least about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values. In several embodiments, a dose of the pharmaceutical formulation comprises STC in an amount equal to or less than about: 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values.

In several embodiments, a dose of the pharmaceutical formulation comprises STC in an amount ranging from 0.5 mg to 1.2 mg. In several embodiments, a dose of the pharmaceutical formulation comprises STC in an amount of equal to or at least about: 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, or ranges including and/or spanning the aforementioned values.

As disclosed elsewhere herein, in several embodiments, the IN pharmaceutical formulation comprises an API at a concentration of 1.0 mg/mL to 25.0 mg/mL, and an absorption enhancer comprising a bile salt present at a concentration of 5.0 mg/mL to 13.0 mg/mL, wherein the bile salt is STC. In several embodiments, the pharmaceutical formulation has a pH of 2.2 to 5.0. In several embodiments, the pharmaceutical formulation is configured for IN delivery.

In several embodiments, the IN pharmaceutical formulation comprises API present in a dose amount of 0.1 mg to 2.5 mg, an absorption enhancer comprising a bile salt present in a dose amount of 0.5 mg to 1.3 mg, wherein the bile salt is STC. In several embodiments, the pharmaceutical formulation has a pH of 2.2 to 5.0. In several embodiments, the pharmaceutical formulation is for IN delivery.

### Sodium Taurochenodeoxycholate (STCDC).

As another exemplary embodiment of a dihydroxy conjugate bile salt, sodium taurochenodeoxycholate (STCDC) is a dihydroxy conjugate bile salt that has the molecular formula C₂₆H₄₄NNaO₆S and a molecular weight (M.W.) of 521.7 g/mol and a CMC that is approximately 1.0-2.0 mg/mL (or 2.5-3.0 mM). In several embodiments, the bile acid salt of the IN pharmaceutical formulation is STCDC. The chemical structure of STCDC is shown below:

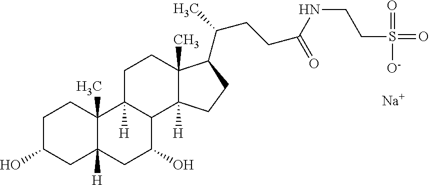

In several embodiments, the bile salt is an STCDC. In several embodiments, STCDC is present in a concentration at or above its CMC. In several embodiments of the IN pharmaceutical formulations, the STCDC is present at a concentration of 1.0 mg/mL to 15.0 mg/mL, or any concentration range subsumed therein, including but not limited to, 1.0 mg/mL to 12.5 mg/mL, 1.0 mg/mL to 10 mg/mL, 5.0 mg/mL to 11.0 mg/mL, 6.0 mg/mL to 13.0 mg/mL, 7.0 mg/mL to 12.0 mg/mL, 7.0 mg/mL to 9.0 mg/mL, 7.5 mg/mL to 9.5 mg/mL, 7.5 mg/mL to 8.5 mg/mL, 7.0 mg/mL to 9.0 mg/mL, or 7.0 mg/mL to 8.0 mg/mL. In several embodiments of the IN pharmaceutical formulations, the STCDC is present at a concentration of equal to or at least about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, or ranges including and/or spanning the aforementioned values. In several embodiments of the IN pharmaceutical formulations, the STCDC is present at a concentration of equal to or less than about: 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, 16 mg/mL, 17 mg/mL, 18 mg/mL, 20 mg/mL, or ranges including and/or spanning the aforementioned values.

In several embodiments of the IN pharmaceutical formulations, the STCDC is present at a concentration of 5.0 mg/mL to 12.0 mg/mL. In several embodiments of the IN pharmaceutical formulations, the STCDC is present at a concentration of equal to or at least about: 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values. In several embodiments of the IN pharmaceutical formulations, the STCDC is present at a concentration of equal to or less than about: 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, or 12.0 mg/mL, or ranges including and/or spanning the aforementioned values.

In several embodiments, a dose of the pharmaceutical formulation comprises STCDC in an amount ranging from 0.1 mg to 1.5 mg, or any amount range subsumed therein, including but not limited to, 0.6 mg to 1.3 mg, 0.5 mg to 1.1 mg, 0.7 mg to 1.2 mg, 0.7 mg to 0.9 mg, 0.75 mg to 0.95 mg, 0.75 mg to 0.85 mg, 0.7 mg to 0.9 mg, or 0.7 mg to 0.8 mg. In several embodiments, a dose of the pharmaceutical formulation comprises STCDC in an amount equal to or at least about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values. In several embodiments, a dose of the pharmaceutical formulation comprises STCDC in an amount equal to or less than about: 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, about 1.5 mg, or ranges including and/or spanning the aforementioned values.

In several embodiments, a dose of the pharmaceutical formulation comprises STCDC in an amount ranging from 0.5 mg to 1.2 mg. In several embodiments, a dose of the pharmaceutical formulation comprises STCDC in an amount of equal to or at least about: 0.50 mg, 0.55 mg, 0.60 mg, 0.65 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, or ranges including and/or spanning the aforementioned values.

As disclosed elsewhere herein, in several embodiments, the IN pharmaceutical formulation comprises API at a concentration of 1.0 mg/mL to 25.0 mg/mL, an absorption enhancer comprising a bile salt present at a concentration of 5.0 mg/mL to 13.0 mg/mL, wherein the bile salt is STCDC. In several embodiments, the pharmaceutical formulation has a pH of 2.2 to 5.0. In several embodiments, the pharmaceutical formulation is for IN delivery.

In several embodiments, the IN pharmaceutical formulation comprises API (including pharmaceutically acceptable salts thereof) present in a dose amount of 0.1 mg to 2.5 mg, and an absorption enhancer comprising a bile salt present in a dose amount of 0.5 mg to 1.3 mg. In several embodiments, the bile salt is STCDC. In several embodiments, the pharmaceutical formulation has a pH of 2.2 to 5.0. In several embodiments, the pharmaceutical formulation is for IN delivery.

### Bile Acid/Salt Enhancement Factor (EF)

The absorption enhancement effectiveness of the bile acid/salt can be quantified with reference to the following equation:

$$EF ( S ) = \frac{\overline{R} ( S )}{\overline{R} ( 0 )}$$

where EF(S) is the Bile Acid/Salt Enhancement Factor (“EF”)

R̅(S) is an average of the dose-normalized bioavailability (DN-RBA) for “X” number of PK parameters (e.g., 3 PK parameters: AUC_{0-30 min}, AUC_{0-∞}, and Cₘₐₓ) by the IN route at a given Bile Acid/Salt concentration S. Where simply referred to as the “Enhancement Factor” or “EF” without a PK subscript, what is meant is the enhancement factor calculated using AUC_{0-30 min}, AUC_{0-∞}, and Cₘₐₓ to calculate the dose normalized bioavailability. However, other AUC measures may be used to provide various other enhancement factors. In several embodiments, these other enhancement factors are reported herein using a subscript and listing the PK parameters used to calculate the enhancement factor (e.g., “EF_{PK1, PK2, PK3}”). For instance, an enhancement factor calculated using AUC_{0-30 min}, AUC_{0-180 min}, and Cₘₐₓ may be reported as EF_{AUC0-30/AUC0-180/Cmax} in Example 1. If an enhancement factor is reported as EF without a subscript, what is meant is the enhancement factor using AUC_{0-30 min}, AUC_{0-∞}, and Cₘₐₓ to calculate the dose-normalized bioavailability. Here, 3 parameters are used for purposes of illustration but X can be any number of parameters, including 1 parameter or more.

R̅(0) has the same definition at S=0, by the IN route.

Additionally, the dose-normalized relative bioavailability (DN-RBA) is defined as follows:

$${R}_{X} ( S ) = \frac{{X}_{IN} ( {d}_{IN} S ) \frac{1}{{d}_{IN}}}{{X}_{IM} ( {d}_{IM} ) \frac{1}{{d}_{IM}}}$$

where R_{X} is the DN-RBA for PK parameters X;

S is the concentration of Bile Acid/Salt (i.e. STC) used in the IN API formulations;

As noted above, X are AUC_{0-30 min}, AUC_{0-∞}, and Cₘₐₓ, note that AUC_{0-30 min}, AUC_{0-∞}, and Cₘₐₓ are used for illustrative purposes, other PK parameters can also be assessed. For example, in calculating an EF_{0-30/0-180/Cmax}, X are partial AUC, AUC_{0-30 min}, AUC_{0-180 min}, and Cₘₐₓ

d_{IM} and d_{IN} are doses delivered by IM and IN routes, respectively. As an example, the IM can be the 1 mg/mL API (e.g., epinephrine) by IM injection.

Example 1 will demonstrate the application of these principles to calculate EF_{AUC0-30/AUC0-180/Cmax} and Example 6 will demonstrate the application of these principles to calculate EF (e.g., EF_{AUC0-30/AUC0-∞/Cmax}). Example 1 will show that the IN formulations comprising the bile acid, or the salt thereof, as the absorption enhancer provided an Enhancement Factor_{0-30/0-180/Cmax} in a range of 1 to 23. In several embodiments, the EF is in a range of 1 to 23 based on an intranasal delivery (IN) v. intramuscular injection (IM) averaged pharmacokinetic (PK) results for AUC_{0-30 min}, AUC_{0-∞}, and Cₘₐₓ. In several embodiments, the EF_{0-30/0-180/Cmax} is in a range of 1 to 23 based on an intranasal delivery (IN) v. intramuscular injection (IM) averaged pharmacokinetic (PK) results for AUC_{0-30 min}, AUC_{0-180 min}, and Cₘₐₓ.

In several embodiments, the bile acid, or the salt thereof, provides an EF of at least 4, wherein the EF is determined based on R̅(S)/R̅(0), where R̅(S) is an average of a dose-normalized relative bioavailability (DN-RBA) of the pharmaceutical formulation relative to an IM injection having same API for two or more pharmacokinetic (PK) parameters, and R̅(0) is an average of DN-RBA of a pharmaceutical formulation without an absorption enhancer relative to an IM injection having same API for the two or more PK parameters. In several embodiments, the EF achieved using a bile acid or salt thereof is equal to or at least about: 1.5, 2, 3, 4, 6, 8, 9, 10, 11, 12, 13, 14, 15, or ranges including and/or spanning the aforementioned values.

In several embodiments, the bile acid, or the salt thereof, provides an enhancement factor in a range of 1 to 23 or any range subsumed therein, including, but not limited to, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, or 22 to 23. In several embodiments, the bile acid, or the salt thereof, provides an enhancement factor of at least 2, 3, 4, 5, 6, 7, 8, 9, 10, 11, 12, 13, 14, 15, 16, 17, 18, 19, 20, 21, 22, or 23. In several embodiments of the IN formulations, the absorption enhancer comprising the bile acid, or the salt thereof, provides an enhancement factor EF of more than 23.

In several embodiments, the enhancement factor is provided as EF_{PK1}, EF_{PK1, PK2}, or EF_{PK1, PK2, PK3}, where each of PK1, PK2, and PK3 are independently selected from a pharmacokinetic parameter. In several embodiments, PK1 is selected from Cₘₐₓ, tₘₐₓ, AUC_{0-t*}, AUC_{0-10 min}, AUC_{0-30 min}, AUC_{0-180 min}, AUC_{0-6 hr}, and AUC_{0-∞}. In several embodiments, where present, PK2 is selected from Cₘₐₓ, tₘₐₓ, AUC_{0-t*}, AUC_{0-10 min}, AUC_{0-30 min}, AUC_{0-180 min}, AUC_{0-6 hr}, and AUC_{0-∞}. In several embodiments, where present, PK3 is selected from is selected from Cₘₐₓ, tₘₐₓ, AUC_{0-t*}, AUC_{0-10 min}, AUC_{0-30 min}, AUC_{0-180 min}, AUC_{0-6 hr}, and AUC_{0-∞}. In several embodiments, where present in a given enhancement factor, each of PK1 and PK2 or PK1, PK2, and PK3 are different. In several embodiments, the EF_{PK1}, EF_{PK1, PK2}, or EF_{PK1, PK2, PK3} achieved using a bile acid or salt thereof is equal to or at least about: 4, 6, 8, 9, 10, 11, 12, 13, 14, 15, 20, or ranges including and/or spanning the aforementioned values. In several embodiments, the bile acid, or the salt thereof, provides an EF_{PK1}, EF_{PK1, PK2}, or EF_{PK1, PK2, PK3} in a range of 1 to 23 or any range subsumed therein, including, but not limited to, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, or 22 to 23. In several embodiments, the bile acid, or the salt thereof, provides an EF_{PK1}, EF_{PK1, PK2}, or EF_{PK1, PK2, PK3} of at least 2, 3, 4, 5, 6, 7, 8, 9, 10, 11, 12, 13, 14, 15, 16, 17, 18, 19, 20, 21, 22, or 23. In several embodiments of the IN formulations, the absorption enhancer comprising the bile acid, or the salt thereof, provides an EF_{PK1}, EF_{PK1, PK2}, or EF_{PK1, PK2, PK3} of more than 23.

In several embodiments, the PK parameter (e.g., PK1) is selected from the group consisting of Cₘₐₓ, tₘₐₓ, AUC_{0-t*}, AUC_{0-10 min}, AUC_{0-30 min}, AUC_{0-180 min}, AUC_{0-6 hr}, and AUC_{0-∞}. In several embodiments, the two or more PK parameters (e.g., PK1 and PK2; PK1, PK2, and PK3, etc.) are selected from the group consisting of Cₘₐₓ, tₘₐₓ, AUC_{0-t*}, AUC_{0-10 min}, AUC_{0-30 min}, AUC_{0-180 min}, AUC_{0-6 hr}, and AUC_{0-∞}. In several embodiments, the two or more PK parameters include two or more of Cₘₐₓ, AUC_{0-t*}, and AUC_{0-30 min}. In several embodiments, the two or more PK parameters include tₘₐₓ, AUC_{0-t*}, and AUC_{0-6 hr}. In several embodiments, the two or more PK parameters include Cₘₐₓ, AUC_{0-30 min}, and AUC_{0-6 hr}, and AUC_{0-∞}. In several embodiments, the two or more PK parameters include Cₘₐₓ, AUC_{0-t*}, and AUC_{0-∞}. In several embodiments, the two or more PK parameters include AUC_{0-30 min}, AUC_{0-180 min}, and Cₘₐₓ. In other embodiments, the two or more PK parameters include AUC_{0-10 min}, AUC_{0-15 min}, AUC_{0-30 min}, AUC_{0-45 min}, AUC_{0-60 min}, AUC_{0-75 min}, AUC_{0-90 min}, AUC_{0-100 min}, AUC_{0-125 min}, AUC_{0-150 min}, AUC_{0-180 min}, AUC_{0-infinity}, Cₘₐₓ, tₘₐₓ, other suitable PK parameters, or any combination thereof.

Bile Acids and/or Salts Thereof can Enhance Absorption of Small and Large Molecules

In the drug industry, because of their small size and typical physiochemical properties, small molecules can be effective enzyme inhibitors and allosteric modifiers and can target extracellular proteins or intracellular receptors in the cytosol, nuclei, and central nervous system. Despite their perceived limitations, and with a recent resurgence, small molecules remain the major component of an ever-expanding therapeutic toolbox. However, in recent years, proteins/biologics have become more and more popular. For example, biologic proteins are capable of performing highly specific and complex functions, which is impossible for small molecule drugs. The high specificity of proteins also may result in less drug toxicity through interference with normal body processes. Small molecule drugs and therapeutic proteins differ substantially in many of their class attributes. At the root of this are their different physiochemical properties, which affect not only the pharmacological aspects of the drug (e.g., mechanism of action, pharmacodynamics (PD), pharmacokinetics (PK), but also safety and efficacy, and even impact product manufacturing/quality considerations.

Surprisingly, the current invention demonstrates that bile salts can be used as an excipient added to improve the IN absorption of both small molecules, such as epinephrine and naloxone, and biologics, complex molecules, such as insulin aspart. Bile acids or salts can significantly enhance absorption in IN delivery of a wide range of APIs including small and large size APIs, as exemplified by nonclinical rat model described in the examples. These examples demonstrate the safe use of different bile salts for different APIs (naloxone and epinephrine) and different formulations with different bile salt concentrations. The nasal cavity irritations and tolerability of these STC containing formulations were examined based on both macroscopically and microscopically histopathological findings. The results demonstrated the findings are with minimal severity and all damages/findings can be repaired or reversed in one (1) week to the negative control group level.

It is important to emphasize that, in the disclosed formulations, the mucosa damage is reversible and can be repaired if the concentration of bile salt is not more than 15 mg/mL.

As disclosed elsewhere herein, a bile acid, or the salt thereof, can be utilized to enhance absorption of any API into a human subject's bloodstream in IN delivery. For example, the bile acid, or the salt thereof, of the present disclosure can be utilized to enhance absorption into the bloodstream in IN delivery of a thiazide, a protein, an immunosuppressive drug, an antidiarrhoeal, a reuptake inhibitor, an anesthetic, an antihistamine, a cannabinoid, a dietary supplement (e.g., a vitamin), a proton-pump inhibitor, an anti-hypertensive drug, an antiviral drug, a statin, an anxiolytic, a corticosteroid, an anticoagulant, an anti-inflammatory drug (e.g., a steroidal and/or non-steroidal anti-inflammatory drug), a diuretic, an anti-convulsant, an anti-psychotic drug, an antidepressant, a barbituate, a narcotic, a beta blocker, an antibiotic, an agonist drug, an angiotensin-converting enzyme (ACE) inhibitor, a depressant, a steroid (e.g., a corticosteroid and/or an anabolic steroid), a sedative, an analgesic, a benzodiazepine, an antagonist drug, an opioid, a stimulant, and/or an enzyme inhibitor.

As is demonstrated by the Examples described herein below, the bile acid, or the salt thereof, can enhance the absorption into the bloodstream in IN delivery of a wide range of APIs including small and large size APIs.

In several embodiments, as described elsewhere herein, the composition comprises the API as a neutral compound, a free acid, a free base, or as a pharmaceutically acceptable salt. In several embodiments, the pharmaceutically acceptable salt is as disclosed elsewhere herein. In several embodiments, the pharmaceutically acceptable salt is an acetate salt, a bitartrate salt, a carbonate salt, a citrate salt, a hydrochloride salt, a hydrocyanide salt, a hydrofluoride salt, a nitrate salt, a nitrite salt, a phosphate salt, a sulfate salt, or a combination of any one or more of the foregoing. In several embodiments, the present disclosure is not limited to these salt forms.

In several embodiments, the API or the pharmaceutically acceptable salt thereof is present at a concentration of equal to or less than about: 1 mg/mL, 2.5 mg/mL, 5 mg/mL, 7.5 mg/mL, 10 mg/mL, 11 mg/mL, 12 mg/mL, 13 mg/mL, 14 mg/mL, 15 mg/mL, 20 mg/mL, or ranges including and/or spanning the aforementioned values. For example, in several embodiments, the IN pharmaceutical formulation comprises API or the pharmaceutically acceptable salt thereof at a concentration ranging from 7.5 mg/mL to 15 mg/mL, 10 mg/mL to 14 mg/mL, 5 mg/mL to 15 mg/mL, 10 mg/mL to 20 mg/mL, etc.

In several embodiments, the API or the pharmaceutically acceptable salt thereof is present at a concentration of 1.0 mg/mL to 25.0 mg/mL, or any concentration range subsumed therein, including but not limited to, 5.0 mg/mL to 15.0 mg/mL, 5.0 mg/mL to 13.0 mg/mL, 7.5 mg/mL to 12.5 mg/mL, 8.0 mg/mL to 12.0 mg/mL, 9.0 mg/mL to 11.0 mg/mL, 9.5 mg/mL to 10.5 mg/mL, or 7.0 mg/mL to 9.0 mg/mL. In several embodiments, the API or the pharmaceutically acceptable salt thereof is present at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 10.5 mg/mL, 11.0 mg/mL, 11.5 mg/mL, 12.0 mg/mL, 12.5 mg/mL, 13.0 mg/mL, 13.5 mg/mL, 14.0 mg/mL, 14.5 mg/mL, 15.0 mg/mL, 15.5 mg/mL, 16.0 mg/mL, 16.5 mg/mL, 17.0 mg/mL, 17.5 mg/mL, 18.0 mg/mL, 18.5 mg/mL, 19.0 mg/mL, 19.5 mg/mL, 20.0 mg/mL, 20.5 mg/mL, 21.0 mg/mL, 21.5 mg/mL, 22.0 mg/mL, 22.5 mg/mL, 23.0 mg/mL, 23.5 mg/mL, 24.0 mg/mL, 24.5 mg/mL, 25.0 mg/mL, or ranges including and/or spanning the aforementioned values.

In several embodiments, the API or the pharmaceutically acceptable salt thereof is present at a concentration of 5.0 mg/mL to 13.0 mg/mL. In several embodiments, the API or the pharmaceutically acceptable salt thereof is present at a concentration of equal to or less than about: 5.0 mg/mL, 6.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, 11.5 mg/mL, 12.0 mg/mL, 12.5 mg/mL, 13.0 mg/mL, or ranges including and/or spanning the aforementioned values.

In several embodiments, the API or the pharmaceutically acceptable salt thereof is present within the formulation at a molarity of equal to or less than about: 0.005 M, 0.02 M, 0.04 M, 0.05 M, 0.06 M, 0.07 M, 0.08M, 0.1 M, 0.15 M, or ranges including and/or spanning the aforementioned values. For example, in several embodiments, the IN pharmaceutical formulation comprises API or the pharmaceutically acceptable salt thereof at a molarity ranging from 0.04 M to 0.07 M, 0.05 M to 0.07 M, 0.02 M to 0.1 M, 0.05 M to 0.07 M, etc.

In several embodiments, as disclosed elsewhere herein, the formulation is provided in a IN dosing device. In several embodiments, the dosing device delivers a dose of the composition to a patient (e.g., a patient in need of treatment). In several embodiments, a dose of the pharmaceutical formulation comprises API or the pharmaceutically acceptable salt thereof in an amount ranging from 0.1 mg to 5.0 mg, or any amount range subsumed therein, including but not limited to, 0.1 mg to 4.5 mg, 0.1 mg to 4.25 mg, 0.1 mg to 4.0 mg, 0.1 mg to 3.5 mg, 0.1 mg to 3.25 mg, 0.1 mg to 3.0 mg, 0.1 mg to 2.75 mg, 0.1 mg to 2.5 mg, 0.1 mg to 2.25 mg, 0.1 mg to 2.0 mg, 0.1 mg to 1.75 mg, 0.1 mg to 1.5 mg, 0.1 mg to 1.25 mg, 0.1 mg to 1.0 mg, 0.1 mg to 0.75 mg, 0.1 mg to 0.5 mg, 0.1 mg to 0.25 mg, 0.25 mg to 5.0 mg, 0.25 mg to 4.5 mg, 0.25 mg to 4.25 mg, 0.25 mg to 4.0 mg, 0.25 mg to 3.5 mg, 0.25 mg to 3.25 mg, 0.25 mg to 3.0 mg, 0.25 mg to 2.75 mg, 0.25 mg to 2.5 mg, 0.25 mg to 2.25 mg, 0.25 mg to 2.0 mg, 0.25 mg to 1.75 mg, 0.25 mg to 1.5 mg, 0.25 mg to 1.25 mg, 0.25 mg to 1.0 mg, 0.25 mg to 0.75 mg, 0.25 mg to 0.5 mg, 0.5 mg to 4.5 mg, 0.5 mg to 4.25 mg, 0.5 mg to 4.0 mg, 0.5 mg to 3.5 mg, 0.5 mg to 3.25 mg, 0.5 mg to 3.0 mg, 0.5 mg to 2.75 mg, 0.5 mg to 2.5 mg, 0.5 mg to 2.25 mg, 0.5 mg to 2.0 mg, 0.5 mg to 1.75 mg, 0.5 mg to 1.5 mg, 0.5 mg to 1.3 mg, 0.5 mg to 1.25 mg, 0.5 mg to 1.0 mg, 0.5 mg to 0.75 mg, 0.75 mg to 5.0 mg, 0.75 mg to 4.5 mg, 0.75 mg to 4.25 mg, 0.75 mg to 4.0 mg, 0.75 mg to 3.5 mg, 0.75 mg to 3.25 mg, 0.75 mg to 3.0 mg, 0.75 mg to 2.75 mg, 0.75 mg to 2.5 mg, 0.75 mg to 2.25 mg, 0.75 mg to 2.0 mg, 0.75 mg to 1.75 mg, 0.75 mg to 1.5 mg, 0.75 mg to 1.3 mg, 0.75 mg to 1.25 mg, 0.75 mg to 1.0 mg, 1.0 mg to 5.0 mg, 1.0 mg to 4.0 mg, 1.0 mg to 3.0 mg, 1.0 mg to 2.0 mg, 2.0 mg to 5.0 mg, 2.0 mg to 4.0 mg, 2.0 mg to 3.0 mg, 3.0 mg to 5.0 mg, 3.0 mg to 4.0 mg, 4.0 mg to 5.0 mg, or ranges including and/or spanning any of the aforementioned values.

In several embodiments, a dose of the pharmaceutical formulation comprises API or the pharmaceutically acceptable salt thereof in an amount equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, or ranges including and/or spanning the aforementioned values.

In several embodiments, a dose of the pharmaceutical formulation comprises API or the pharmaceutically acceptable salt thereof in an amount ranging from 0.1 mg to 2.5 mg, or any amount range subsumed therein, including but not limited to, 0.5 mg to 1.5 mg, 0.5 mg to 1.3 mg, 0.7 mg to 0.9 mg, 0.75 mg to 1.25 mg, 0.8 mg to 1.2 mg, 0.9 mg to 1.1 mg, 0.95 mg to 1.05 mg. In several embodiments, a dose of the pharmaceutical formulation comprises API or the pharmaceutically acceptable salt thereof in an amount equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, or 2.5 mg, or ranges including and/or spanning the aforementioned values.

In several embodiments, a dose of the pharmaceutical formulation comprises API or the pharmaceutically acceptable salt thereof in an amount ranging from 0.5 mg to 1.30 mg. In several embodiments, a dose of the pharmaceutical formulation comprises API or the pharmaceutically acceptable salt thereof in an amount equal to or less than about: 0.50 mg, 0.55 mg, 0.60 mg, 0.70 mg, 0.75 mg, 0.80 mg, 0.85 mg, 0.90 mg, 0.95 mg, 1.00 mg, 1.05 mg, 1.10 mg, 1.15 mg, 1.20 mg, 1.25 mg, 1.30 mg, or ranges including and/or spanning the aforementioned values.

In several embodiments of the pharmaceutical formulations, a single administration (e.g., a single spray) provides a full dose of the pharmaceutical formulation. In several embodiments, a dose can be provided in a plurality of administrations from dosing device. For example, multiple sprays (e.g., equal to or greater than 2, 3, 4, 5 sprays) in quick succession. In the context of IN delivery, “to be taken at one time” covers the discharge of the dose volume in: (1) a single spray, or (2) two or more sprays in a very short amount of time, usually less than one minute. Thus, the dose volume containing the dose amount of API or the pharmaceutically acceptable salt thereof can be discharged in one or more nasal sprays. In several embodiments, the dose volume is discharged in one spray of the nasal spray. In several embodiments, the dose volume is discharged in two or more sprays of the nasal spray (e.g., 2, 3, 4, 5, or more sprays). In several embodiments, the dose amount of the API or the pharmaceutically acceptable salt thereof is discharged in a single spray. In several embodiments, the dose amount of the API or the pharmaceutically acceptable salt thereof is discharged in two or more sprays (e.g., 2, 3, 4, or more sprays).

In several embodiments, the dose volume of the IN pharmaceutical formulation is from 0.01 mL to 0.30 mL. In several embodiments, the dose volume of the IN pharmaceutical formulation is from 0.05 mL to 0.15 mL. In several embodiments, the dose volume of the IN pharmaceutical formulation is about 0.10 mL. In several embodiments of the IN pharmaceutical formulation, the dose volume is about 0.10 mL, which can be discharged in a single nasal spray. In several embodiments, a dose volume of the IN pharmaceutical formulation equal to or less than about: 0.01 mL, 0.05 mL, 0.075 mL, 0.1 mL, 0.2 mL, 0.3 mL, or ranges including and/or spanning the aforementioned values.

### Small Molecule APIs

As disclosed elsewhere herein, in several embodiments, the API is a small molecule. A small molecule API is generally a chemically synthesized API (e.g., synthetic) that may be synthesized by one or more chemical reactions. In several embodiments, the small molecule API has a molecular weight of less than 1000 g/mol, such as in a range of 0 to 1000 g/mol or any range subsumed therein. In several embodiments, the small molecule API has a molecular weight (in g/mol) of less than or equal to about: 150, 200, 250, 300, 350, 400, 500, 600, 700, 800, 900, 1000, or ranges including and/or spanning the aforementioned values. As used herein, the units “g/mol” are used synonymously with Daltons (Da). Epinephrine (molecular weight of about 183 g/mol) and naloxone (molecule weight of about 327 g/mol) are examples of small molecule APIs.

Within small molecule APIs, there are a variety of APIs suitable for IN delivery and that can be utilized together with the bile acid, or the salt thereof, of the present disclosure to enhance absorption into the bloodstream in IN delivery. For example, in several embodiments, the small molecule API may include an alkaloid, a glycoside, a lipid, phenazine, a phenol, a polyketide, a terpene (e.g., a steroid), and/or a tetrapyrrole. In several embodiments, the small molecule API is an adrenergic agonist. In some embodiments, the adrenergic agonist includes, but is not limited to, epinephrine, norepinephrine, dopamine, isoprenaline, phenylephrine, dexmedetomidine, oxymetazoline, methyldopa, clonidine, dobutamine, salbutamol, albuterol, terbutaline, salmeterol, formoterol, and/or pirbuterol. In several embodiments, the small molecule API is an opioid antagonist. In several embodiments, the opioid antagonist includes, but is not limited to, naloxone, nalmefene, and/or naltrexone. In several embodiments, the API is not naloxone and/or epinephrine.

### Large Molecule APIs

In several embodiments, the pharmaceutical formulation comprises a large molecule API. In several embodiments, the large molecule API may have a molecular weight of 1,000 g/mol or more. For example, the large molecule API may have a molecular weight of 1,000 g/mol to 250,000 g/mol any range subsumed therein, including but not limited to, 1,000 g/mol to 200,000 g/mol, 1,000 g/mol to 100,000 g/mol, 1,000 g/mol to 75,000 g/mol, 1,000 g/mol to 50,000 g/mol, or 1,000 g/mol to 10,000 g/mol. In several embodiments, the large molecule API has a molecular weight (in g/mol) of greater or equal to about: 1,000, 2,000, 3,000, 5,000, 10,000, 20,000, 40,000, 80,000, 100,000, or ranges including and/or spanning the aforementioned values. In some embodiments, the large molecule API is a biologic. In some embodiments, the large molecule API is a protein API. Insulin aspart (molecular weight of about 5826 g/mol) is an example of a protein API or a large molecule API. Other examples of protein APIs include insulin glargine and recombinant human insulin.

With respect to the large molecule API, there are a variety of APIs suitable for IN delivery and that can be utilized together with the bile acid, or the salt thereof, of the present disclosure to enhance absorption into the bloodstream during IN delivery. For example, the large molecule API may include any suitable biologic. Examples of the large molecule API (or biologic) include proteins (e.g., recombinant proteins) and/or nucleic acids (e.g., recombinant nucleic acids). Examples of the proteins include antibodies (e.g., monoclonal antibodies), antitoxins (e.g., antivenin), hormones (e.g., insulin), cytokines (e.g., interleukins), enzymes, tumor necrosis factors, antigens, interferons, haematopoietic growth factors (e.g., erythropoietin), blood factors, and/or thrombolytic agents. In some embodiments, the insulin may include human insulin and/or recombinant insulin such as, for example, insulin aspart and/or insulin glargine.

IN API Pharmaceutical Formulations

In some embodiments, the disclosed pharmaceutical formulations comprise an active pharmaceutical ingredient (API), and an absorption enhancer comprising a bile acid, or a salt thereof, wherein the bile acid, or the salt thereof, enhances absorption of the API by IN delivery in a human subject. Also disclosed are methods of delivering an active pharmaceutical ingredient, the method comprising administering a pharmaceutical formulation to a human subject by intranasal (IN) delivery using a nasal spray, wherein the pharmaceutical formulation comprises a therapeutically effective amount of the active pharmaceutical ingredient and an absorption enhancer comprising a bile acid, or a salt thereof, and wherein the bile acid, or the salt thereof, enhances absorption of the API by IN delivery in the human subject.

Advantageously, in some embodiments of the pharmaceutical formulations or corresponding methods, the bile acid, or the salt thereof, is present at a concentration of at least 3.0 mg/mL per dose volume. In other embodiments, the bile acid, or the salt thereof, is present at a concentration of 3.0 mg/mL to 15.0 mg/mL per dose volume. In still other embodiments, the bile acid, or the salt thereof, is present at a concentration of 5.0 mg/mL to 13.0 mg/mL per dose volume. The dose volume contains the dose amount of the API to be taken at one time.

Advantageously, in some embodiments of the pharmaceutical formulations or corresponding methods, the bile acid, or the salt thereof, is present at a dose amount of at least 0.3 mg. In other embodiments, the bile acid, or the salt thereof, is present at a dose amount of 0.3 mg to 1.5 mg. In still other embodiments, the bile acid, or the salt thereof, is present at a dose amount of 0.5 mg to 1.3 mg.

In some embodiments of the pharmaceutical formulations or corresponding methods, the bile acid, or the salt thereof, comprises a trihydroxy conjugate. In other embodiments, the bile acid is a trihydroxy conjugate comprising glycocholate (GC), taurocholate (TC), glycohyocholate (GHC), taurohyocholate (THC), tauro-α-muricholate (T-α-MC), tauro-β-muricholate (T-β-MC), or a combination thereof. In still other embodiments, the bile salt is a trihydroxy conjugate comprising sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), or a combination thereof. Other suitable forms of salts are possible, such as substituting sodium with potassium (e.g. potassium glycocholate).

In some embodiments of the pharmaceutical formulations or corresponding methods, the bile acid, or the salt thereof, is a dihydroxy conjugate. In other embodiments, the bile salt is a dihydroxy conjugate comprising comprises sodium tauroursodeoxycholate (STUDC), sodium taurohyodeoxycholate (STHDC), sodium glycohyodeoxycholate (SGHDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination thereof. In still other embodiments, the bile acid is a dihydroxy conjugate comprising tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination thereof.

In some embodiments of the pharmaceutical formulations or corresponding methods, the bile acid, or the salt thereof, is an unconjugated form. In other embodiments, the bile acid is an unconjugated form comprising cholate, deoxycholate (DC), chenodeoxycholate (CDC), or a combination thereof. In still other embodiments, the bile salt is an unconjugated form comprising sodium cholate (SC), sodium deoxycholate (SDC), sodium chenodeoxycholate (SCDC), or a combination thereof.

In some embodiments of the pharmaceutical formulations or corresponding methods, the API is a small molecule having a molecular weight of less than 900 g/mol. In other embodiments, the API is a small molecule comprising an adrenergic agonist. In still other embodiments, the API is a small molecule comprising an adrenergic agonist, wherein the adrenergic agonist includes epinephrine, norepinephrine, dopamine, isoprenaline, phenylephrine, dexmedetomidine, oxymetazoline, methyldopa, clonidine, dobutamine, salbutamol, albuterol, terbutaline, salmeterol, formoterol, or pirbuterol. In other embodiments, the API is a small molecule comprising an opioid antagonist. In certain embodiments, the API is a small molecule comprising an opioid antagonist, the opioid antagonist includes naloxone, nalmefene, and/or naltrexone.

In some embodiments of the pharmaceutical formulations or corresponding methods, the API is a large molecule having a molecular weight of 900 g/mol or more. In other embodiments, the API is a large molecule comprising a protein, wherein the protein includes insulin, insulin aspart, or insulin glargine.

In certain embodiments of the pharmaceutical formulations or corresponding methods, the disclosed pharmaceutical formulations further include other excipients. By way of examples, excipients may include a tonicity agent, an antioxidant, a preservative, a buffer, a pH adjustor, a metal complexing agent, other known excipients, or a combination thereof.

### IN API Pharmaceutical Formulations May Include Pharmaceutically Acceptable Excipients

The disclosed IN API pharmaceutical formulations further include one or more pharmaceutically acceptable excipients.

### pH Adjusting or pH Stabilizing Agents

In several embodiments, the pH of the pharmaceutical formulation is acidic. In several embodiments, the pH of the pharmaceutical formulation is 2.2 to 7.0, or any pH range subsumed therein, including but not limited to, 3.0 to 4.5, 3.0 to 3.5, 3.5 to 4.0, 3.7 to 3.9, 3.75 to 3.85, 4.0 to 4.5, or 4.5 to 5.0. In several embodiments, the pH of the pharmaceutical formulation is equal to or less than about: 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, or ranges including and/or spanning the aforementioned values. For example, in several embodiments, the pH of the pharmaceutical formulation ranges from 3.2 to 4.5, 3.4 to 5.0, from 3.7 to 3.9, etc.

In several embodiments, the pH of the pharmaceutical formulation is basic. In several embodiments, the pH of the pharmaceutical formulation is 7.0 to 10.5, or any pH range subsumed therein, including but not limited to, 7.0 to 8.5, 7.0 to 9.5, 8.5 to 10.0, 7.1 to 10.5, 7.5 to 10.5, etc. In several embodiments, the pH of the pharmaceutical formulation is equal to or greater than about: 7.0, 7.5, 8.0, 8.5, 9.0, 9.5, 10.0, 10.5, or ranges including and/or spanning the aforementioned values. For example, in several embodiments, the pH of the pharmaceutical formulation ranges from 7.0 to 8.5, 7.0 to 9.5, 8.5 to 10.0, 7.5 to 10.5, etc.

In several embodiments, the pharmaceutical formulations further includes a buffer (e.g., a buffer system). In several embodiments, the buffer comprises one or more of citric acid, sodium citrate, sodium phosphate, or a combination thereof, but the present disclosure is not limited thereto. In several embodiments, the buffer system comprises an acid and its conjugate base. In several embodiments, the buffer system comprises a base and its conjugate acid. In several embodiments, the buffer can include a first buffer agent (e.g., an acid), such as citric acid, and a second buffer agent (e.g., a conjugate base), such as sodium citrate, thereby forming a buffer pair. In some embodiments, the acid (e.g., conjugate acid) is adipic acid, ammonium chloride, citric acid, acetic acid, formic acid, lactic acid, phosphoric acid, propionic acid, tartaric acid, combinations of the foregoing, or other acids. In several embodiments, the base (e.g., conjugate base) is acetate (e.g., sodium acetate, etc.), citrate (e.g., sodium citrate, etc.), bicarbonate (e.g., sodium bicarbonate, etc.), carbonate (e.g., sodium carbonate), lactate (e.g., sodium lactate, etc.), phosphate (e.g., sodium phosphate), combinations of the foregoing, or other bases. In several embodiments, the buffer is a phosphate buffer, an acetate buffer, or a citrate buffer. In several embodiments, the buffer is a citrate buffer. In several embodiments, the buffer is a MES hydrate or monohydrate buffer. In several embodiments, the buffer is a BIS TRIS buffer.

In several embodiments of the pharmaceutical formulations, the buffer includes an acid (e.g., conjugate acid). In several embodiments, the acid (e.g., conjugate acid) is present at a concentration of 1.0 mg/mL to 8.0 mg/mL, or any concentration range subsumed therein, including but not limited to, 2.0 mg/mL to 7.0 mg/mL, 1.5 mg/mL to 6.5 mg/mL, 2.0 mg/mL to 7.0 mg/mL, or 3.0 mg/mL to 5.0 mg/mL. In several embodiments of the pharmaceutical formulations, the acid (e.g., conjugate acid) is present at a concentration of equal to or less than about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, or ranges including and/or spanning the aforementioned values. In several embodiments of the pharmaceutical formulations, the buffer includes citric acid (or a citric acid source) present at a concentration of 1.0 mg/mL to 8.0 mg/mL, or any concentration range subsumed therein, including but not limited to, 2.0 mg/mL to 7.0 mg/mL, 1.5 mg/mL to 6.5 mg/mL, 2.0 mg/mL to 7.0 mg/mL, or 3.0 mg/mL to 5.0 mg/mL. In several embodiments of the pharmaceutical formulations, the citric acid (or citric acid source) is present at a concentration of about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, or ranges including and/or spanning the aforementioned values. In several embodiments of the pharmaceutical formulations, the citric acid source is citric acid monohydrate.

In several embodiments of the pharmaceutical formulations, the buffer includes a base (e.g., a conjugate base) present at a concentration of 1.0 mg/mL to 10.0 mg/mL, or any concentration range subsumed therein, including but not limited to, 5.0 mg/mL to 10.0 mg/mL, 2.0 mg/mL to 8.0 mg/mL, 4.0 mg/mL to 7.0 mg/mL, 7.0 mg/mL to 9.0 mg/mL, or any concentration range subsumed therein. In several embodiments of the pharmaceutical formulations, the base (e.g., a conjugate base) is present at a concentration of equal to or less than about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, or ranges including and/or spanning the aforementioned values. In several embodiments of the pharmaceutical formulations, the buffer includes sodium citrate (or a sodium citrate source) present at a concentration of 1.0 mg/mL to 10.0 mg/mL, or any concentration range subsumed therein, including but not limited to, 5.0 mg/mL to 10.0 mg/mL, 2.0 mg/mL to 8.0 mg/mL, 4.0 mg/mL to 7.0 mg/mL, 7.0 mg/mL to 9.0 mg/mL, or any concentration range subsumed therein. In several embodiments of the pharmaceutical formulations, the sodium citrate (or sodium citrate source) is present at a concentration about: 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, or ranges including and/or spanning the aforementioned values. In several embodiments of the pharmaceutical formulations, the sodium citrate source is a sodium citrate dihydrate. In several embodiments of the pharmaceutical formulations, the buffer includes citric acid and sodium citrate.

In several embodiments of the pharmaceutical formulations, the buffer includes citric acid in a concentration range of 3.0 mg/mL to 5.0 mg/mL and sodium citrate in a concentration range of 6.0 mg/mL to 10.0 mL. In several embodiments of the pharmaceutical formulations, the buffer includes citric acid having a concentration of about 4.0 mg/mL and sodium citrate having a concentration of about 8.0 mg/mL.

In several embodiments, the pharmaceutical formulation comprises a buffer (e.g., the acid and conjugate base; the conjugate acid and base pair; etc.), at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values. For example, in several embodiments, the pharmaceutical formulation comprises the buffer at a molarity ranging from 0.01 M to 0.1 M, 0.02 M to 0.08 M, 0.06 M to 0.1 M, 0.05 M to 0.2 M, etc. In several embodiments, the pharmaceutical formulation comprises the buffer is a citrate buffer at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values. In several embodiments, the pharmaceutical formulation comprises the buffer is an acetate buffer at a molarity of equal to or less than about: 0.01 M, 0.02 M, 0.03 M, 0.04 M, 0.05 M, 0.06 M, 0.07 M, 0.08 M, 0.09 M, 0.1 M, or ranges including and/or spanning the aforementioned values.

### Preservatives

In several embodiments, the pharmaceutical formulations further include a preservative. In several embodiments, the preservative is selected from the group consisting of chlorobutanol, parabens (e.g., methyl paraben), phenyl ethyl alcohol, benzalkonium chloride, benzoyl alcohol, meta-cresol, a combination thereof, or other preservatives. In several embodiments, the preservative is selected from the group consisting chlorobutanol, alcohol, benzalkonium chloride, benzethonium chloride, benzoic acid, benzyl alcohol, boric acid, bronopol, butylated hydroxyanisole (BHA), butylene glycol, butylparaben, calcium acetate, calcium chloride, calcium lactate, carbon dioxide, bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, magnesium trisilicate, isopropyl alcohol, lactic acid, methylparaben, monothioglycerol, parabens (methyl, ethyl and propyl), pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, propylparaben, propylparaben sodium, sodium acetate, sodium benzoate, sodium borate, sodium lactate, sodium metabisulfite, sodium propionate, sodium sulfite, sorbic acid, sulfobutyletherb-cyclodextrin, sulfur dioxide, edetic acid, thimerosal, xylitol, and/or combinations of any of the foregoing.

In several embodiments, the preservative is present in the composition at a concentration of 1.0 mg/mL to 9.0 mg/mL, or any concentration range subsumed therein, including but not limited to, 3.0 mg/mL to 8.0 mg/mL, 4.0 mg/mL to 7.0 mg/mL, 4.5 mg/mL to 6.5 mg/mL, 4.0 mg/mL to 6.0 mg/mL, or 5.0 mg/mL to 6.0 mg/mL. In several embodiments, the pharmaceutical formulation includes a preservative (or one or more preservatives) at a concentration of equal to or less than about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, or ranges including and/or spanning the aforementioned values.

In several embodiments, the pharmaceutical formulations include chlorobutanol (or a chlorobutanol source) at a concentration of 1.0 mg/mL to 9.0 mg/mL, or any concentration range subsumed therein, including but not limited to, 3.0 mg/mL to 8.0 mg/mL, 4.0 mg/mL to 7.0 mg/mL, 4.5 mg/mL to 6.5 mg/mL, 4.0 mg/mL to 6.0 mg/mL, or 5.0 mg/mL to 6.0 mg/mL. In several embodiments, the pharmaceutical formulation includes chlorobutanol at a concentration of equal to or less than about: 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, or ranges including and/or spanning the aforementioned values. In several embodiments of the pharmaceutical formulations, the chlorobutanol source is chlorobutanol hemihydrate.

In several embodiments, the pharmaceutical formulation comprises a preservative (e.g., chlorobutanol, chlorobutanol hemihydrate, etc.) at a molarity of equal to or less than about: 0.007 M, 0.01 M, 0.012 M, 0.014 M, 0.016 M, 0.018 M, 0.019 M, 0.020 M, 0.022 M, 0.025 M, or ranges including and/or spanning the aforementioned values. For example, in several embodiments, the pharmaceutical formulation comprises a preservative (e.g., chlorobutanol, chlorobutanol hemihydrate, etc.) at a molarity ranging from 0.007 M to 0.022 M, 0.012 M to 0.020 M, 0.012 M to 0.018 M, 0.014 M to 0.019 M, etc.

In several embodiments of the pharmaceutical formulation, the preservative is chlorobutanol present in a concentration range of 4.0 mg/mL to 7.0 mg/mL. In other embodiments of the pharmaceutical formulations, the preservative is chlorobutanol present at a concentration of about 5.5 mg/mL.

Metal Complexing Agents and/or Stabilizing Agent

In several embodiments, the pharmaceutical formulation includes a metal complexing agent. In several embodiments, the metal complexing agent is ethylenediaminetetraacetic acid (EDTA), disodium edetate dihydrate (disodium EDTA), diethylenetriamine pentaacetic acid (DTPA), or any other suitable metal complexing agent, or a combination thereof, but the present disclosure is not limited thereto. In several embodiments, the pharmaceutical formulations include a metal complexing agent (e.g., EDTA, disodium EDTA, etc.) at a concentration of 0.01 mg/mL to 0.10 mg/mL, or any concentration range subsumed therein, including but not limited to, 0.01 mg/mL to 0.08 mg/mL, 0.01 mg/mL to 0.05 mg/mL, 0.01 mg/mL to 0.03 mg/mL, or 0.01 mg/mL to 0.02 mg/mL. In several embodiments, the pharmaceutical formulation includes a metal complexing agent (e.g., EDTA, disodium EDTA, etc.) at a concentration of equal to or less than about: 0.005 mg/mL, 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.10 mg/mL, or ranges including and/or spanning the aforementioned values.

In several embodiments of the pharmaceutical formulation, the metal complexing agent is disodium EDTA present in a concentration range of 0.005 mg/mL to 0.05 mg/mL. In several embodiments of the pharmaceutical formulations, the metal complexing agent is disodium EDTA present at a concentration of about 0.02 mg/mL.

In several embodiments, the pharmaceutical formulation comprises a metal complexing agent (e.g., EDTA, disodium EDTA etc.) at a molarity of equal to or less than about: 1×10⁻⁵ M, 2.5×10⁻⁵ M, 5.0×10⁻⁵ M, 6.0×10⁻⁵ M, 7.5×10⁻⁵ M, 1.0×10⁻⁴ M, or ranges including and/or spanning the aforementioned values. For example, in several embodiments, the pharmaceutical formulation comprises a metal complexing agent (e.g., EDTA, disodium EDTA etc.) at a molarity ranging from 1×10⁻⁵ M to 1×10⁻⁴ M, 5.0×10⁻⁵ M to 6.0×10⁻⁵ M, etc.

### Tonicity Agents

In several embodiments, the pharmaceutical formulation comprises one or more tonicity agents. In several embodiments, the tonicity agent may include or is sodium chloride, dextrose, glucose, glycerin, cellulose, mannitol, polysorbate, propylene glycol, sodium iodide, or a combination thereof, but the present disclosure is not limited thereto. In several embodiments, the tonicity agent is present at a concentration of 1.0 mg/mL to 5.0 mg/mL, or any concentration range subsumed therein, including but not limited to, 1.0 mg/mL to 4.0 mg/mL, 1.0 mg/mL to 3.0 mg/mL, 2.0 mg/mL to 5.0 mg/mL, 2.0 mg/mL to 4.0 mg/mL. In several embodiments, the tonicity agent is present a concentration of equal to or less than about: 0.5 mg/mL, 1.0 mg/mL, 2.0 mg/mL, 3.0 mg/mL, 4.0 mg/mL, 5.0 mg/mL, or ranges including and/or spanning the aforementioned values.

In several embodiments of the pharmaceutical formulation, the tonicity agent is sodium chloride and is present at a concentration of 1.0 mg/mL to 5.0 mg/mL, or any concentration range subsumed therein, including but not limited to, 1.0 mg/mL to 4.0 mg/mL, 1.0 mg/mL to 3.0 mg/mL, 2.0 mg/mL to 5.0 mg/mL, 2.0 mg/mL to 4.0 mg/mL. In several embodiments, the pharmaceutical formulations include sodium chloride present a concentration of equal to or less than about: 0.5 mg/mL, 1.0 mg/mL, 2.0 mg/mL, 3.0 mg/mL, 4.0 mg/mL, 5.0 mg/mL, or ranges including and/or spanning the aforementioned values.

In several embodiments of the pharmaceutical formulation, the tonicity agent is sodium chloride present in a concentration range of 1.0 mg/mL to 5.0 mg/mL. In several embodiments of the pharmaceutical formulation, the tonicity agent is sodium chloride present at a concentration of about 1.50 mg/mL, 1.75 mg/mL, 2.00 mg/mL, 2.25 mg/mL, 2.50 mg/mL, 2.75 mg/mL or 3.00 mg/mL.

In several embodiments, the pharmaceutical formulation comprises the tonicity agent at a molarity of equal to or less than about: 0.01 M, 0.02 M, 0.04 M, 0.05 M, 0.06 M, 0.07 M, 0.080 M, 0.10 M, or ranges including and/or spanning the aforementioned values. For example, in several embodiments, the pharmaceutical formulation comprises the tonicity agent at a molarity ranging from 0.01 M to 0.10 M, 0.02 M to 0.04 M, 0.01 M to 0.05 M, etc.

### Antioxidants

In several embodiments, the pharmaceutical formulation comprises an antioxidant. In several embodiments, the antioxidant is selected from the group consisting of sodium metabisulfite, sodium bisulfate, other sulfites, butylated hydroxytoluene, tocopherol, or a combination thereof, but the present disclosure is not limited thereto. In several embodiments, the pharmaceutical formulations include the antioxidant at a concentration of 0.1 mg/mL to 1.0 mg/mL, or any concentration range subsumed therein, including but not limited to, 0.1 mg/mL to 0.9 mg/mL, 0.1 mg/mL to 0.8 mg/mL, 0.1 mg/mL to 0.5 mg/mL, 0.2 mg/mL to 0.5 mg/mL, or 0.2 mg/mL to 0.4 mg/mL. In several embodiments, the pharmaceutical formulations includes the antioxidant at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, or ranges including and/or spanning the aforementioned values.

In several embodiments, the pharmaceutical formulations include sodium metabisulfite present at a concentration of 0.1 mg/mL to 1.0 mg/mL, or any concentration range subsumed therein, including but not limited to, 0.1 mg/mL to 0.9 mg/mL, 0.1 mg/mL to 0.8 mg/mL, 0.1 mg/mL to 0.5 mg/mL, 0.2 mg/mL to 0.5 mg/mL, or 0.2 mg/mL to 0.4 mg/mL. In several embodiments, the pharmaceutical formulations include sodium metabisulfite present at a concentration of equal to or less than about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1.0 mg/mL, or ranges including and/or spanning the aforementioned values.

In several embodiments of the pharmaceutical formulation, the antioxidant is sodium metabisulfite present in a concentration range of 0.5 mg/mL to 1.0 mg/mL. In several embodiments of the pharmaceutical formulations, the antioxidant is sodium metabisulfite present at a concentration of equal to or less than about: 0.50 mg/mL, 0.55 mg/mL, 0.60 mg/mL, 0.65 mg/mL, 0.70 mg/mL, 0.75 mg/mL, 0.80 mg/mL, 0.85 mg/mL, 0.90 mg/mL, 0.95 mg/mL, or 1.0 mg/mL, or ranges including and/or spanning the aforementioned values.

In several embodiments, the pharmaceutical formulation comprises the antioxidant at a molarity of equal to or less than about: 0.001 M, 0.002 M, 0.004 M, 0.005 M, 0.006 M, 0.007 M, 0.0080 M, 0.010 M, or ranges including and/or spanning the aforementioned values. For example, in several embodiments, the pharmaceutical formulation comprises the antioxidant at a molarity ranging from 0.001 M to 0.010 M, 0.002 M to 0.004 M, 0.001 M to 0.005 M, etc.

### pH Adjustor

In several embodiments of the pharmaceutical formulations, the formulations include water. In other embodiments of the pharmaceutical formulations, other suitable solvents may be included, such as an alcohol solvent or other organic solvents, in addition to or instead of water. In several embodiments, the pharmaceutical formulations include a pH adjustor, such as hydrochloric acid (HCl), sodium hydroxide (NaOH), acetic acid, ascorbic acid, sulphuric acid, tartaric acid, or a combination thereof. In several embodiments of the pharmaceutical formulations, the pH adjustor includes 10% HCl and as needed, NaOH.

### IN Delivery Using a Nasal Spray

The disclosed IN pharmaceutical formulations can be administered by IN delivery using a nasal spray. Nasal sprays facilitate IN delivery of an API pharmaceutical formulations to one or more nostrils of a human patient. A nasal spray has a spray pump for discharging a dose volume of the pharmaceutical formulation in a single spray to a single nostril, or in two or more sprays to one or more nostrils. The dose volume of the IN pharmaceutical formulation contains the dose amount of the API.

In some embodiments, the nasal spray is a unit-dose nasal spray that administers a single dose volume of the pharmaceutical formulation in a single spray to a single nostril, or in two or more sprays to one or more nostrils, and such unit-dose nasal spray is disposed thereafter. In other embodiments, the nasal spray is a bi-dose nasal spray that can administer two dose volumes of the pharmaceutical formulation in two or more sprays to one or more nostrils, and such bi-dose nasal spray is disposed thereafter. In some embodiments, the unit-dose nasal spray or the bi-dose nasal spray is pre-primed to provide accurate dosing and ready to use capability. In still other embodiments, the nasal spray can administer three or more dose volumes of the pharmaceutical formulation.

In some embodiments, the dose volume of the IN pharmaceutical formulation is from 0.01 mL to 0.30 mL. In other embodiments, the dose volume of the IN pharmaceutical formulation is from 0.05 mL to 0.15 mL. In still other embodiments, the dose volume of the IN pharmaceutical formulation is about 0.10 mL. The dose volume is the volume that contains the dose amount of the API. The dose amount of the API will vary depending on the particular API because different APIs will have different therapeutically effective amounts of dose. For instance, if the API is naloxone, then the dose amount of naloxone can be in a range of 0.5 mg to 20.0 mg, or any amount range subsumed therein. Alternatively, if the API is epinephrine, then the dose amount of epinephrine can be in a range of 0.1 mg to 5.0 mg, or any amount range subsumed therein.

Further, in some embodiments, the dose amount of the API can be discharged in one or more nasal sprays. Thus, in some embodiments, the dose amount of the API is discharged in a single spray. In other embodiments, the dose amount of the API is discharged in two or more sprays.

In some embodiments, the dose amount of the bile acid, or the salt thereof (such as STC), is in a range of 0.1 mg to 1.5 mg, or any amount range subsumed therein, including but not limited to, 0.5 mg to 1.1 mg, 0.6 mg to 1.3 mg, 0.7 mg to 1.2 mg, 0.75 mg to 0.95 mg, 0.75 mg to 0.85 mg, 0.7 mg to 0.9 mg, or 0.7 mg to 0.8 mg. In other embodiments, the dose amount of the bile acid, or the salt thereof (such as STC), is in an amount of 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, or 1.5 mg. To illustrate, if the IN pharmaceutical formulation has a bile acid, or a salt thereof (such as STC), in a concentration of about 8 mg/mL and the dose volume is about 0.1 mL, then the dose amount of the bile acid, or the salt thereof (such as STC), would be about 0.8 mg. In several embodiments, a single spray from the dispensing device provides a dose of bile acid, or the salt thereof (such as STC) in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, a single spray from the dispensing device provides a dose of bile acid, or the salt thereof (such as STC) in an amount of equal to or less than about: 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1.0 mg, 1.05 mg, 1.1 mg, 1.15 mg, 1.2 mg, 1.25 mg, 1.3 mg, 1.35 mg, 1.4 mg, 1.45 mg, 1.5 mg, or ranges including and/or spanning the aforementioned values.

In several embodiments, the dose amount of the API, or the pharmaceutically acceptable salt thereof, is in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, the dose amount of the API, or the pharmaceutically acceptable salt thereof, is in an amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2. mg, 2.3 mg, 2.4 mg, 2.5 mg, or ranges including and/or spanning the aforementioned values. To illustrate, if the IN API pharmaceutical formulation has an API concentration of about 8 mg/mL and the dose volume is about 0.1 mL, then the dose amount of the API, or a pharmaceutically acceptable salt thereof, would be about 0.8 mg. In several embodiments, a single spray from the dispensing device provides a dose of API, or the pharmaceutically acceptable salt thereof, in a range of 0.1 mg to 2.5 mg, or any amount range subsumed therein, including but not limited to, 0.5 mg to 1.5 mg, 0.75 mg to 1.25 mg, 0.8 mg to 1.2 mg, 0.9 mg to 1.1 mg, 0.95 mg to 1.05 mg. In several embodiments, a single spray from the dispensing device provides a dose of API, or the pharmaceutically acceptable salt thereof, in an amount of equal to or less than about: 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2. mg, 2.3 mg, 2.4 mg, 2.5 mg, or ranges including and/or spanning the aforementioned values.

### Method of Providing a Rapid Absorption of an API Using IN Formulations for Treatments or Indications

The disclosed pharmaceutical formulations can provide a rapid delivery of API into the bloodstream of a human patient by IN delivery, comparable to API IM auto-injectors. Surprisingly, as disclosed elsewhere herein, the compositions disclosed herein can provide delivery that is more rapid that other delivery systems (e.g., has a lower tₘₐₓ, higher AUC_{0-t*}, AUC_{0-10 min}, AUC_{0-30 min}, etc.) than other delivery systems, including IM or other IN compositions. As described herein, a bile salt, such as STC, can enhance the absorption of the API via the nasal mucosa, and into the bloodstream. The rapid delivery is a desired feature because of disclosed formulations potential use as an emergency treatment.

Accordingly, disclosed are methods of providing a rapid delivery of API to a human patient, the method comprising the step of administrating a dose amount of API from any of the disclosed pharmaceutical formulations to at least one nostril of a human patient to treat a condition, wherein the administrating is by intranasal (IN) delivery using a nasal spray, and wherein post-administration of the pharmaceutical formulation by IN delivery, a Cₘₐₓ of 5 ng/mL to 15 ng/mL and a tₘₐₓ of less than 15 minutes are achieved.

In several embodiments, as disclosed herein, methods of treating a condition are provided. In several embodiments, the method comprises identifying a patient (e.g., a human patient in need of treatment). In several embodiments, the patient in need of treatment is a patient suffering from a condition or at risk of suffering from a condition as disclosed elsewhere herein. In several embodiments, the method comprises administering a dose of a formulation as described herein to the patient. In several embodiments, the dose is provided as one or more sprays from a dispensing device. In several embodiments, the dose is delivered to the nostril of the patient (or both nostrils).

In several embodiments, the condition is a type-I hypersensitivity reaction (systemic allergic reaction), an acute asthmatic attack, cardiac arrest, Stokes-Adams Syndrome, or a combination of the foregoing In several embodiments, the condition is an allergic reaction, such as Type 1 allergic reactions. In several embodiments, the condition is a type-I hypersensitivity reaction (systemic allergic reaction), an acute asthmatic attack, cardiac arrest, Stokes-Adams Syndrome, or a combination of the foregoing. Anaphylaxis is an example of a Type 1 allergic reaction. In other embodiments, the condition is hypotension associated with septic shock, or for increasing mean arterial blood pressure in a patient with hypotension associated with septic shock. In several embodiments, the type-I hypersensitivity reaction is selected from allergic asthma, allergic conjunctivitis, allergic rhinitis, anaphylaxis, angioedema, urticaria, eosinophilia, drug allergy, and food allergy. In several embodiments, the condition is an emergency condition. In several embodiments, the condition includes bronchospasm, sensitivity reactions, cardiopulmonary resuscitation, cardiac arrhythmias, local vasoconstriction, premature labor, hypoglycemia, gastrointestinal hemorrhage, renal hemorrhage, bleeding, or mydriasis during intraocular surgery. In several embodiments, the pharmaceutical formulation is used in a method of increasing mean arterial blood pressure in patients with hypotension associated with septic shock, to relieve respiratory distress due to bronchospasm, to provide rapid relief of hypersensitivity reactions to drugs and other allergens, to prolong the action of infiltration anesthetics, and/or combinations thereof.

In several embodiments, the disclosed IN pharmaceutical formulations can achieve an AUC_{0-10 min}, AUC_{0-30 min}, AUC_{0-180 min}, AUC_{0-X min}, Cₘₐₓ, tₘₐₓ, and bioavailability, including relative bioavailability (RBA), similar to that of IM API auto-injectors (e.g. 1 mg/mL IM API injector) at a similar rate, or a similar time period, as that of the IM API auto-injector. In other embodiments, the disclosed IN pharmaceutical formulations can achieve an AUC_{0-10 min}, AUC_{0-30 min}, AUC_{0-180 min}, Cₘₐₓ, and bioavailability similar to that of an IM API auto-injectors (e.g. 1 mg/mL IM API auto-injector) at a faster rate, or a shorter time period, compared to that of the IM API auto-injector. An example of a 1 mg/mL IM API auto-injector is an EpiPen® (0.3 mg epinephrine).

In several embodiments, an IN composition as disclosed herein achieves a Cₘₐₓ of greater than or equal to about: 100 pg/mL, 200 pg/mL, 300 pg/mL, 350 pg/mL, 400 pg/mL, 450 pg/mL, 500 pg/mL, 550 pg/mL, 600 pg/mL, 650 pg/mL, or ranges including and/or spanning the aforementioned values. For example, in several embodiments, an IN composition as disclosed herein achieves a Cₘₐₓ ranging from 100 pg/mL to 650 pg/mL, 300 pg/mL to 650 pg/mL, 350 pg/mL to 600 pg/mL, 300 pg/mL to 650 pg/mL, 400 pg/mL to 650 pg/mL, 450 pg/mL to 600 pg/mL, etc. In several embodiments, the Cₘₐₓ is measured as the geometric mean of a representative patient population. In several embodiments, the Cₘₐₓ is measured as the arithmetic mean of a representative patient population. In several embodiments, the Cₘₐₓ for the IN composition differs from the Cₘₐₓ for an IM formulation API by less than or equal to about: 40%, 30%, 20%, 10%, 5%, or ranges including and/or spanning the aforementioned values.

In several embodiments, an IN composition as disclosed herein achieves a tₘₐₓ (in minutes) of less than or equal to about: 5, 6, 7, 8, 9, 10, 11, 12, 13, 14, 15, 17.5, 20, 25, or ranges including and/or spanning the aforementioned values. For example, in several embodiments, an IN composition as disclosed herein achieves a tₘₐₓ (in minutes) ranging from 5 to 15, 7 to 10, 6 to 12, 5 to 10, 5 to 20, etc. In several embodiments, the tₘₐₓ is measured as the geometric mean of a representative patient population. In several embodiments, the tₘₐₓ is measured as the arithmetic mean of a representative patient population. In several embodiments, the tₘₐₓ for the IN composition differs from the tₘₐₓ for an IM formulation API by less than or equal to about: 40%, 30%, 20%, 10%, 5%, or ranges including and/or spanning the aforementioned values.

In several embodiments, an IN composition as disclosed herein achieves a AUC_{0-t*} of greater than or equal to about: 10 pg/mL*hr, 15 pg/mL*hr, 20 pg/mL*hr, 25 pg/mL*hr, 26 pg/mL*hr, 27 pg/mL*hr, 28 pg/mL*hr, 29 pg/mL*hr, 30 pg/mL*hr, 32 pg/mL*hr, 35 pg/mL*hr, 40 pg/mL*hr, 45 pg/mL*hr, 50 pg/mL*hr, or ranges including and/or spanning the aforementioned values. For example, in several embodiments, an IN composition as disclosed herein achieves a AUC_{0-t*} ranging from 10 pg/mL*hr to 35 pg/mL*hr, 15 pg/mL*hr to 30 pg/mL*hr, 25 pg/mL*hr to 35 pg/mL*hr, 30 pg/mL*hr to 35 pg/mL*hr, 28 pg/mL*hr to 35 pg/mL*hr, 20 pg/mL*hr to 40 pg/mL*hr, etc. In several embodiments, the AUC_{0-t*} is measured as the geometric mean of a representative patient population. In several embodiments, the AUC_{0-t*} is measured as the arithmetic mean of a representative patient population. In several embodiments, the AUC_{0-t*} for the IN composition differs from the AUC_{0-t*} for an IM formulation API by less than or equal to about: 40%, 30%, 20%, 10%, 5%, or ranges including and/or spanning the aforementioned values.

In several embodiments, an IN composition as disclosed herein achieves a AUC_{0-10 min} of greater than or equal to about: 10 pg/mL*hr, 15 pg/mL*hr, 20 pg/mL*hr, 25 pg/mL*hr, 30 pg/mL*hr, 35 pg/mL*hr, 40 pg/mL*hr, 45 pg/mL*hr, 50 pg/mL*hr, 55 pg/mL*hr, 65 pg/mL*hr, or ranges including and/or spanning the aforementioned values. For example, in several embodiments, an IN composition as disclosed herein achieves a AUC_{0-10 min} ranging from 20 pg/mL*hr to 50 pg/mL*hr, 10 pg/mL*hr to 60 pg/mL*hr, 25 pg/mL*hr to 55 pg/mL*hr, 40 pg/mL*hr to 50 pg/mL*hr, 45 pg/mL*hr to 60 pg/mL*hr, 20 pg/mL*hr to 60 pg/mL*hr, etc. In several embodiments, the AUC_{0-10 min} is measured as the geometric mean of a representative patient population. In several embodiments, the AUC_{0-10 min} is measured as the arithmetic mean of a representative patient population. In several embodiments, the AUC0-10 min for the IN composition differs from the AUC0-10 min for an IM formulation API by less than or equal to about: 40%, 30%, 20%, 10%, 5%, or ranges including and/or spanning the aforementioned values.

In several embodiments, an IN composition as disclosed herein achieves a AUC_{0-30 min} of greater than or equal to about: 30 pg/mL*hr, 40 pg/mL*hr, 50 pg/mL*hr, 60 pg/mL*hr, 70 pg/mL*hr, 80 pg/mL*hr, 90 pg/mL*hr, 100 pg/mL*hr, 110 pg/mL*hr, 120 pg/mL*hr, 130 pg/mL*hr, 140 pg/mL*hr, 150 pg/mL*hr, 160 pg/mL*hr, 170 pg/mL*hr, or ranges including and/or spanning the aforementioned values. For example, in several embodiments, an IN composition as disclosed herein achieves a AUC_{0-30 min} ranging from 90 pg/mL*hr to 140 pg/mL*hr, 100 pg/mL*hr to 160 pg/mL*hr, 70 pg/mL*hr to 140 pg/mL*hr, 120 pg/mL*hr to 140 pg/mL*hr, 60 pg/mL*hr to 160 pg/mL*hr, 130 pg/mL*hr to 140 pg/mL*hr, etc. In several embodiments, the AUC_{0-30 min} is measured as the geometric mean of a representative patient population. In several embodiments, the AUC_{0-30 min} is measured as the arithmetic mean of a representative patient population.

In several embodiments, an IN composition as disclosed herein achieves a AUC_{0-6 hrs} of greater than or equal to about: 100 pg/mL*hr, 200 pg/mL*hr, 250 pg/mL*hr, 300 pg/mL*hr, 325 pg/mL*hr, 350 pg/mL*hr, 375 pg/mL*hr, 400 pg/mL*hr, 425 pg/mL*hr, 450 pg/mL*hr, 475 pg/mL*hr, 500 pg/mL*hr, 550 pg/mL*hr, or ranges including and/or spanning the aforementioned values. For example, in several embodiments, an IN composition as disclosed herein achieves a AUC_{0-6 hrs} ranging from 300 pg/mL*hr to 500 pg/mL*hr, 250 pg/mL*hr to 350 pg/mL*hr, 300 pg/mL*hr to 450 pg/mL*hr, 250 pg/mL*hr to 500 pg/mL*hr, 100 pg/mL*hr to 550 pg/mL*hr, 425 pg/mL*hr to 475 pg/mL*hr, etc. In several embodiments, the AUC_{0-6 hrs} is measured as the geometric mean of a representative patient population. In several embodiments, the AUC_{0-6 hrs} is measured as the arithmetic mean of a representative patient population.

In several embodiments, an IN composition as disclosed herein achieves a AUC_{0-∞} of greater than or equal to about: 100 pg/mL*hr, 200 pg/mL*hr, 250 pg/mL*hr, 300 pg/mL*hr, 325 pg/mL*hr, 350 pg/mL*hr, 375 pg/mL*hr, 400 pg/mL*hr, 425 pg/mL*hr, 450 pg/mL*hr, 475 pg/mL*hr, 500 pg/mL*hr, 550 pg/mL*hr, 600 pg/mL*hr, or ranges including and/or spanning the aforementioned values. For example, in several embodiments, an IN composition as disclosed herein achieves a AUG_{0-∞} ranging from 300 pg/mL*hr to 550 pg/mL*hr, 250 pg/mL*hr to 600 pg/mL*hr, 350 pg/mL*hr to 550 pg/mL*hr, 500 pg/mL*hr to 550 pg/mL*hr, 100 pg/mL*hr to 600 pg/mL*hr, 375 pg/mL*hr to 550 pg/mL*hr, etc. In several embodiments, the AUC_{0-∞} is measured as the geometric mean of a representative patient population. In several embodiments, the AUC_{0-∞} is measured as the arithmetic mean of a representative patient population.

In several embodiments, the IN pharmaceutical formulations can achieve a Cₘₐₓ in a range of 5 ng/mL to 15 ng/mL, or any range subsumed therein, including but not limited to 5 ng/mL to 10 ng/mL, 7 ng/mL to 14 ng/mL, 8 ng/mL to 13 ng/mL, 10 ng/mL to 15 ng/mL, or 11 ng/mL to 15 ng/mL. In other embodiments, the Cₘₐₓ is about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, or about 15 ng/mL. By comparison, an IM API auto-injector, such as a 1 mg/mL IM API auto-injector can achieve a Cₘₐₓ of 12.1 ng/mL.

In several embodiments, the IN pharmaceutical formulations can achieve a tₘₐₓ in less than 25 minutes (or any range subsumed therein), including, but not limited to, less than 24, 23, 22, 21, 20, 19, 18, 17, 16, 15, 14, 13, 12, 11, 10, 9, 8, 7, 6, 5, 4, 3, 2, or 1 minute(s).

In several embodiments, the IN pharmaceutical formulations can achieve a 100% relative bioavailability of API with respect to a 1 mg/mL IM API auto-injector. In other embodiments, the IN pharmaceutical formulations can achieve a relative bioavailability of API of 75% to 125% (or any range subsumed therein) with respect to a 1 mg/mL IM API auto-injector.

In several embodiments, the IN pharmaceutical formulations can achieve an AUC_{0-10 min} in a range of 50 (ng*min)/mL to 80 (ng*min)/mL, or any range subsumed therein, including, but not limited to, 55 (ng*min)/mL to 65 (ng*min)/mL, 60 (ng*min)/mL to 70 (ng*min)/mL, or 65 (ng*min)/mL to 75 (ng*min)/mL. In other embodiments, the IN pharmaceutical formulations can achieve an AUC_{0-10 min} of at least 50 (ng*min)/mL, 55 (ng*min)/mL, 60 (ng*min)/mL, 65 (ng*min)/mL, 70 (ng*min)/mL, 75 (ng*min)/mL, or 80 (ng*min)/mL. By comparison, an IM API auto-injector, such as a 1 mg/mL IM API auto-injector, can achieve an AUC_{0-10 min} of 64 (ng*min)/mL.

In several embodiments, the IN pharmaceutical formulations can achieve an AUC_{0-30 min} in a range of 100 (ng*min)/mL to 170 (ng*min)/mL or any range subsumed therein, including, but not limited to, 115 (ng*min)/mL to 135 (ng*min)/mL, 115 (ng*min)/mL to 130 (ng*min)/mL, or 120 (ng*min)/mL to 130 (ng*min)/mL. In other embodiments, the IN pharmaceutical formulations can achieve an AUC_{0-30 min} of at least 110 (ng*min)/mL, 115 (ng*min)/mL, 120 (ng*min)/mL, 125 (ng*min)/mL, 130 (ng*min)/mL, 135 (ng*min)/mL, or 140 (ng*min)/mL. By comparison, an IM API auto-injector, such as a 1 mg/mL IM API auto-injector can achieve an AUC_{0-30 mins} of 133 (ng*min)/mL.

In several embodiments, the IN pharmaceutical formulations can achieve an AUC_{0-180 min} in a range of 150 (ng*min)/mL to 300 (ng*min)/mL or any range subsumed therein, including, but not limited to, 150 (ng*min)/mL to 275 (ng*min)/mL, 150 (ng*min)/mL to 250 (ng*min)/mL, 150 (ng*min)/mL to 225 (ng*min)/mL, 150 (ng*min)/mL to 200 (ng*min)/mL, 175 (ng*min)/mL to 275 (ng*min)/mL, 175 (ng*min)/mL to 250 (ng*min)/mL, 175 (ng*min)/mL to 225 (ng*min)/mL, 175 (ng*min)/mL to 200 (ng*min)/mL, 200 (ng*min)/mL to 300 (ng*min)/mL, 200 (ng*min)/mL to 275 (ng*min)/mL, or 200 (ng*min)/mL to 250 (ng*min)/mL. In other embodiments, the IN pharmaceutical formulations can achieve an AUC_{0-180 min} of at least 150 (ng*min)/mL, 160 (ng*min)/mL, 170 (ng*min)/mL, 180 (ng*min)/mL, 190 (ng*min)/mL, 200 (ng*min)/mL, 210 (ng*min)/mL, 220 (ng*min)/mL, 230 (ng*min)/mL, 240 (ng*min)/mL, 250 (ng*min)/mL, 260 (ng*min)/mL, 270 (ng*min)/mL, 280 (ng*min)/mL, 290 (ng*min)/mL, or 300 (ng*min)/mL.

In several embodiments, if the bile acid, or the salt thereof, causes decreased cilia in a respiratory epithelium of the human subject, then such decreased cilia is substantially reversed within 7 days, including but not limited to, within 6 days, within 5 days, within 4 days, within 3 days, within 2 days, or within 1 day. In other embodiments, if the bile acid, or the salt thereof, causes hyperplasia of a respiratory epithelium of the human subject, then such hyperplasia is substantially reversed within 7 days, including but not limited to, within 6 days, within 5 days, within 4 days, within 3 days, within 2 days, or within 1 day. In still other embodiments, if the bile acid, or the salt thereof, causes decreased cilia and hyperplasia of a respiratory epithelium of the human subject, then such decreased cilia and hyperplasia are substantially reversed within 7 days, including but not limited to, within 6 days, within 5 days, within 4 days, within 3 days, within 2 days, or within 1 day. In further embodiments, if the bile acid, or the salt thereof, causes any change to a nasal mucosa of the human subject, then such change is substantially reversed within 7 days, including but not limited to, within 6 days, within 5 days, within 4 days, within 3 days, within 2 days, or within 1 day.

In several embodiments, surprisingly, despite having one or more of a higher total dose, Cₘₐₓ, AUC_{0-10 min}, AUC_{0-30 min}, AUC_{0-180 min}, AUC_{0-6 hr}, and/or AUC_{0-∞} than a comparator IM administration of API (e.g., provided from an EpiPen®), a dose of the formulation disclosed herein causes a number of milder side effects (e.g., adverse events) than the IM dose. In several embodiments, surprisingly, despite having a lower tₘₐₓ than a comparator IM administration of API, a dose of the formulation disclosed herein causes a number of milder side effects (e.g., adverse events) than the IM dose. In several embodiments, the milder side effects are one or more of nausea, vomiting, tachycardia, bradycardia, tremor, diastolic hypertension, hypotension, tachypnea, or combinations of the foregoing. In several embodiments, the incidence for of adverse events, as disclosed herein, for IN composition is less than that for an IM formulation API by equal to or at least about: 40%, 30%, 20%, 10%, 5%, or ranges including and/or spanning the aforementioned values.

Surprisingly, it has been found that several embodiments of the IN formulations disclosed herein have less incidences of mucosal edema, rhinorrhea, nasal discharge, and/or nasal discomfort at an increased concentration (e.g., over 8 mg/mL) of the enhancing agent. In several embodiments, the incidence for of mucosal edema, rhinorrhea, nasal discharge, and/or nasal discomfort is reduced by equal to or at least about: 40%, 30%, 20%, 10%, 5%, or ranges including and/or spanning the aforementioned values. In several embodiments, surprisingly, at an increased concentration (e.g., over 8 mg/mL) of the enhancing agent, the incidents of severe events and/or grade 3 occur based on a Nasal and Oropharyngeal Mucosa Exam (NOME) scale does not increase. In several embodiments, surprisingly, at an increased concentration (e.g., over 8 mg/mL) of the enhancing agent, the incidents of severe events and/or grade 3 occur based on a Self-Reported Nasal Symptoms (SRNS) scale does not increase. In several embodiments, surprisingly, a population of subjects experiences higher rates of normosmia 6 hours after an IN dose as measured by the University of Pennsylvania Smell Identification Test (UPSIT).

Advantageously, in some embodiments of the pharmaceutical formulations or corresponding methods, if the bile acid, or the salt thereof, causes decreased cilia in a respiratory epithelium of the human subject, then such decreased cilia is substantially reversed within 7 days, including but not limited to, within 6 days, within 5 days, within 4 days, within 3 days, within 2 days, or within 1 day. In other embodiments, if the bile acid, or the salt thereof, causes hyperplasia of a respiratory epithelium of the human subject, then such hyperplasia is substantially reversed within 7 days, including but not limited to, within 6 days, within 5 days, within 4 days, within 3 days, within 2 days, or within 1 day. In still other embodiments, if the bile acid, or the salt thereof, causes decreased cilia and hyperplasia of a respiratory epithelium of the human subject, then such decreased cilia and hyperplasia are substantially reversed within 7 days, including but not limited to, within 6 days, within 5 days, within 4 days, within 3 days, within 2 days, or within 1 day.

In further embodiments, if the bile acid, or the salt thereof, causes any change to a nasal mucosa of the human subject, then such change is substantially reversed within 7 days, including but not limited to, within 6 days, within 5 days, within 4 days, within 3 days, within 2 days, or within 1 day. In further embodiments, if the bile acid, or the salt thereof, causes any change to the ciliotoxicity of the human subject, then such change is substantially reversed within 7 days, including but not limited to, within 6 days, within 5 days, within 4 days, within 3 days, within 2 days, or within 1 day.

### EXAMPLES

### Example 1—Bile Salt Enhances Intranasal (IN) Absorption of API

Example 1 presents an animal study that demonstrates that bile salts can enhance the IN absorption of an API. In Example 1, STC (hydrate) is the exemplary bile salt used, and is the exemplary API used. Other suitable bile salts and APIs may also be utilized. The Example 1 pharmaceutical formulations tested are detailed in Tables 1.1-1.2. For brevity throughout this disclosure, “Epi” or “epi” refers to epinephrine, and “Conc.” refers to concentration.

TABLE 1.1
Formulations Tested (Excipients Not Shown)

| | | | | Bile Salt | |
|---|---|---|---|---|---|
| Delivery | Formulation | | Epi Conc. | Conc. (STC) | Volume |
| Route | No. | Arm Code | (mg/mL) | (mg/mL) | (μL) |
| IM | 0 | M | 1 | 0 | 25 |
| IN | 1 | T2a | 2 | 4 | 25 |
| | 2 | T2b | 2 | 6 | 25 |
| | 3 | T2c | 2 | 8 | 25 |
| | 4 | T2d | 2 | 10 | 25 |
| | 5 | T1 | 4 | 0 | 25 |
| | 6 | T3a | 4 | 4 | 25 |
| | 7 | T3b | 4 | 6 | 25 |
| | 8 | T3c | 4 | 8 | 25 |
| | 9 | T3d | 4 | 10 | 12.5 |
| | 10 | T4a | 8 | 4 | 25 |
| | 11 | T4b | 8 | 6 | 25 |
| | 12 | T4c | 8 | 8 | 12.5 |
| | 13 | T5 | 10 | 6 | 12.5 |

TABLE 1.2
Dose of Formulations Tested (Excipients Not Shown)

| | | | | | | Dose of | |
|---|---|---|---|---|---|---|---|
| | | Mean | | | | Bile | Num- |
| For- | | Body | | | | Salt | ber |
| mulation | Arm | Weight | Dose of Epi | | | (STC) | of |
| No. | Code | (kg) | d, mg | mg/kg | MRDH | (kg/mg) | rats (n) |
| 0 | M | 0.30 | 0.025 | 0.08 | 8 | 0.00 | 20 |
| 1 | T2a | 0.26 | 0.05 | 0.19 | 19 | 0.39 | 20 |
| 2 | T2b | 0.27 | 0.05 | 0.19 | 19 | 0.56 | 20 |
| 3 | T2c | 0.30 | 0.05 | 0.17 | 17 | 0.67 | 20 |
| 4 | T2d | 0.31 | 0.05 | 0.16 | 16 | 0.81 | 20 |
| 5 | T1 | 0.23 | 0.1 | 0.43 | 43 | 0.00 | 19 |
| 6 | T3a | 0.24 | 0.1 | 0.41 | 41 | 0.41 | 20 |
| 7 | T3b | 0.30 | 0.1 | 0.33 | 33 | 0.49 | 20 |
| 8 | T3c | 0.28 | 0.1 | 0.36 | 36 | 0.73 | 20 |
| 9 | T3d | 0.30 | 0.05 | 0.17 | 17 | 0.42 | 20 |
| 10 | T4a | 0.33 | 0.2 | 0.60 | 60 | 0.30 | 20 |
| 11 | T4b | 0.34 | 0.2 | 0.60 | 60 | 0.45 | 15 |
| 12 | T4c | 0.36 | 0.1 | 0.28 | 28 | 0.28 | 19 |
| 13 | T5 | 0.36 | 0.1 | 0.28 | 28 | 0.21 | 20 |

The excipients for the IN formulations in Table 1.1 include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

In Table 1.2, MRDH is the Maximum Relative Dose for Human for EpiPen® 0.01 mg/kg. The dose for the EpiPen® is 0.3 mg for adults with a body weight of >30 kg. The relative dose for the EpiPen® will be 0.003-0.01 mg/kg if a body weight of 30 to 100 kg is used. Thus, the MRDH for the EpiPen® is 0.01 mg/kg. In this rat model PK study in Example 1, the rat IN dose is in the range of 0.16-0.6 mg/kg, i.e. 16 to 60 times of the MRDH for the EpiPen®, as also listed in Table 1.2. Also, the amount of STC delivered in this PK study in Example 1 is in the range of 0 to 0.81 mg/kg.

Additionally, an intramuscular (IM) injection formulation having 1 mg/mL epinephrine and no bile salt served as the reference control to the Table 1.1 formulations. This 1 mg/mL IM injection control has an Arm Code of “M” and is used as the baseline for determining relative bioavailabilities (RBA) for AUC_{0-30 min} AUC_{0-180 min}, Cₘₐₓ, and Bile Salt Enhancement Factor (EF), as will be shown in Table 1.3. More particularly, Example 1 assessed the dose-normalized relative bioavailability (DN-RBA), based on the ratio of the dose-normalized pharmacokinetic (PK) parameter by IN administration of the Table 1 formulations versus that of the 1 mg/mL IM injection control. The DN-RBA is defined as follows:

$${R}_{X} ( S ) = \frac{{X}_{IN} ( {d}_{IN} S ) \frac{1}{{d}_{IN}}}{{X}_{IM} ( {d}_{IM} ) \frac{1}{{d}_{IM}}}$$

where R_{X} is the DN-RBA for PK parameters X;
S is the concentration of Bile Salt (i.e. STC) used in the Table 1 formulations;
X are partial AUC, AUC_{0-30 min}, and AUC_{0-180 min}, or Cₘₐₓ;
d_{IM} and d_{IN} are doses delivered by IM and IN routes, respectively.

The Bile Salt EF_{0-30/0-180/Cmax} to the IN absorption of the API (i.e. epinephrine), based on the DN-RBA equation above, is defined as follows:

$$EF ( S ) = \frac{\overline{R} ( S )}{\overline{R} ( 0 )}$$

where R̅(S) is an average of DN-RBA for X (3 PK parameters: AUC_{0-30 min}, AUC_{0-180 min}, and Cₘₐₓ) by the IN route at a given Bile Salt concentration S.

R̅(0) has the same definition at S=0, by the IN route.

Also shown in Table 1, for IN Formulations 1-8, 10-11, 25 μL of the respective formulation were administered by IN to the rats. For IN Formulations 9, 12, 13, only 12.5 μL of the respective formulation were administered by IN to the rats due to higher concentrations of bile salts and API. Also, the 1 mg/mL IM injection control were administered to 20 rats.

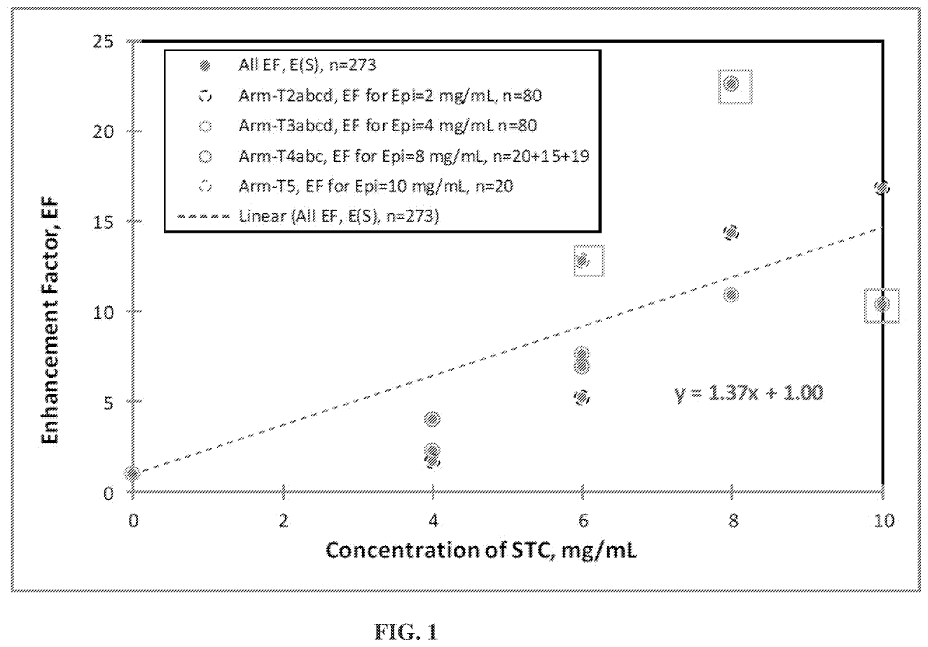

For the Bile Salt EF_{0-30/0-180/Cmax}, Formulation No. 5 served as R̅(0) because it has no STC. Table 1.3 and FIG. 1 show the results of the Bile Salt EF, which ranges from 1 to 23. Thus, Example 1 demonstrates that bile salt is advantageous as an absorption enhancer in IN delivery because it can provide an EF of up to 23. Notably, Formulation No. 12 having 8 mg/mL epinephrine and 8 mg/mL bile salt (STC) had the highest Bile Salt EF at 23. Formulation No. 12 also exhibited a DN-RBA, IN v. IM of about 88% for AUC_{0-30 min}, of about 61% for AUC_{0-180 min}, and of about 106% for Cₘₐₓ, as well as a mean RBA v. IM of about 85%. Also shown in Table 1.3 is the equation B=d×EF, where d=dose of the API (in mg), and EF is the Bile Salt EF. B characterizes the net IN absorption of the API (epinephrine). Notably, Formulation No. 12 scored the highest net IN absorption with a B value of about 2.3.

TABLE 1.3
Dose-Normalized Relative Bioavailability (DN-RBA), IN v. IM, and Bile Salt
Enhancement Factor (EF).

| | | Bile Salt | | | | | | |
|---|---|---|---|---|---|---|---|---|
| Formulation | | (STC) | Dose-Normalized Relative | | | | | |
| No. | | | Bioavailability (DN-RBA), IN | | | Mean | | |
| (From | Epi Conc. | Conc. | v. IM | | | RBA | Bile Salt | B = EF |
| Table 1) | (mg/mL) | (mg/mL) | AUC_{0-30 min} | AUC_{0-180 min} | Cₘₐₓ | v. IM | EF | *d |
| 1 | 2 | 4 | 6.7% | 6.6% | 5.0% | 6.1% | 1.6 | 0.1 |
| 2 | 2 | 6 | 22% | 16% | 21% | 20% | 5.2 | 0.3 |
| 3 | 2 | 8 | 58% | 45% | 59% | 54% | 14 | 0.7 |
| 4 | 2 | 10 | 67% | 47% | 77% | 63% | 17 | 0.8 |
| 5 | 4 | 0 | 2.3% | 6.4% | 2.6% | 3.8% | 1 | 0.1 |
| 6 | 4 | 4 | 7.2% | 13.5% | 4.8% | 8.5% | 2.3 | 0.2 |
| 7 | 4 | 6 | 30% | 24% | 32% | 29% | 7.6 | 0.8 |
| 8 | 4 | 8 | 44% | 35% | 45% | 41% | 11 | 1.1 |
| 9 | 4 | 10 | 40% | 28% | 50% | 39% | 10 | 0.5 |
| 10 | 8 | 4 | 15% | 19% | 11% | 15% | 4.0 | 0.8 |
| 11 | 8 | 6 | 28% | 26% | 24% | 26% | 6.9 | 1.4 |
| 12 | 8 | 8 | 88% | 61% | 106% | 85% | 23 | 2.3 |
| 13 | 10 | 6 | 48% | 33% | 63.3% | 48% | 12.8 | 1.3 |

FIG. 1 presents the Bile Salt EF results provided in Table 1.3. In particular, FIG. 1 is a graph of the Bile Salt (STC) Enhancement Factor as the Y-axis, entitled “Enhancement Factor, EF” versus Bile Salt (STC) concentration (mg/mL) as the X-axis, entitled “Concentration of STC, mg/mL.” The dotted line in FIG. 1 is the curve fitting based on the plots of the Bile Salt EF results in Table 1.3. In FIG. 1, the 3 data points marked with squares are the smaller IN volume (12.5 μL) due to higher concentrations of the bile salt and the API. Notably, as shown by y=1.37×+100 in FIG. 1, the Bile Salt EF had an approximate linear relationship with the Bile Salt (STC) concentration (mg/mL) in rats, in particular, EF≈1.37S+1, where EF_{0-30/0-180/Cmax} is the Bile Salt EF_{0-30/0-180/Cmax}, and S is the Bile Salt (STC) concentration. Also, FIG. 1 shows that Bile Salts (STC) can enhance the absorption of an API (epinephrine) even at higher API concentrations such as 8-10 mg/mL epinephrine, thus demonstrating that bile salts can be effective as an absorption enhancer for IN delivery of APIs.

### Example 2—General Toxicity Study of Bile Salts as Absorption Enhancer for IN Delivery

In Example 2, an animal study was conducted to determine the general toxicity of bile salts. In Example 2, STC (hydrate) is the exemplary bile salt used, and epinephrine is the exemplary API used. Other suitable bile salts and APIs may also be utilized. In this general toxicity study, a total of 220 rats were assessed based on Groups A1-A5 detailed below:

Group A1: negative control (saline), number of rats (n)=44;

Group A2: 1 mg/mL epinephrine, 0 mg/mL STC, n=44;

Group A3: 1 mg/mL epinephrine, 5 mg/mL STC, n=44;

Group A4: 1 mg/mL epinephrine, 10 mg/mL STC, n=44;

Group A5: 1 mg/mL epinephrine, 15 mg/mL STC, n=44.

In each of Groups A1-A5, there are 44 rats, for which 24 rats were assessed 1 day after the IN administration and 20 rats were assessed 14 days after the IN administration. The purpose of this study was to assess the general toxicity of a bile salt (STC) through IN delivery so that the concentration of the API (epinephrine) was fixed at a low concentration of 1 mg/mL. Each rat was treated with two (2) IN administrations of the respective formulations in Groups A1-A5. The time interval between the 2 IN treatments was 15 minutes. No rats died during this general toxicity study before they were sacrificed.

The excipients in Groups A2-A5 include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

The analysis of clinical pathology was conducted by a qualified veterinary laboratory. The clinical investigator concluded that there were no changes in hematology or clinical chemistry parameters observed on either Days 1 or 14, as compared to both saline and vehicle control groups (i.e. Groups A1-A2). In conclusion, two single IN administrations of the IN formulations in Groups A3-A5 between 15 minutes to rats did not result in changes in hematology or clinical chemistry parameters.

Histopathological evaluation was conducted by a qualified pathology laboratory. A total of 1,540 tissue samples from 220 rats, including (i) adrenal glands, (ii) brain, (iii) heart, (iv) kidneys, (v) liver, and (vi) lung lobes (left and right) were studied. The histopathological findings were graded from 1 to 5, with 1 as minimal, and 5 as severe, depending upon severity. The findings included an increased incidence of hemoglobin crystal/hemorrhage and an increased incidence of a mixed cell inflammation in the left and/or right lung lobes for the animals that were sacrificed on Day 1. The number of these changes observed was determined to be lower for the animals that were sacrificed on Day 14, showing recovery from the IN administration of the drugs.

The study concluded that the maximum tolerated dose was considered to be at the Group A5 level (1 mg/mL epinephrine, 15 mg/mL STC). Due to the minimal severity of the pulmonary findings, and that these minor findings did not result in clinical signs of toxicity, the No-Observed-Adverse-Effect Level (NOAEL) was also considered to be at the Group A5 level. Thus, advantageously, Example 2 demonstrates that bile salt can be safely used for clinical applications, even at high bile salt concentrations of 15 mg/mL.

The STC concentration in Group A5 is 15 mg/mL. Group A5 has 44 rats with an average body weight 0.298 kg on the treatment day. The NOAEL for the bile salt (STC) can be assessed as exceeding 15 mg/mL because 15 mg/mL was the highest studied concentration for IN administration. In this general safety study for STC, the highest dose of STC by an IN delivery is 0.75 mg (=15 mg/mL×0.025 mL×2), namely a relative dose of approximately 2.5 mg/kg (=0.75 mg/0.298 kg). Therefore, the NOAEL for bile salt (STC) at an IN delivery is 2.5 mg/kg or larger.

### Example 3—Local Irritation Study of Bile Salts as Absorption Enhancer for IN Delivery

In Example 3, an animal study was conducted to determine the local irritation of bile salts as an absorption enhancer for IN delivery, and more particularly, local irritation at the nasal mucosa. Example 3 investigates the histopathological effects of bile salt (STC) on nasal mucosa and damage reversibility in a rat model (n=378). Historically, bile salts have limited clinical use because of the irreversible damage to the mucosa and ciliotoxicity. Example 3 demonstrates that the damage for nasal mucosa is reversible in 3-7 days at high dose for IN delivery. In Example 3, STC (hydrate) is the exemplary bile salt used, and epinephrine is the exemplary API used. Other suitable bile salts and APIs may also be utilized.

In this study, a total of 378 rats were assessed based on Groups B1-B5 detailed in Table 3.1. Note that the “No.” columns in Tables 3.1-3.3 are the same.

TABLE 3.1
IN Formulations Tested for Nasal Mucosa Irritation Study.

| | | | | | API | Bile |
|---|---|---|---|---|---|---|
| | | | | | (Epi) | Salt |
| | | | Total number | | Conc. | Conc. |
| | | Arm | of sprays (K) (25 | B = d * | (mg/ | (STC) |
| No. | Group | Code | μL in each spray) | EF * K | mL) | (mg/mL) |
| 1 | B1 | B | 1 | N/A | 0 | 0 |
| 2 | B2 | T0 | 1 | 0.025 | 1 | 0 |
| 3 | B3 | T1 | 1 | 0.20 | 1 | 5 |
| 4 | B4 | T2a | 1 | 0.37 | 1 | 10 |
| 5 | | | | | | |
| 6 | | | | | | |
| 7 | | | | | | |
| 8 | B5 | T2b | 2 (2 sprays in 1 | 0.74 | 1 | 10 |
| 9 | | | day | | | |
| 10 | | | | | | |
| 11 | | | | | | |
| 12 | B6 | T2c | 6 (2 sprays | 2.2 | 1 | 10 |
| 13 | | | each | | | |
| 14 | | | day for 3 | | | |
| 15 | | | consecutive | | | |
| | | | days) | | | |
| 16 | B7 | T3 | 6 (2 sprays | 3.2 | 1 | 15 |
| 17 | | | each day | | | |
| 18 | | | for 3 | | | |
| 19 | | | consecutive | | | |
| | | | days) | | | |

TABLE 3.2
Dose of Formulations Tested for Nasal Mucosa Irritation Study.

| | | Bile | | | | | Dose | |
|---|---|---|---|---|---|---|---|---|
| | API | Salt | | | | | of | Num- |
| No. | (Epi) | Conc. | Mean | | | | Bile | ber |
| (From | Conc. | (STC) | Body | | | | Salt | of |
| Table | (mg/ | (mg/ | Weight | Dose of Epi | | | (STC) | rats |
| 4) | mL) | mL) | (kg) | d, mg | mg/kg | MRDH | (kg/mg) | (n) |
| 1 | 0 | 0 | 0.24 | 0 | 0 | 0 | 0 | 20 |
| 2 | 1 | 0 | 0.26 | 0.025 | 0.10 | 10 | 0.0 | 20 |
| 3 | 1 | 5 | 0.27 | 0.025 | 0.09 | 9 | 0.5 | 20 |
| 4 | 1 | 10 | 0.31 | 0.025 | 0.08 | 8 | 0.8 | 20 |
| 5 | | | 0.26 | 0.025 | 0.09 | 9 | 0.9 | 20 |
| 6 | | | 0.25 | 0.025 | 0.10 | 10 | 1.0 | 20 |
| 7 | | | 0.22 | 0.025 | 0.11 | 11 | 1.1 | 20 |
| 8 | 1 | 10 | 0.27 | 0.05 | 0.19 | 19 | 1.9 | 20 |
| 9 | | | 0.27 | 0.05 | 0.19 | 19 | 1.9 | 20 |
| 10 | | | 0.25 | 0.05 | 0.20 | 20 | 2.0 | 20 |
| 11 | | | 0.23 | 0.05 | 0.22 | 22 | 2.2 | 20 |
| 12 | 1 | 10 | 0.30 | 0.15 | 0.50 | 50 | 5.0 | 20 |
| 13 | | | 0.27 | 0.15 | 0.55 | 55 | 5.5 | 20 |
| 14 | | | 0.25 | 0.15 | 0.59 | 59 | 5.9 | 20 |
| 15 | | | 0.23 | 0.15 | 0.65 | 65 | 6.5 | 19 |
| 16 | 1 | 15 | 0.30 | 0.15 | 0.50 | 50 | 7.5 | 20 |
| 17 | | | 0.28 | 0.15 | 0.55 | 55 | 8.2 | 20 |
| 18 | | | 0.26 | 0.15 | 0.58 | 58 | 8.7 | 20 |
| 19 | | | 0.23 | 0.15 | 0.65 | 65 | 9.8 | 19 |

Because this study is for assessment of nasal mucosa irritation by bile salt (STC), the concentration of the API (epinephrine) was fixed at 1 mg/mL. The IN delivery volume for each spray is 25 μL. The rats were treated by one (1) IN spray (Arms T1, T2a), two (2) IN sprays (Arm T2b), and six (6) IN sprays (Arms T2c and T3). The six-spray treatments in Arms T2c and T3 were conducted as two sprays a day for 3 days consecutively. These two types of treatments, Arms T2c and T3, with six (6) IN sprays (as shown in Table 4) were designed for the extreme test of the nasal irritation caused by bile salt (STC).

Also in Table 4, the quantity B represents the net API epinephrine absorption, which is defined as B=d×EF×K, where d is the dose of epinephrine (in mg), EF is the Bile Salt EF, and K is the number of sprays of the IN formulation. Also in Table 5, MRDH is the Maximum Relative Dose for Human for Epipen®, 0.01 mg/kg.

Also, for Example 3, the excipients (not shown in Table 4) in these formulations include about 4 mg/mL citric acid (monohydrate), about 8 mg/mL sodium citrate (dihydrate), about 5.5 mg/mL chlorobutanol (hemihydrate), about 2-3 mg/mL sodium chloride, about 0.02 mg/mL EDTA (dihydrate), about 0.3 mg/mL sodium metabisulfite, and water for injection (q.s.). In addition, hydrochloric acid and sodium hydroxide may be added, as needed, to adjust the pH of the formulations to about 3.8.

The histopathology of nasal tissue was examined to evaluate nasal mucosa tolerance to each test articles with respect to bile salt (STC) amounts. In order to study the damage recoverability, the rat histopathological studies were conducted at 4 hours, 3 days, 1 week, and 2 weeks after the last treatments for Arms-T2a, T2b, T2c, and T3, as summarized in Table 3.3.

TABLE 3.3
Results of Nasal Mucosa Irritation Study Using Bile Salt As Absorption Enhancer for
IN Delivery.

| | | Bile Salt | | | | | | Average Irritation | | |
|---|---|---|---|---|---|---|---|---|---|---|
| No. (From | API (Epi) Conc. | Conc. (STC) | Total Number of Findings | | | | | Per Rat ** | | |
| Table 4) | (mg/mL) | (mg/mL) | m_{g=1} | m_{g=2} | m_{g=3} | m_{g=4} | TIP* | M^{1,2} | M^{3,4} | P |
| 1 | 0 | 0 | 12 | 5 | 0 | 0 | 22 | 0.9 | 0.0 | 1.1 |
| 2 | 1 | 0 | 6 | 0 | 0 | 0 | 6 | 0.3 | 0.0 | 0.3 |
| 3 | 1 | 5 | 13 | 0 | 0 | 0 | 13 | 0.7 | 0.0 | 0.7 |
| 4 | 1 | 10 | 14 | 11 | 9 | 2 | 71 | 1.3 | 0.6 | 3.6 |
| 5 | | | 18 | 1 | 0 | 0 | 20 | 1.0 | 0.0 | 1.0 |
| 6 | | | 3 | 0 | 0 | 0 | 3 | 0.2 | 0.0 | 0.2 |
| 7 | | | 7 | 0 | 0 | 0 | 7 | 0.4 | 0.0 | 0.4 |
| 8 | 1 | 10 | 37 | 16 | 8 | 2 | 101 | 2.7 | 0.5 | 5.1 |
| 9 | | | 32 | 5 | 0 | 0 | 42 | 1.9 | 0.0 | 2.1 |
| 10 | | | 8 | 1 | 0 | 0 | 10 | 0.5 | 0.0 | 0.5 |
| 11 | | | 10 | 0 | 0 | 0 | 10 | 0.5 | 0.0 | 0.5 |
| 12 | 1 | 10 | 31 | 18 | 4 | 0 | 79 | 2.5 | 0.2 | 4.0 |
| 13 | | | 40 | 21 | 5 | 0 | 97 | 3.1 | 0.3 | 4.9 |
| 14 | | | 14 | 0 | 0 | 0 | 14 | 0.7 | 0.0 | 0.7 |
| 15 | | | 6 | 1 | 0 | 0 | 8 | 0.4 | 0.0 | 0.4 |
| 16 | 1 | 15 | 69 | 22 | 14 | 11 | 199 | 4.6 | 1.3 | 10.0 |
| 17 | | | 38 | 46 | 12 | 0 | 166 | 4.2 | 0.6 | 8.3 |
| 18 | | | 33 | 3 | 0 | 0 | 39 | 1.8 | 0.0 | 2.0 |
| 19 | | | 9 | 1 | 0 | 0 | 11 | 0.5 | 0.0 | 0.6 |

In Table 3.3, TIP is Total irritation points, defined as p=m₁+2 m₂+3 m₃+4 m₄, mj is the number of observations with Grade-j; M^{1,2}=the average of m₁+m₂; M3,4=average of m₃+m₄; P—average of TIP.

The histopathological evaluation for Example 3 was conducted by a qualified pathology laboratory. In total, 55 types of microscopic findings in Turbinate I to IV were assessed. The severity of the microscopic findings was reported as Grade 1 to 5 as follows:

Grade 1 Minimal.

Grade 2 Mild—a noticeable but not a prominent feature of the tissue.

Grade 3 Moderate—a prominent but not a dominant feature of the tissue.

Grade 4 Marked—a dominant but not an overwhelming feature of the tissue.

Grade 5 Severe—an overwhelming feature of the tissue.

No Grade 5 finding was reported for this nasal irritation study among all 378 samples examined for 20,790 (=55×378) evaluated histopathological items. The total number of findings with Grades 1, 2, 3, 4 (denoted as mg=1, mg=2, mg=3, and mg=4, respectively, note no g=5 finding) for each arm at each assessment time are summarized in Table 3.3.

The results show that a dose-related increase in the nasal cavity epithelial, inflammatory, and exudative changes was typically seen unilaterally after exposure to STC and Epi and was most evident at 4 hours in groups receiving ≥10 mg/mL STC with more than one administration, although Group B4 receiving 10 mg/mL STC was also affected fairly uniformly at a lesser severity than Groups B5-7 at 4 hours. Rapid repair from widespread erosion/flattening of respiratory epithelium was evident as respiratory epithelial cell hyperplasia with concomitant decreased cilia and less exudate and inflammation at three days. The repair progressed to sporadic findings at 1 and 2 weeks with many of the distal nasal cavities (Levels III and IV) from Groups B4-B7 being normal at 2 weeks post-dose.

### Quantitative Analysis of Data for Local Tolerance Histopathological Findings in Example 3.

In order to quantitatively analyze the data for histopathological findings the two (2) quantities, (i) total irritation point (TIP), denoted as p, and (ii) number of findings with Grades 3 and 4, denoted as m^{3,4}, among different treatment groups were analyzed. For a given rat belonging to a given arm and evaluation time, TIP and m^{3,4} are defined as follows:

p=m₁+2m₂+3m₃+4m₄ (Equation 1)

and

m^{3,4}=m₃+m₄ (Equation 2)

where m₁, m₂, m₃, m₄ are number of findings with Grade 1, 2, 3, 4, respectively, from the 55 microscopic histopathological items assessed. In the definition of TIP, higher grade has a greater weight, for example the weight for m₁ (number of Grade-1 findings) is 1 and the weight for m₄ (number of Grade-4 findings) is 4 in the definition of TIP as demonstrated in Equation 1.

TIP represents the global findings of local irritation caused by the bile salt (STC), and m^{3,4} reflects number of finding with higher grades. The average of TIP and m^{3,4}, are denoted as P and M^{3,4}, respectively, for a given arm and evaluation time:

$$P = \frac{1}{n} {\sum }_{j = 1}^{n} {p}_{j} \\ and \qquad ( Equation 3 )$$

$${M}^{3 , 4} = \frac{1}{n} {\sum }_{j = 1}^{n} {m}_{j}^{3 , 4} \qquad ( Equation 4 )$$

where n is the number of rats for the given treatment arm and given assessment time (4 hrs, 3 days, 1 week or 2 weeks). The quantitative data of P and M^{3,4} are provided in Table 3.3.

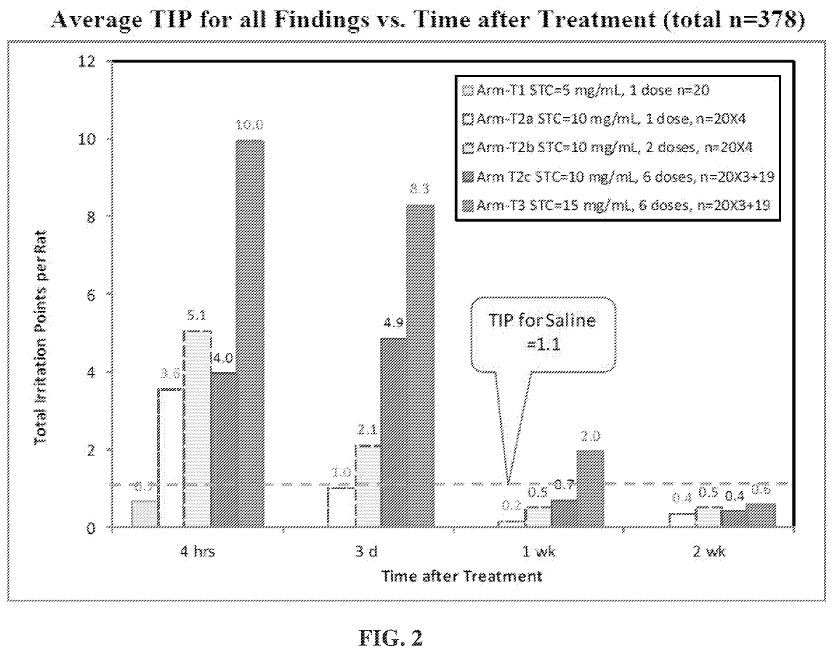

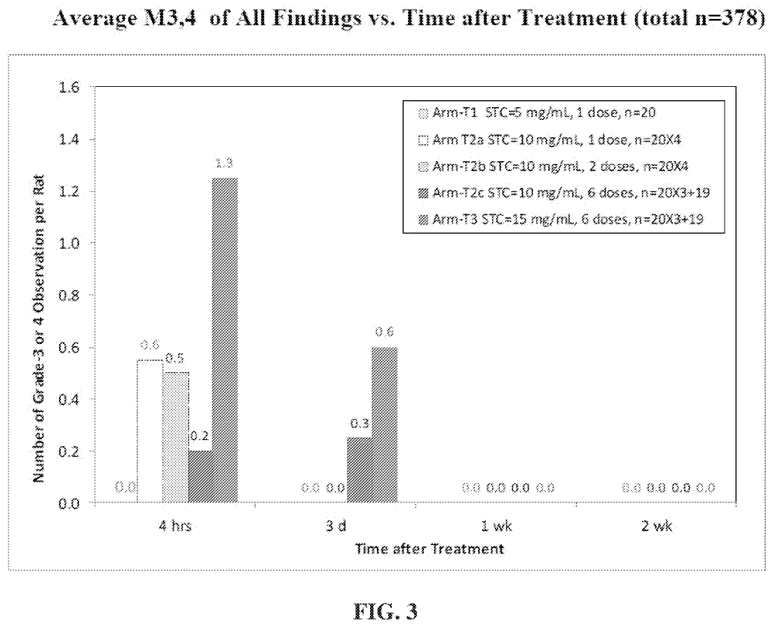

FIGS. 2-3 demonstrate the average TIP and M^{3,4}, respectively, for Arms-T1, T2a, T2b, T2c, and T3, at evaluation times of 4 hrs, 3 days, 1 week, and 2 weeks, respectively. Notably, the following profile is observed from FIGS. 2-3, which are consistent with the conclusions drawn in the study.

More irritation was observed for Arm-T3, 15 mg/mL;

For 1 or 2 IN sprays, 3 days after treatment, a quick reduction of the findings shows a “rapid repair”;

For 6 IN sprays (an extreme test) with STC=10 mg/mL, 1 week after treatment, the TIP reduced to even below the saline treatment;

For 6 IN sprays (an extreme test) with STC=15 mg/mL, 2 week after treatment, the TIP reduced to even below that of the saline treatment; and

One (1) week after treatment, the M^{3,4} for all treatments became zero.

It is also notable that the highest STC dose treated in this local irritation study is 9.8 mg/kg, which is approximately 3.9 times of the NOAEL of 2.5 mg/kg that was observed in the general toxicity study for STC using an IN delivery.

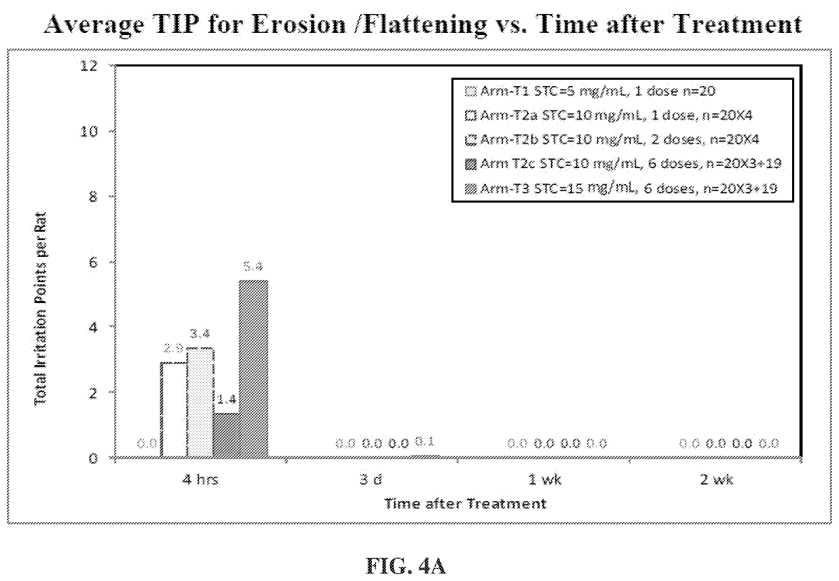

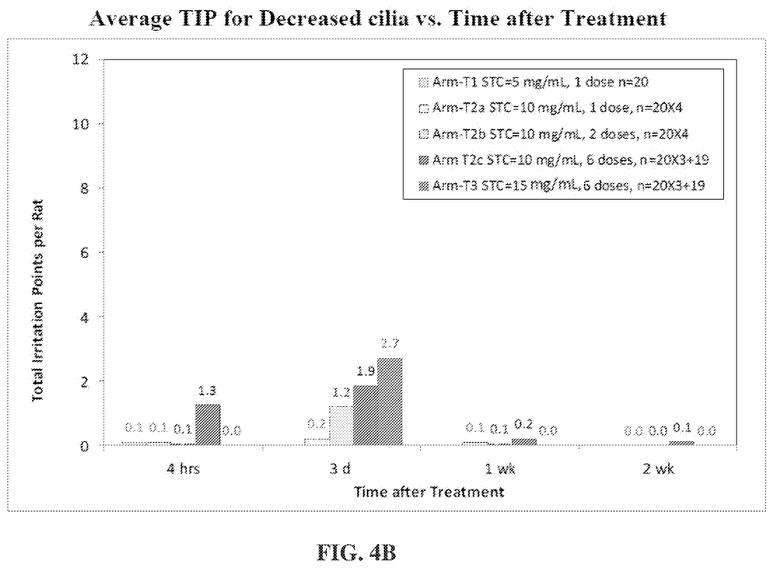

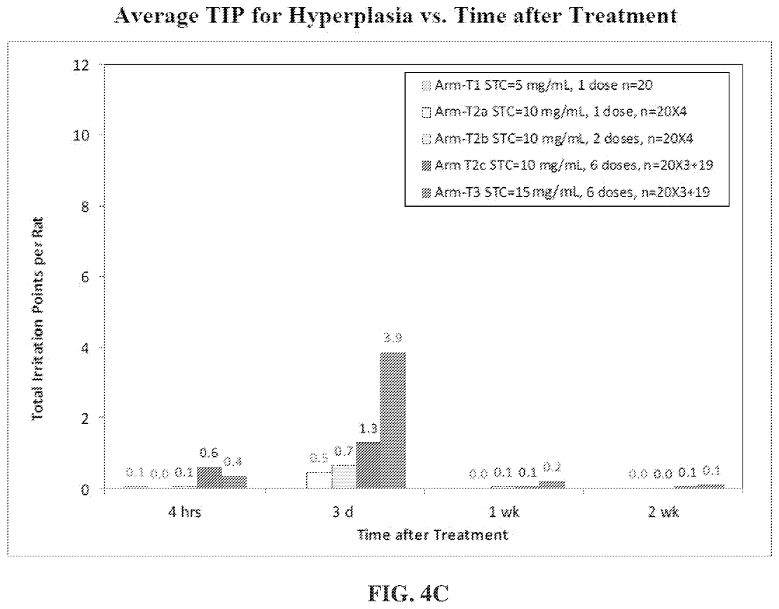

Among the 55 histopathological items assessed, the most frequent findings are (i) erosion/flattening, (ii) decreased cilia, and (iii) hyperplasia of respiratory epithelium. These 3 findings (the sum of observation in both turbinate I and II) are 63% of all findings based on the TIP assessment. FIGS. 4A-4B show the TIP for these three (3) types of findings.

FIG. 4A indicates that the TIP of erosion/flattening of respiratory epithelium reached the peak of the finding immediately (4 hrs after treatment), and can be rapidly repaired in 3 days, even for six (6) repeated sprays of the IN formulations at high STC concentration of 10 or 15 mg/mL (Arms T2c and T3).

FIGS. 4B-4C indicate that the TIP of the decreased cilia and hyperplasia, respectively of respiratory epithelium, both reached the peak of the findings on Day 3 after treatment, and can be repaired in 1 week, even for six (6) repeated sprays of the IN formulations at high STC concentration of 10 or 15 mg/mL (Arms T2c and T3). Thus, all damages/findings can be repaired or reversed in one (1) week to the negative control group level. Therefore, Example 3 demonstrates that the damages of “decreased cilia” are reversible based on this experimental study as demonstrated in FIG. 4B.

### Example 4—Bile Salts (STC) as Absorption Enhancer for IN Delivery of Naloxone

Example 4 is an animal study using sodium taurocholate (STC) as the bile salt for enhancing the absorption of a different API, namely naloxone. Example 4 was designed to study the relative bioavailability of naloxone in the route of intranasal administration (IN delivery) relative to administration by intramuscular injection (IM). The naloxone concentrations in rat's serum are determined at the baseline and at various post administration time points after IN delivery. The IN bioavailability is then calculated using PK data of area under the curve (AUC) and Cₘₐₓ.

Example 4 studies the effects of a bile salt, such as STC, on naloxone IN delivery using various formulations with STC concentrations ranging from 0 mg/mL to 8 mg/mL, and naloxone HCl of about 40 mg/mL. Table 4.1 provides the naloxone and STC formulations tested for Example 4. Note that the naloxone in the Table 4.1 formulations uses naloxone hydrochloride (HCl) dihydrate.

TABLE 4.1
Naloxone and STC Formulations Tested in Example 4 (Excipients not shown)

| | | API | | | | |
|---|---|---|---|---|---|---|
| | | (Naloxone) | | | | |
| | | Conc. | STC Conc. | Dose | Number | |
| Formulation | Delivery | (mg/mL)/ | (mg/mL)/ | Volume | of rats | |
| No. | Route | Dose (mg) | Dose (mg) | (μL) | (n=) | Description |
| 1 | IM | 40/1 | 0/0 | 25 | 8 | IM, N002-40-0 |
| 2 | IN | 40/1 | 0/0 | 25 | 8 | IN, N002-40-0 |
| 3 | IN | 40/1 | 6/0.15 | 25 | 8 | IN, N002-40-6 |
| 4 | IN | 40/1 | 8/0.20 | 25 | 8 | IN, N002-40-8 |

In Table 4.1, Formulations Nos. 1-2 also included about 2.75 mg/mL of sodium chloride as a tonicity agent, pH adjustor as needed (10% HCl), pH 4.2, and water (q.s.). Formulations Nos. 3-4 also included about 2.0 mg/mL of sodium chloride as a tonicity agent, pH adjustor as needed (10% HCl), pH 4.5, and water (q.s.).

For IN delivery of Formulations Nos. 2-4 of Table 4.1, each formulation was delivered by IN in the amount of 25 μL to the right nostril of the rat using a prefilled syringe. The rat was anesthetized to effect (isoflurane, 5% for approximately 5 minutes) before administration.

For the IM administration of Formulation No. 1 of Table 4.1, this formulation was intramuscularly injected in the amount of 25 μL to the right back thigh using a 31G insulin syringe (BD Insulin Syringe, 0.3 mL, ½ unit). To be consistent with the IN administration, the rats that received IM injection was also anesthetized (isoflurane, 5%) for 5 minutes before injection.

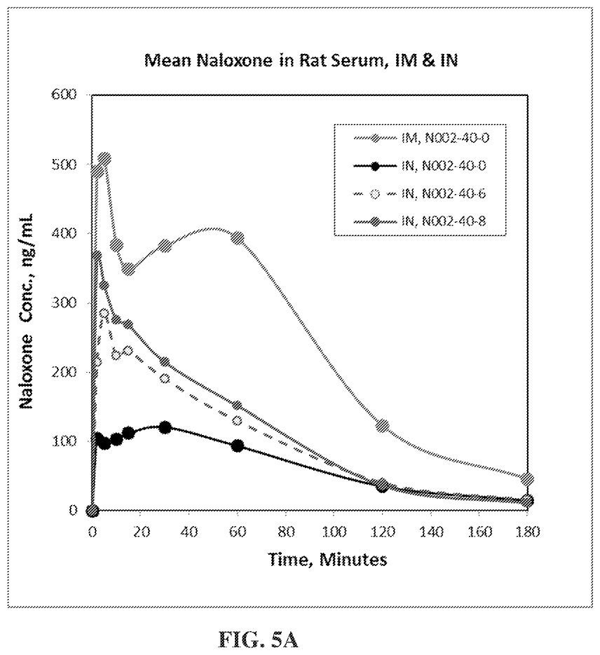

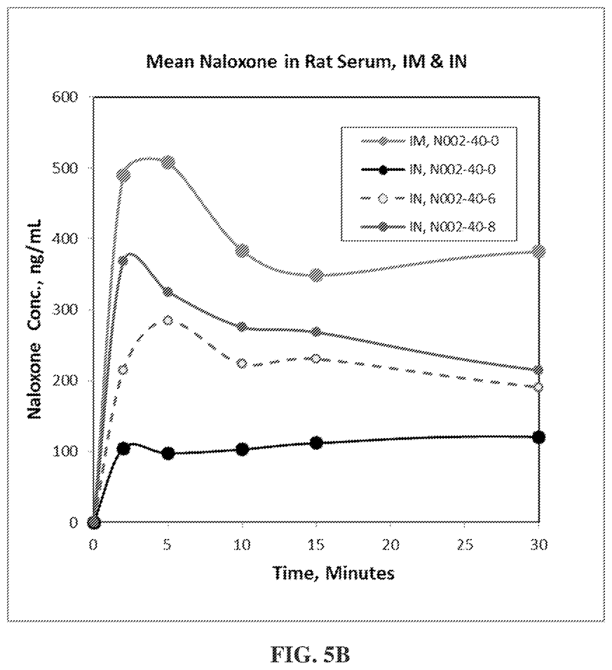

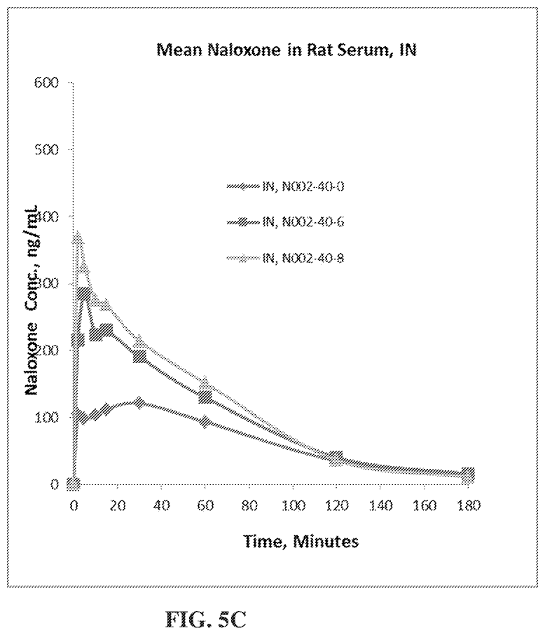

Tables 4.2-4.3 provide a summary of the PK results for Example 4. In Table 4.3, the IM Formulation No. 1 from Table 4.1 was used as the reference IM for determining the relative bioavailabilities compared to the IN Formulations Nos. 2-4. Additionally, FIGS. 5A-5C depict some of the key PK results provided in Tables 4.2-4.3. In particular, FIG. 5A depict the mean naloxone concentration in rat serum from 0 min. to 180 mins. FIG. 5B depict the mean naloxone concentration rat serum from 0 min. to 30 mins. FIG. 5C depict the mean naloxone concentration in rat serum by IN delivery.

TABLE 4.2
Summary of PK Results for Example 4.

| Formulation | | | AUC, ng/mL * min | | | API | API |
|---|---|---|---|---|---|---|---|
| No. (From | Cₘₐₓ | tₘₐₓ, | AUC_{0-30 min.} ± | AUC_{0-60 min.} ± | AUC_{0-180 min.} ± | Dose | Dose |
| Table 4.1) | (ng/ml) | min | S.D. | S.D. | S.D. | (mg/kg) | (mg/m²) |
| 1 | 667 | 18 | 11526 ± 4146 | 23163 ± 5980 | 43726 ± 8257 | 3.6 | 24 |
| 2 | 162 | 18 | 3192 ± 1008 | 6409 ± 1592 | 11786 ± 3320 | 3.5 | 24 |
| 3 | 310 | 8.8 | 6528 ± 1721 | 11335 ± 2884 | 18118 ± 4797 | 3.5 | 23 |
| 4 | 414 | 6.6 | 7891 ± 2974 | 13382 ± 4823 | 20434 ± 6781 | 3.4 | 23 |

TABLE 4.3
Relative Bioavailability (RBA), IN vs. IM for Example 4.

| Formulation | Relative Bioavailability (RBA), IN vs. IM | | | | |
|---|---|---|---|---|---|
| No. (From | | | | Mean | |
| Table 7) | AUC_{0-30 min.} | AUC_{0-60 min.} | AUC_{0-180 min.} | AUC_{(0-30 min., 0-60 min., 0-180 min.)} | Cₘₐₓ |
| 2 | 28% | 28% | 27% | 27% | 24% |
| 3 | 57% | 49% | 41% | 49% | 46% |
| 4 | 68% | 58% | 47% | 58% | 62% |

Notably, as shown in Table 4.3, the mean relative bioavailability (RBA) of naloxone with no STC (Formulation No. 2 in Table 4.1) in IN delivery in rats is 27% relative to the IM administration route (Formulation No. 1 in Table 4.1). If the IN naloxone formulation contains 6 mg/mL of STC (Formulation No. 3 in Table 4.1), then the mean RBA is significantly increased to 49%, a factor of 1.8 times increase, compared to naloxone with no STC (Formulation No. 2 in Table 4.1). Moreover, if the IN naloxone formulation contains 8 mg/mL of STC (Formulation No. 4 in Table 4.1), then the mean RBA is significantly increased to 58%, a factor of 2.1 times increase, compared to naloxone with no STC (Formulation No. 2 in Table 4.1).

Also notable, the RBA for Cₘₐₓ also demonstrate similar enhancement effects by the bile salt, STC. In particular, as shown in Table 4.3, in Formulation No. 2, which does not contain STC, the naloxone RBA Cₘₐₓ is only 24%. When the formulation contains 6 mg/mL of STC such as in Formulation No. 3, the RBA Cₘₐₓ is increased to 46%, a factor of 1.8 times increase. Further, the formulation contains 6 mg/mL of STC such as Formulation No. 4, the RBA Cₘₐₓ is 58%, a factor of 2.1 times increase compared to no STC formulation.

Also notable, in Table 4.2, the time of tₘₐₓ in IN administration is shorter when 6 mg/mL or 8 mg/mL STC is added, compared to no STC in the formulation.

### Example 5—Bile Salts as Absorption Enhancer for IN Delivery of Epinephrine

Example 5 is an animal study using epinephrine as the API, and sodium taurochenodeoxycholate (STCDC) (0-10 mg/mL) and sodium taurocholate (STC) (0-20 mg/mL) as the bile salts for enhancing the absorption of epinephrine. Also studied is the IM control of 1 mg/mL epinephrine. This study was designed to study the absorption enhancement effect of taurochenodeoxycholate (TCDC) and taurocholate (TC). Table 5.1 details the formulations tested for Example 5, and Tables 5.1 and 5.2 provide the PK results.

TABLE 5.1
Formulations Tested for Example 5 (Excipients Not Shown)

| | | Epi Conc. | Bile Salt Conc. | | | |
|---|---|---|---|---|---|---|
| | | (mg/mL)/ | (mg/mL)/ | | Dose | Number |
| Formulation | Delivery | Dose | Dose | | Volume | of rats |
| No. | Route | (mg) | (mg) | pH | (μL) | (n=) |
| 1 | IM | 1/0.025 | 0/0 | 3.6 | 25 | 6 |
| 2 | IN | 1/0.025 | 0/0 | 3.6 | 25 | 6 |
| 3 | IN | 1/0.025 | STC: 2/0.05 | 3.6 | 25 | 6 |
| 4 | IN | 1/0.025 | STC: 5/0.125 | 3.6 | 25 | 6 |
| 5 | IN | 1/0.025 | STC: 10/0.25 | 3.6 | 25 | 6 |
| 6 | IN | 0.5 | STC: 20/0.05 | 3.6 | 25 | 6 |
| | | 0.0125 | | | | |
| 7 | IN | 1/0.025 | 0/0 | 3.6 | 25 | 6 |
| 8 | IN | 1/0.025 | STCDC: 2/0.05 | 3.6 | 25 | 6 |
| 9 | IN | 1/0.025 | STCDC: 5/0.125 | 3.6 | 25 | 6 |
| 10 | IN | 1/0.025 | STCDC: 10/0.25 | 3.6 | 25 | 6 |

In Table 5.1, Formulations Nos. 1-10 had excipients of about 8.5 mg/mL sodium chloride, about 3.84 mg/mL citric acid, about 1.5 mg/mL sodium metabisulfite, and pH adjustors (HCl 10%, NaOH) as needed to adjust the pH to about 3.6.

For the IN delivery, test article was intranasally (IN) delivered in the amount of 25 μL to the right nostril of the rat using a prefilled syringe. The rat was anesthetized to effect (isoflurane, 5% for approximately 5 minutes) before administration and returned to its cage after the administration.

For the IM administration, test article was intramuscularly injected in the amount of 25 μL to the right back thigh of the rat using a 31G insulin syringe (BD Insulin Syringe, 0.3 mL, ½ unit). To be consistent with the IN administration, the rats that received IM injection was also anesthetized to effect (isoflurane, 5% for approximately 5 minutes) before injection and returned to their cage after the injection.

Subsequently, the plasma samples from the rats were drawn post-dose (IM and IN) at 0 minute, 5 minutes, 10 minutes, 15 minutes, 30 minutes, 60 minutes, 120 minutes, and 180 minutes. The drawn plasma samples were analyzed for the PK results, as shown below.

TABLE 5.2
Summary of PK Results for Example 5.

| Formulation | | | | | | | |
|---|---|---|---|---|---|---|---|
| No. (From | Cₘₐₓ | tₘₐₓ, | AUC, ng/mL * min | | | AUC_{0-30 min.}/ | Epi Dose |
| Table 7) | (ng/mL) | min | AUC_{0-10 min.} | AUC_{0-30 min.} | AUC_{0-60 min.} | mg | (mg/kg) |
| 1 | 7.6 | 9 | 39.9 | 133 | 235 | 5332 | 0.092 |
| 2 | 1.1 | 75 | 2 | 12 | 32 | 484 | 0.094 |
| 3 | 0.7 | 65 | 3 | 12 | 31 | 493 | 0.093 |
| 4 | 1.7 | 18 | 9 | 27 | 51 | 1085 | 0.097 |
| 5 | 7.7 | 5 | 48 | 91 | 127 | 3659 | 0.103 |
| 6 | 3.8 | 9 | 24 | 65 | 111 | 5180 | 0.052 |
| 7 | 0.1 | 23 | 0.1 | 1 | 3 | 47 | 0.077 |
| 8 | 1.9 | 10 | 12 | 35 | 61 | 1386 | 0.076 |
| 9 | 8.9 | 8 | 58 | 117 | 164 | 4672 | 0.077 |
| 10 | 7.4 | 9 | 48 | 124 | 190 | 4957 | 0.082 |

TABLE 5.3
Relative Bioavailability (RBA), IN vs. IM for Example 5.

| Formulation | | | | |
|---|---|---|---|---|
| No. (From | Relative Bioavailability (RBA), IN vs. IM | | | Mean RBA, |
| Table 7) | AUC_{0-30 min.} | AUC_{0-60 min.} | Cₘₐₓ | IN v. IM |
| 2 | 9% | 14% | 14% | 12% |
| 3 | 9% | 13% | 10% | 11% |
| 4 | 20% | 22% | 22% | 21% |
| 5 | 69% | 54% | 101% | 75% |
| 6 | 97% | 95% | 100% | 97% |
| 7 | 1% | 1% | 1% | 1% |
| 8 | 26% | 26% | 25% | 26% |
| 9 | 88% | 70% | 116% | 91% |
| 10 | 93% | 81% | 97% | 90% |

In Table 5.1, the Mean RBA is the average of the RBAs for AUC_{0-30 min}, AUC_{0-60 min}, and Cₘₐₓ. Notably, as demonstrated in Table 5.1, Formulation No. 6 provides the highest mean RBA at 97% with respect to Formulation No. 1 (IM), thereby demonstrating that IN delivery can deliver a similar amount of epinephrine as in the IM route. In addition, as shown in Table 5.1, Formulation No. 6 has a similar tₘₐₓ as Formulation No. 1 (IM).

Also notable, as shown in Table 5.1, Formulation Nos. 9 and 10 also have a similar mean RBA, at 91% and 90% respectively, compared to Formulation No. 1 (IM), thereby demonstrating that IN delivery using STCDC as a bile salt can deliver a similar amount of epinephrine as in the IM route. In addition, as shown in Table 5.1, Formulation Nos. 9 and 10 has a similar tₘₐₓ as Formulation No. 1 (IM).

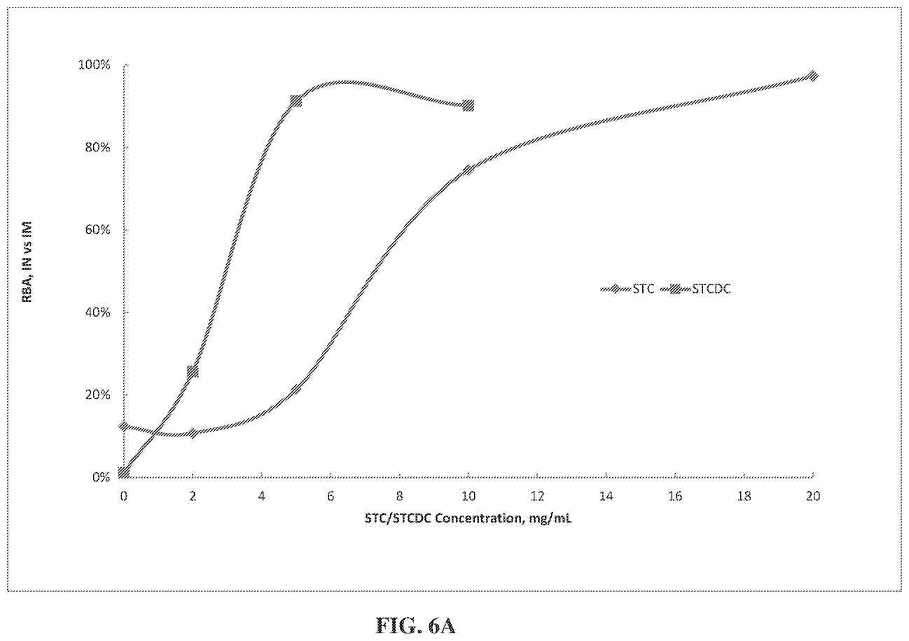

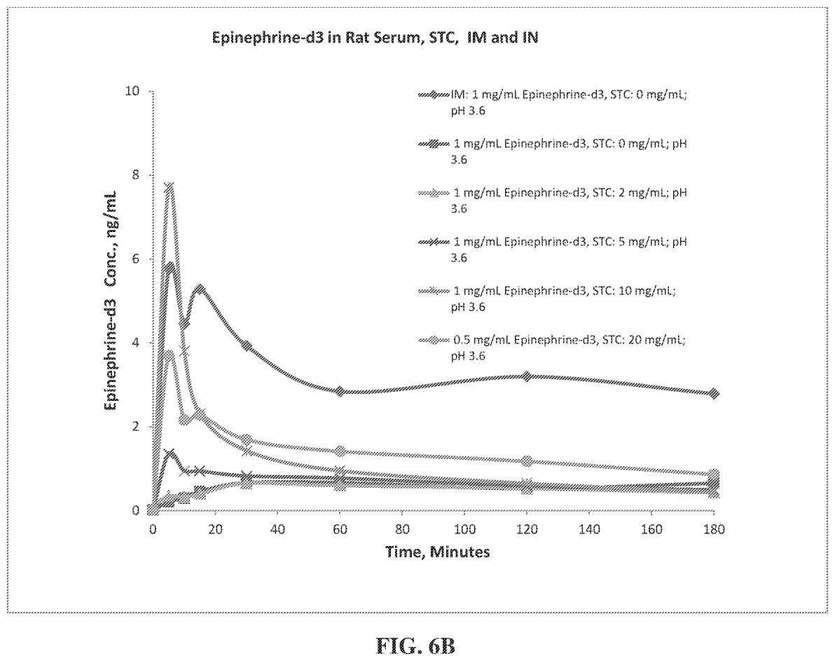

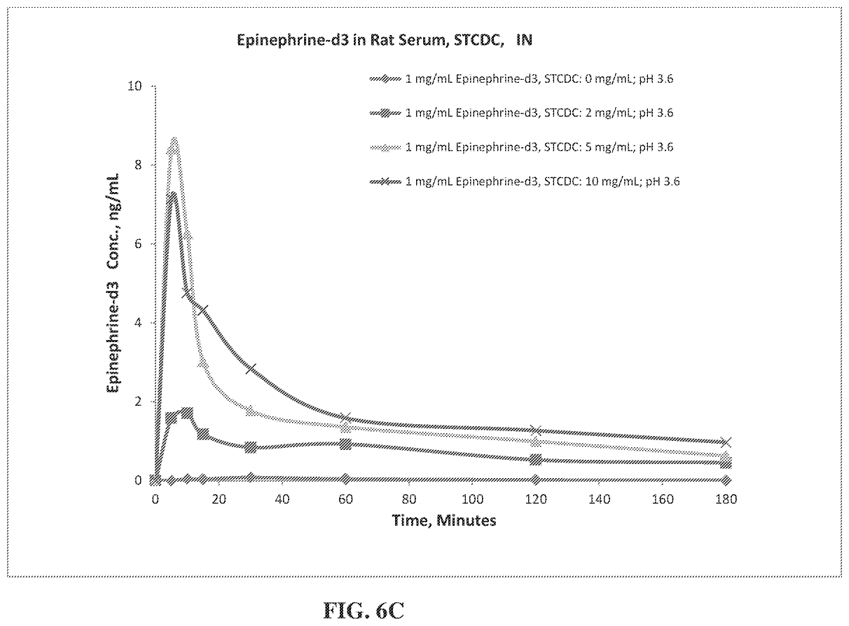

Additionally, FIGS. 6A-6C depict some of the key PK results shown in Tables 5.2 and 5.3. FIG. 6A is a graph illustrating the relative bioavailability of epinephrine, IN versus IM, in which the IN delivery uses either STC or STCDC. Notably, as shown in FIG. 6A, STCDC has better absorption enhancement effect than STC. For example, at a concentration of 5 mg/mL, the RBA was 91% for STCDC compared to STC's 21%.

FIG. 6B is a graph illustrating the mean epinephrine concentration in rat serum from 0 min to 180 mins utilizing STC as the bile salt. FIG. 6C is a graph illustrating the mean epinephrine concentration in rat serum from 0 min to 180 mins utilizing STCDC as the bile salt. Notably, as shown in FIGS. 6B-6C, when there is no bile salt in the formulation (see Formulation Nos. 2 and 7 of Table 5.1), the epinephrine bioavailability relative to IM administration (Formulation No. 1 of Table 5.1) was only 1-12%. Therefore, it can be concluded that adding a bile salt, such as STC or STCDC, to the formulation can enhance the epinephrine absorption into the bloodstream during IN delivery.

### Example 6—Local Toxicity Study of Bile Salt (STCDC) as Absorption Enhancer for IN Delivery of Epinephrine

Example 6 is an animal study designed to investigate the possible histological effects of bile salts (STCDC) on nasal mucosa of rats when the bile salt is used as an absorption enhancer for IN delivery. Table 6.1 shows the various IN formulations tested in Example 6, with STCDC as the representative bile salt absorption enhancer and epinephrine as the representative API. These IN formulations were administered intranasally to the rats. The histopathology of the rat's nasal tissue was examined to evaluate nasal mucosa tolerance to these tested IN formulations.

TABLE 6.1
Formulations Tested for Example 6 (Excipients Not Shown).

| | Epi Conc. | STCDC Conc. |
|---|---|---|
| | (mg/mL)/ | (mg/mL)/ |
| Group No. | Dose (mg) | Dose (mg) |
| 1 (Negative | 0/0 | 0/0 |
| Control) | | |
| 2 | 1/0.025 | 0/0 |
| 3 | 1/0.025 | 2/0.05 |
| 4 | 1/0.025 | 5/0.125 |
| 5 | 1/0.025 | 5/0.125 |
| 6 | 1/0.025 | 10/0.25 |

In Table 6.1, Group Nos. 2-6 each had excipients of about 8.5 mg/mL sodium chloride, about 3.84 mg/mL citric acid, about 1.5 mg/mL sodium metabisulfite, about 2.3 mg/mL HCl (10%), and a pH adjustor (NaOH) as needed to adjust the pH to 3.6. Group No. 1 is the negative control and is a saline nasal spray (CVS Health, Lot 6EK0606, Exp. 04/18) containing purified water, 0.65% of sodium chloride, disodium phosphate, phenylcarbinol, monosodium phosphate, and benzalkonium chloride as preservatives.

TABLE 6.2
IN Dose Treatment for Example 6.

| Group | | | | | | | |
|---|---|---|---|---|---|---|---|
| No. | | | | Sampling After Last Treatment | | | |
| (from | Number | | | (# of rats) | | | |
| Table | of rats | IN Dose | | 4 | 3 | 1 | 2 |
| 6.1) | (n=) | Frequency | IN Treatment | hrs | days | week | weeks |
| 1 | 8 | 2 × 25 μL | IN for 1 nostril | 8 | N/A | N/A | N/A |
| (Neg. | | | (25 μL), after | | | | |
| Cont.) | | | 15 seconds, | | | | |
| 2 | 8 | 2 × 25 μL | one more IN | 8 | N/A | N/A | N/A |
| 3 | 32 | 2 × 25 μL | to same nostril | 8 | 8 | 8 | 8 |
| 4 | 32 | 2 × 25 μL | (25 μL) | 8 | 8 | 8 | 8 |
| 5 | 32 | 2 × 25 | ibid, but for 3 | 8 | 8 | 8 | 8 |
| | | μL × 3 | consecutive | | | | |
| | | | days | | | | |
| 6 | 32 | 2 × 25 μL | ibid, for 1 day | 8 | 8 | 8 | 8 |

One hundred and forty four (144) rats (Male:Female=1:1) are randomly divided into six groups as listed in Table 6.2. Groups 1-2 each have four male and four female rats, while Groups 3-6 each had sixteen male and sixteen female rats. Each formulation is IN delivered in the amount of 25 μL to the right nostril using a prefilled syringe. The rat is anesthetized (isoflurane) before administration; remains under anesthesia for 3 minutes after administration and then returns to its cage. Fifteen (15) minutes after the first administration, the same test article is again intranasally delivered in the same amount of 25 μL to the same right nostril using the same procedures.

For rats in Group No. 5 of Table 6.2, the IN administrations are continued for the total of three consecutive days so that each rat receives six intranasal administrations of the Group No. 4. Rats in all other groups received only two intranasal administrations of the respective article.

Four (4) male and four (4) female rats were sacrificed by carbon dioxide at four time points (4 hr, 3 days, 1 week, and 2 weeks) after the last treatment, as specified in Table 6.2. The nasal passage/nasopharynx tissues was taken out. To help preserve the turbinate epithelium, formalin via injected into the nasopharyngeal opening until it came out of nares, then the whole tissue was immersed in 10% neutral buffered formalin. The tissue samples were sent to a qualified pathology laboratory for histopathologic evaluation.

In total, 48 types of microscopic findings in nasal turbinate cavity level I through IV were assessed, as detailed in Table 6.3.

TABLE 6.3
Summary of 48 Histopathologic Observations

| Nasal Cavity | Item of Histopathologic Observation | |
|---|---|---|
| Nasal Cavity I | 1. | Decreased; Cilia; Respiratory Epithelium |
| | 2. | Erosion/Flattening; Olfactory Epithelium |
| | 3. | Erosion/Flattening; Respiratory Epithelium |
| | 4. | Exudate; Serocellular; Meatus |
| | 5. | Hemorrhage; Meatus |
| | 6. | Hyperplasia; Respiratory Epithelium |
| | 7. | Increased Mucous Cells; Respiratory Epithelium |
| | 8. | Infiltrate; Neutrophils; Septum/Turbinates |
| | 9. | Necrosis; Individual Cell; Olfactory |
| Nasal Cavity II | 1. | Decreased; Cilia; Respiratory Epithelium |
| | 2. | Degeneration; Olfactory Epithelium |
| | 3. | Erosion/Flattening; Olfactory Epithelium |
| | 4. | Erosion/Flattening; Respiratory Epithelium |
| | 5. | Exudate; Serocellular; Meatus |
| | 6. | Foreign Material; Meatus |
| | 7. | Hemorrhage; Meatus |
| | 8. | Hyperplasia; Respiratory Epithelium |
| | 9. | Increased Mucous Cells; Respiratory Epithelium |
| | 10. | Infiltrate; Neutrophils; Septum/Turbinates |
| | 11. | Necrosis; Individual Cell; Olfactory Epithelium |
| Nasal Cavity III | 1. | Decreased; Cilla; Respiratory Epithelium; |
| | | Nasopharynx |
| | 2. | Degeneration; Olfactory Epithelium |
| | 3. | Diverticulum; Respiratory Epithelium; Septum |
| | 4. | Erosion/Flattening; Epithelium; Nasopharynx |
| | 5. | Erosion/Flattening; Olfactory Epithelium |
| | 6. | Erosion/Flattening; Respiratory Epithelium |
| | 7. | Exudate; Serocellular; Meatus |
| | 8. | Exudate; Serocellular; Nasopharynx |
| | 9. | Hemorrhage; Meatus |
| | 10. | Hyperplasia; Epithelium; Nasopharynx |
| | 11. | Hyperplasia; Respiratory Epithelium |
| | 12. | Infiltrate; Mixed Cell; Lateral Wall/Turbinate |
| | 13. | Infiltrate; Neutrophils; Nasopharynx |
| | 14. | Infiltrate; Neutrophils; Septum/Turbinates |
| | 15. | Necrosis; Individual Cell; Olfactory Epithelium |
| Nasal Cavity IV | 1. | Decreased; Cilla; Respiratory Epithelium; |
| | | Nasopharynx |
| | 2. | Degeneration; Olfactory Epithelium |
| | 3. | Erosion/Flattening; Epithelium; Nasopharynx |
| | 4. | Erosion/Flattening; Olfactory Epithelium |
| | 5. | Erosion/Flattening; Respiratory Epithelium |
| | 6. | Exudate; Serocellular; Meatus |
| | 7. | Exudate; Serocellular; Nasopharynx |
| | 8. | Hemorrhage; Meatus |
| | 9. | Hyperplasia; Epithelium; Nasopharynx |
| | 10. | Hyperplasia; Respiratory Epithelium |
| | 11. | Infiltrate; Neutrophils; Nasopharynx |
| | 12. | Infiltrate; Neutrophils; Septum/Turbinates |
| | 13. | Necrosis; Individual Cell; Olfactory Epithelium |

The severity of the microscopic findings was reported as Level 1 to 5 as follows: Level 1 (L-1) Minimal—inconspicuous to barely noticeable but so minor, small, or infrequent.

Level 2 (L-2) Mild—a noticeable but not a prominent feature of the tissue.

Level 3 (L-3) Moderate—a prominent but not a dominant feature of the tissue.

Level 4 (L-4) Marked—a dominant but not an overwhelming feature of the tissue.

Level 5 (L-5) Severe—an overwhelming feature of the tissue.

In order to quantitatively analyze the data for histopathological findings, the three (3) quantities, (i) total observation point (TOP), denoted as TOP, (ii) Average TOP per item per rat, and (iii) average occurrence for Level i (AOL)-i (i=1-5) were analyzed.

TOP=m₁+2m₂+3m₃+4m₄+5m₅ (1)

Average TOP=TOP/48/n (2)

AOL-i=mᵢ/48/n (3)

where m₁, m₂, m₃, m₄, and m₅ are number of findings with Level 1, 2, 3, 4, 5, respectively, from the 48 assessed microscopic histopathological items (as detailed in Table 6.3), n is the number of rats examined. In the definition of TOP, higher grade has a greater weight, for example the weight for m₁ (number of Level-1 findings) is 1 and the weight for m₄ (number of Grade-4 findings) is 4 in the definition of TOP as demonstrated in Eq. (1). Results of Average TOP and AOL for Level 3 (L-3), Level 4 (L-4), and Level 5 (L-5) are listed in Table 6.4.

TABLE 6.4
STCDC Toxicity Summary of L-3, L-4, and L-5 v. Time.

| | | Group | Group | Group | Group | Group | Group |
|---|---|---|---|---|---|---|---|
| Dose Groups | | 1 | 2 | 3 | 4 | 5 | 6 |
| Average | 4 hours | 3.4% | 2.6% | 28% | 45% | 63% | 66% |
| TOP per | 3 days | N/A | N/A | 9.4% | 30% | 52% | 55% |
| item per rat | 1 week | N/A | N/A | 3.1% | 5.7% | 11% | 16% |
| | 2 weeks | N/A | N/A | 0.5% | 0.3% | 0.5% | 2.6% |
| Average | 4 hours | 0.0% | 0.0% | 1.0% | 2.9% | 5.7% | 2.3% |
| occurrence | 3 days | N/A | N/A | 0.0% | 1.0% | 0.5% | 3.9% |
| for L-3 per | 1 week | N/A | N/A | 0.0% | 0.0% | 0.0% | 1.3% |
| item per rat, | 2 weeks | N/A | N/A | 0.0% | 0.0% | 0.0% | 0.0% |
| AOL-3 | | | | | | | |
| Average | 4 hours | 0.0% | 0.0% | 0.0% | 1.8% | 2.4% | 6.1% |
| occurrence | 3 days | N/A | N/A | 0.0% | 0.0% | 0.5% | 2.3% |
| for L-4 per | 1 week | N/A | N/A | 0.0% | 0.0% | 0.0% | 0.3% |
| item per rat, | 2 weeks | N/A | N/A | 0.0% | 0.0% | 0.0% | 0.0% |
| AOL-4 | | | | | | | |
| Average | 4 hours | 0.0% | 0.0% | 0.0% | 0.0% | 0.0% | 2.6% |
| occurrence | 3 days | N/A | N/A | 0.0% | 0.0% | 0.0% | 0.0% |
| for L-5 per | 1 week | N/A | N/A | 0.0% | 0.0% | 0.0% | 0.0% |
| item per rat, | 2 weeks | N/A | N/A | 0.0% | 0.0% | 0.0% | 0.0% |
| AOL-5 | | | | | | | |

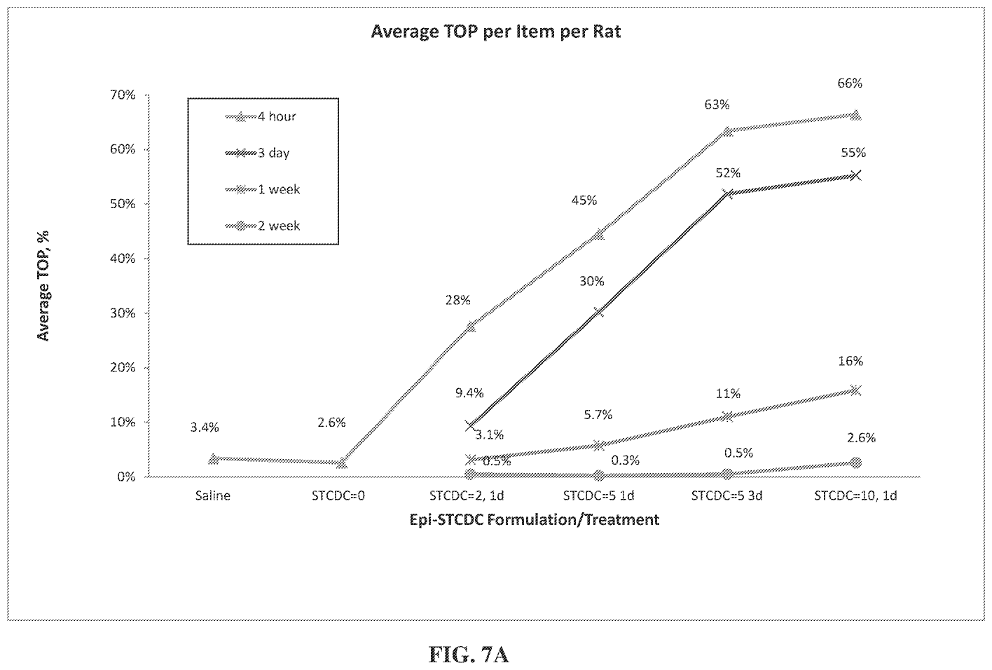

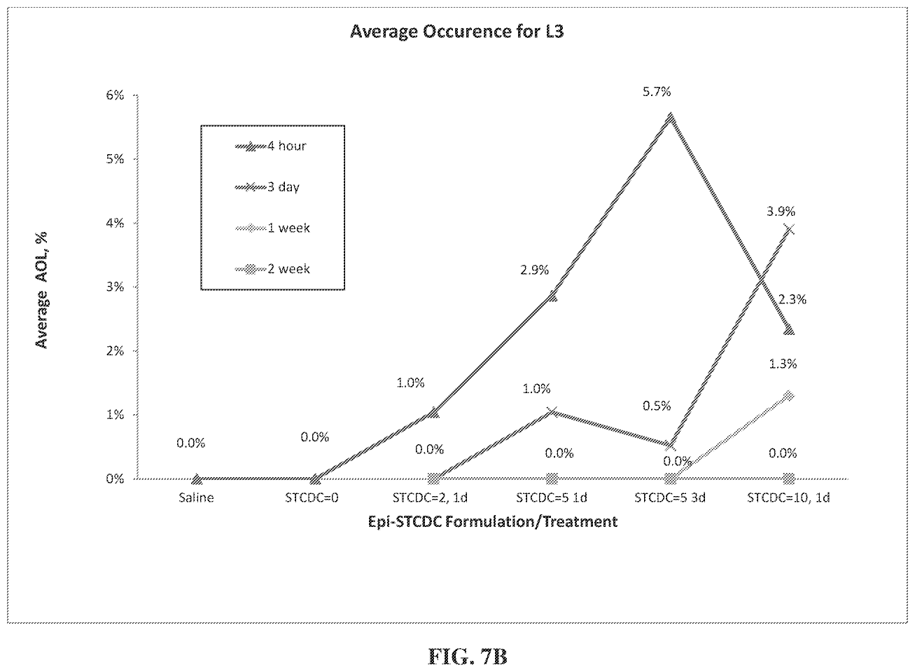

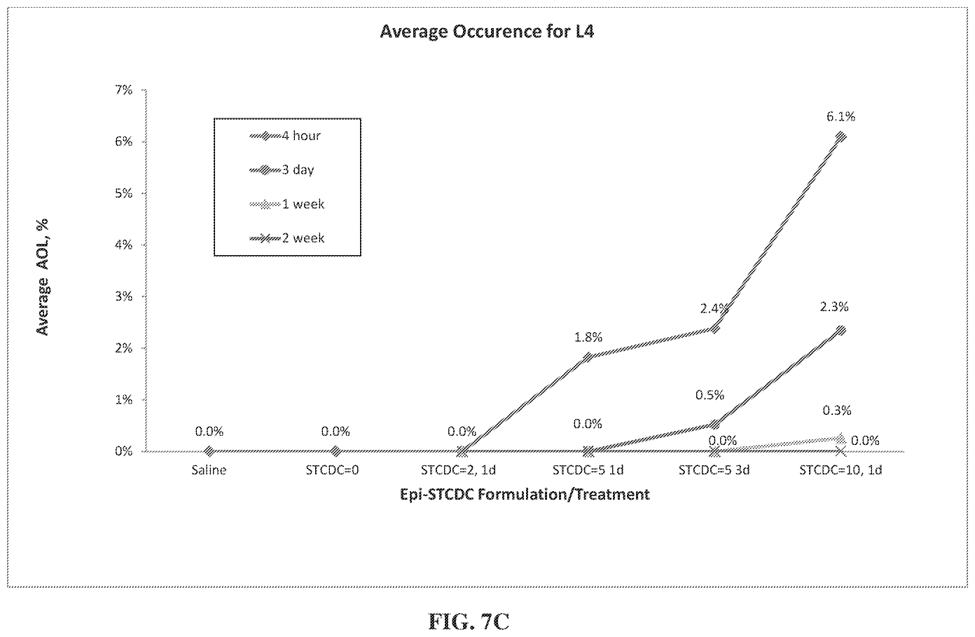

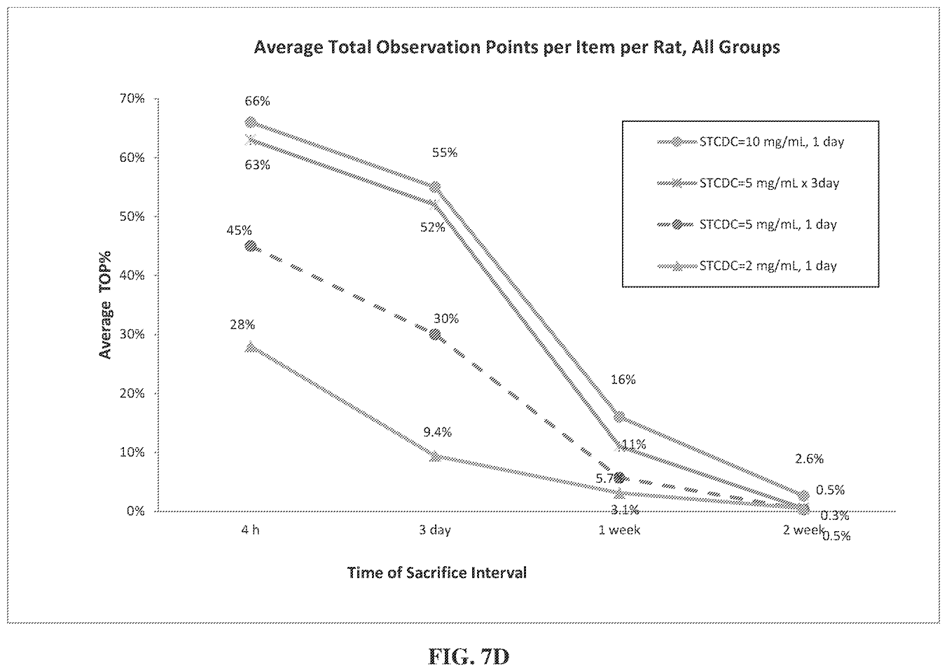

The STCDC toxicity summary results are detailed in Table 6.4 and FIGS. 7A-7C. As shown in these toxicity results, the toxicity of STCDC increases as its concentrations in the formulation increases. High doses of STCDC are associated with high percentages of average TOP. Within Groups 3-6, Group 3, which received the lowest dose of STCDC, had fewer changes than the higher doses of STCDC, particularly after four hours.

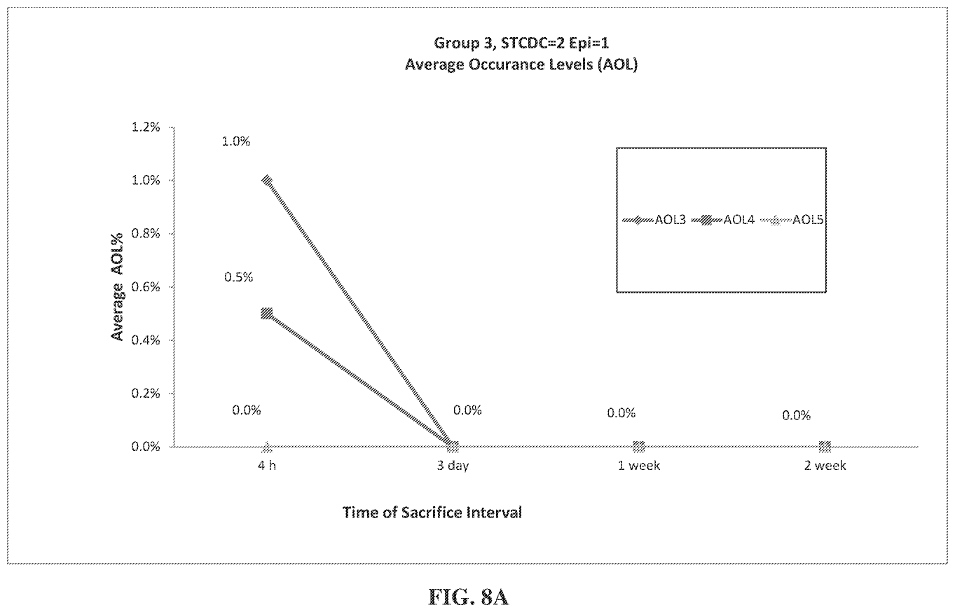

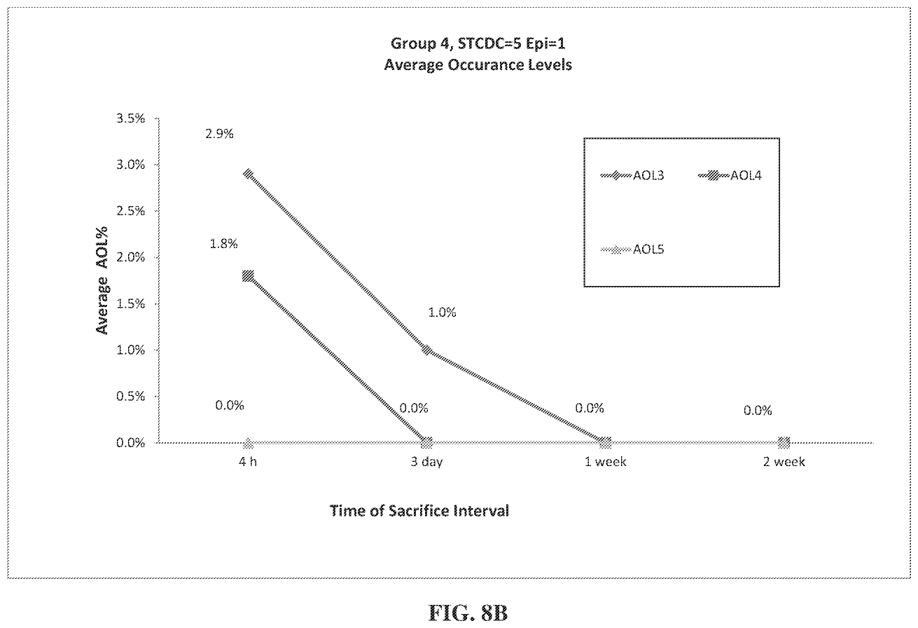

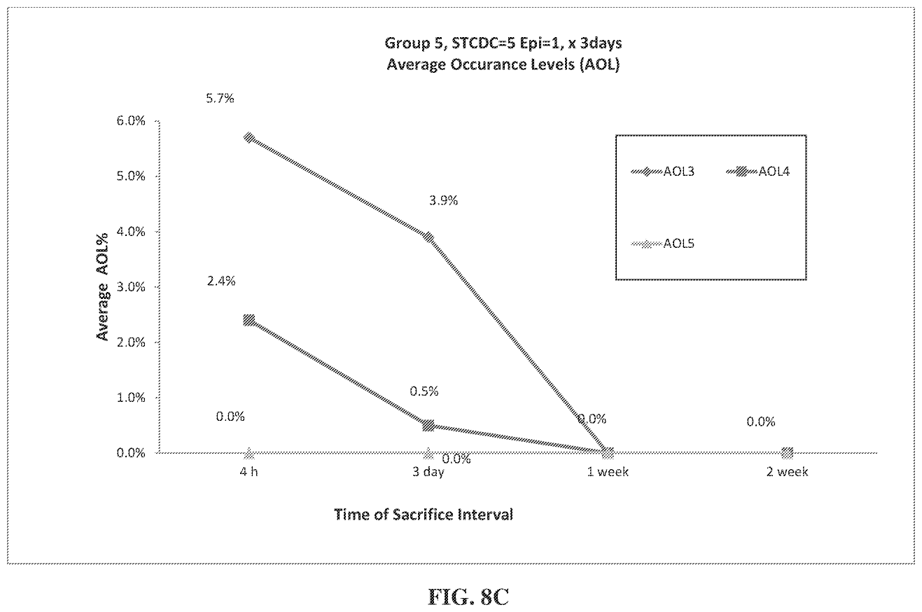

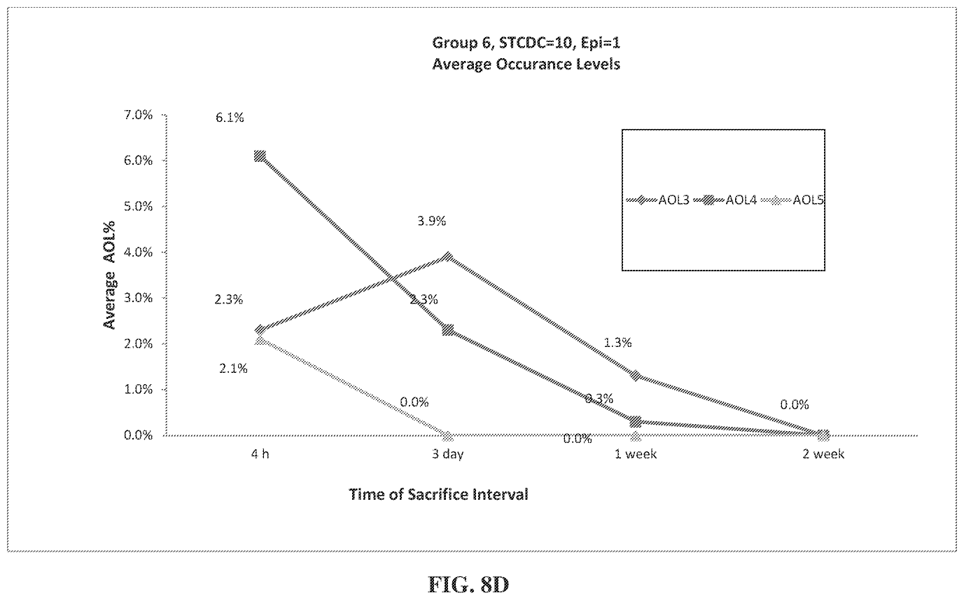

A dose-related increase in the nasal cavity epithelial, inflammatory, and exudative changes was observed after exposure to STCDC and epinephrine. When the average TOP is displayed versus time in FIG. 7D, most observed toxicity items occurred at 4 hours after administration, rapidly decreased over time, and nearly normal at two weeks post-dose. At about 1 week after administration, majority of the observed toxicity items disappeared. A similar decreasing over time trend can be observed for AOL-i (i=3,4,5) as shown in FIGS. 8A-8D. Thus, the average TOP percentages are decreasing rapidly from post administration times of 4 hours to two weeks. Therefore, this rapid decrease trend to nearly normal at two weeks post-done suggests that a bile salt, such as STCDC, is safe for clinical applications as an absorption enhancer.

### Example 7—Clinical Study Design with PK/PD Study Results for Intranasal Delivery of Epinephrine Using Sodium Taurocholate as an Enhancer and Formulations for Intranasal Delivery Using Sodium Taurocholate as an Enhancer

Example 7 presents a set of formulations with active ingredient epinephrine and bile salt Sodium Taurocholate (STC), one of primary bile salts existing in human blood, as the enhancer, intended for intranasal (IN) delivery.

TABLE 7.1
Formulations Intended for Intranasal Delivery

| Formulation (mg/mL) | IN1 | IN2 | IN3 | IN4 |
|---|---|---|---|---|
| L-Epinephrine | 12 | 12 | 6 | 6 |
| Sodium Taurocholate (STC) | 8 | 10 | 6 | 8 |
| Citric Acid Monohydrate | 4 | 4 | 4 | 4 |
| Sodium Citrate Dihydrate | 8 | 8 | 8 | 8 |
| Chlorobutanol Hemihydrate | 5.5 | 5.5 | 5.5 | 5.5 |
| EDTA Dihydrate | 0.02 | 0.02 | 0.02 | 0.02 |
| Sodium Chloride | 2 | 2 | 2 | 2 |
| Sodium Metabisulfite | 0.3 | 0.3 | 0.3 | 0.3 |
| NaOH/HCl | As needed for pH adjustment | | | |
| Water for Injection | QS Ad | QS Ad | QS Ad | QS Ad |
| pH | 3.8 | 3.8 | 3.8 | 3.8 |

### 7.1 Study Design

An open-label, active-controlled, single dose study was conducted to investigate the pharmacokinetics (PK), pharmacodynamics (PD), safety, and tolerability of the formulation described in Table 7.1 in two (2) cohorts of healthy volunteers.

The PK and PD parameters were compared between IN deliveries of the disclosed formulation and intramuscular (IM) injection of EpiPen® 0.3 mg/mL (Mylan; NDA 019430).

After satisfying the enrollment criteria, fifty-six (56) healthy volunteers were enrolled in two (2) sequential cohorts. Each cohort included twenty-eight (28) subjects who are investigated for IN dose and the active comparator by IM. Dosing in Cohort 2 started once all required subjects from Cohort 1 had completed their dosing sessions. The study treatments are listed in Table 7.2 below. The five (5) IN treatments with different API/STC doses are studied to investigate the effect of enhancer.

TABLE 7.2
Study Treatment for PK/PD Study

| Dose | IM | IN0 | IN1 | IN2 | IN3 | IN4 |
|---|---|---|---|---|---|---|
| L-Epinephrine (mg) | 0.3 | 1.2 | 1.2 | 1.2 | 0.6 | 0.6 |
| Sodium Taurocholate (STC) (mg) | 0 | 0 | 0.8 | 1 | 0.6 | 0.8 |
| Volume (mL) | 0.3 | 0.1 | 0.1 | 0.1 | 0.1 | 0.1 |
| Number of Subjects Treated | 28 | 28 | 24 | 24 | 27 | 27 |

### 7.2 PK Study and Results

Fifteen (15) blood samples were collected for each treatment of each subject at 0, 1′, 3′, 5′, 7′, 10′, 15′, 20′, 30′, 45′, 60′, 90′, 2 hrs, 4 hrs, and 6 hrs. Epinephrine was analyzed by a validated LC/MS/MS method with a quantitative limit of 10 pg/mL.

PK parameters for Cmax, AUCs, as geometric mean for each treatment are tabulated in Table 7.3 below.

TABLE 7.3
Major PK Results Summary

| PK Results, | | | | | | |
|---|---|---|---|---|---|---|
| geometric mean ± SD | IM | IN0 | IN1 | IN2 | IN3 | IN4 |
| Exogenous AUCo-t | 37.4 ± 2.8 | 3.0 ± 6.8 | 13.6 ± 10.7 | 32.5 ± 7.3 | 11.3 ± 4.4 | 15.2 ± 3.9 |
| (pg/mL*hr) | | | | | | |
| Cmax (pg/mL) | 457.7 ± 1.9 | 56.6 ± 2.0 | 330.0 ± 2.6 | 581.1 ± 3.0 | 77.2 ± 3.1 | 161.2 ± 2.7 |
| Tmax (min) | 11.1 ± 2.5 | 26.1 ± 5.1 | 10.8 ± 1.9 | 8.6 ± 2.4 | 17.6 ± 4.6 | 10.2 ± 2.4 |
| Relative | — | 1.9 ± 8.1 | 21.0 ± 2.7 | 34.6 ± 2.1 | 6.9 ± 6.3 | 22.7 ± 5.3 |
| Bioavailability, | | | | | | |
| RBA (%) | | | | | | |

Combined information in Table 7.2 and Table 7.3 demonstrated that

(1) In general, the added STC should reach to an appreciable level to play the role of an enhancer. The RBA of IN delivery increases from almost 0% without STC to 34.6% with STC.

(2) Intranasal delivery with STC could reach to a comparable level of drug absorption (Cmax, AUC and tmax) with that of standard IM route of administration.

(3) When a fast onset of effect is required, IN with STC might be more advantageous with respect to IM.

### 7.3 PD Study and Results

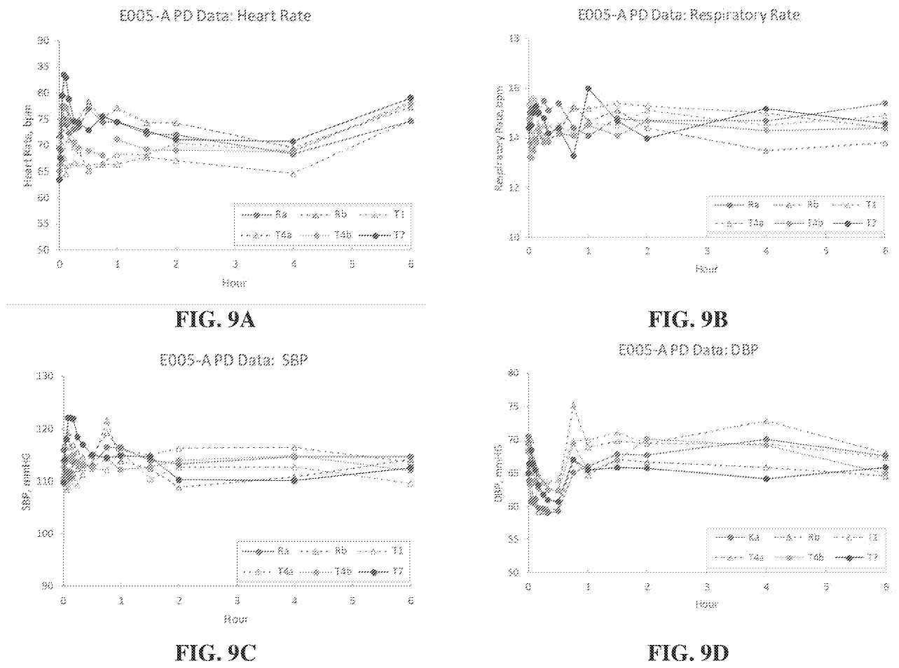

Vital signs (heart rate, systolic blood pressure, diastolic blood pressure, and respiratory rate) were measured at the same 15 time points as described in 2.2. FIG. 9A-9D show these vital signs heart rate, systolic blood pressure, diastolic blood pressure, and respiratory rate as a function of time for the six (6) treatments defined in Table 7.2.

FIGS. 9A-9D demonstrate that:
The PD data for every treatments, IM or IN, are comparable with excellent safety features;
The vital sign profile for treatments with STC and without STC does not show significant difference.

### Example 8—Safety and Local Tolerance Study Results for Intranasal Delivery of Epinephrine Using Sodium Taurocholate as an Enhancer

A total of 56 healthy subjects were studied as described in Section 7.1 above. The eight (8) IN treatments with different API/STC doses are studied to investigate the local tolerance of STC.

TABLE 8.1
Study Treatments for Safety Study

| | Ra | Rb | T1 | T6 | T7 | T3 | T4a | T4b | T2 | T5 |
|---|---|---|---|---|---|---|---|---|---|---|
| Dose | IM-1 | IM-2 | IN0 | IN1 | IN2 | IN3 | IN4 | IN4A | IN5 | IN6 |
| L-Epinephrine (mg) | 0.3 | 0.3 | 1.2 | 1.2 | 1.2 | 0.6 | 0.6 | 0.6 | 0.6 | 0.9 |
| Sodium Taurocholate | 0 | 0 | 0 | 0.8 | 1 | 0.6 | 0.8 | 0.8 | 0.4 | 0.8 |
| (STC) (mg) | | | | | | | | | | |
| Volume (mL) | 0.3 | 0.3 | 0.1 | 0.1 | 0.1 | 0.1 | 0.1 | 0.1 | 0.1 | 0.1 |
| Number of Subjects | 28 | 28 | 28 | 24 | 24 | 27 | 27 | 26 | 26 | 25 |
| Treated | | | | | | | | | | |

Local irritation was assessed by:

Nasal and Oropharyngeal Mucosa Examination (NOME);

Subjects Self-Reported Nasal Symptoms (SRNS); and

University of Pennsylvania Smell Identification Test (UPSIT).

Further, adverse drug events (ADEs) were also documented throughout the study period and analyzed.

### 8.1 Evaluation of Nasal and Oropharyngeal Mucosa Examination (NOME)

Nasal and Oropharyngeal Mucosa Examination (NOME) was used as the major assessment for local irritation. NOME was performed by an ENT specialist or qualified medical professionals. Compartments within nasal cavity and specific compartments within the oropharyngeal passage were evaluated, including the following seven (7) locations: (i) nasal floor, (ii) septum, and (iii) turbinates (iv) soft palate, (v) tonsil/tonsilar fossa, (vi) base of tongue, and (vii) the posterior pharyngeal wall. Locations (i) to (iii) are within the nasal cavity, and Locations (iv) to (vii) are within the oropharyngeal passage.

The examiner evaluated and recorded any abnormalities, including the following six (6) observations: (i) nasal irritation; (ii) mucosal erythema; (iii) mucosal edema; (iv) nasal discharge; (v) mucosal crusting; and (vi) mucosal epistaxis.

The nasal irritation was graded using the following scale:

| Grade | Description |
|---|---|
| 0 | no abnormal findings |
| 1A | focal nasal mucosal irritation (erythema, inflammation, or |
| | hyperemia) |
| 1B | superficial nasal mucosal erosion |
| 2 | moderate nasal mucosal erosion |
| 3 | nasal mucosal ulceration |
| 4 | nasal septum perforation |

If any mucosal irritation grade of >1B is noted during the course of the study, a comment describing the lesion(s) is required.

The other five (5) abnormalities were graded using the following scale: 0=none; 1=mild; 2=moderate; and 3=severe.

For each IN treatment, NOME was examined at baseline, 1 hour, and 6 hours post-dose. At the end of study for each cohort, the subject had a follow-up exam for NOME. In total, 4,857 NOME data points were examined.

Relative Change of Total NOME (RC-TNOME) vs. Amount of STC Used:

In order to evaluate the NOME response to the amount of STC used, the NOME data for the Treatments IN1, IN4 IN4A, IN6, with the same STC dose 0.8 mg, are combined together for evaluation. The relative change of total NOME (RC-TNOME) or baseline corrected total NOME versus STC dose at given time t and given set of NOME grades is defined as

F^{g}(d,t)=E^{g}(d,t)−E^{g}(d,0) (1)

Which is a function of STC dose d at given post-treatment time t for a given set of NOME grades. The 2^{nd} term E^{g}(d,0) is the same day baseline of NOME data. For d=0.8 mg, the average of the four treatments (IN1, IN4 IN4A, IN6) are used.

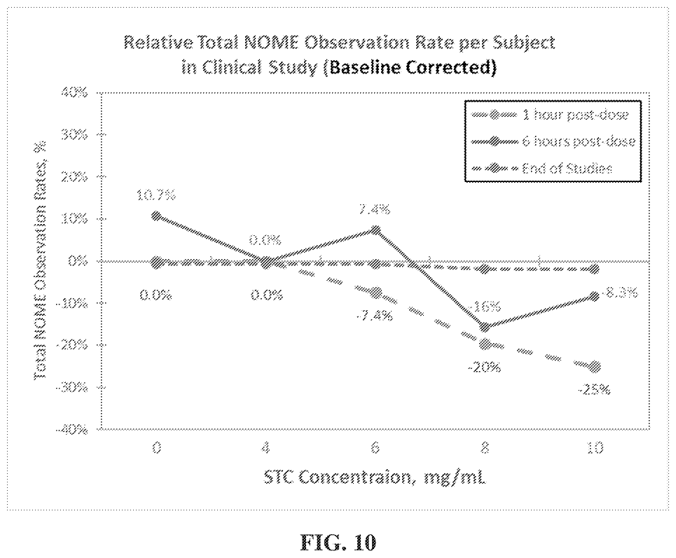

FIG. 10 provides plots for the RC-TNOME, F^{d,g} (t) defined by Eq. (1), where time is 1 and 6 hours post treatments (FIG. 10 provides Relative Changes of Total NOME versus STC Doses). Data in Table 8.2 and FIG. 10 demonstrate that

(1) At the end of study (about 2 weeks after treatment), the relative total NORM observation rate is close to 0%.

(2) At 6 hours post-treatment, the relative change of total NOME observations reduced comparing to that at 1 hour post-treatment.

(3) The relative change of total NOME versus the STC show a correlation.

### 8.2 Evaluation of Subjects Self-Reported Nasal Symptoms (SRNS)

For SRNS, all subjects were instructed to rate four (4) symptoms in the Total Nasal Symptom Score (TNSS): 1) rhinorrhea (runny nose); 2) nasal congestion; 3) nasal itching; and 4) sneezing. Given that the epinephrine formulation contains a nasal irritant STC, two (2) additional symptoms 5) nasal discomfort and 6) facial pain/pressure were also evaluated.

### 8.3 Evaluation of Smell Identification Test

University of Pennsylvania Smell Identification Test (UPSIT) is used for smell identification test. UPSIT is assessed as the following five (5) categories of olfactory functions (OF): (i) anosmia, (ii) severe microsmia, (iii) moderate microsmia, (iv) mild microsmia, and (v) normosmia.

### Observation for Local Irritation Caused by Epinephrine/STC

Based on the study and evaluation of NOME, SRNS and UPSIT with respect to nasal local irritation, the following observations were made:
Epinephrine/STC caused mild to moderate nasal irritation at turbinate or nasal discomfort in the STC concentration range of 6-10 mg/mL.
However, the rate for severe local irritation caused by Epinephrine/STC is very low.
No impact of Epinephrine/STC on smell function was observed.

### 8.4 Major ADEs Reported

The following six (6) ADEs or ADE groups are identified as the major ADEs in this clinical study:

(1) Tachypnea, 112 occurrences, 32.5% of all ADE;

(2) Cardiac Disorder (Bradycardia or Tachycardia), 77 occurrences, 22.3% of all ADEs;

(3) Vascular Disorder (Diastolic or systolic hypertension, hypotension), 50 occurrences, 14.5% of all ADEs;

(4) Nasal Oedema/Erosion, 41 occurrences, 11.9% of all ADEs;

(5) Other Nasal ADE (Nasal discomfort, Epistaxis, Nasal congestion, and Paranasal sinus discomfort), 21 occurrences, 6.1% of all ADEs; and

(6) Headache, 10 occurrences, 2.9% of all ADEs.

These six (6) major ADEs account for 90.1% of the reported ADEs.

The six (6) major ADEs are divided into 3 groups:

| Group | Definition |
|---|---|
| Group-1 | related to vital sign, including tachypnea, cardiac disorder and |
| | vascular disorder |
| Group-2 | related to IN Delivery including nasal oedema/erosion and other |
| | nasal ADE. |
| Group-3 | others, including headache |

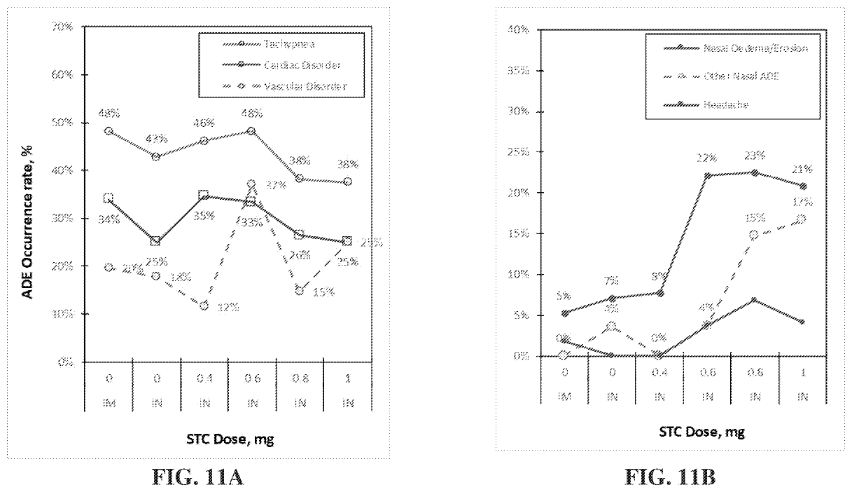

In order to assess the STC dose correlation with ADEs, the data for Treatments-IN1, IN4, IN4A, and IN6 were combined, and the data for Treatments-IM1 and IM2 are also combined. The ADE data for each individual major ADE vs. STC dose are summarized in Table 8.2 below. Plots of ADE occurrence rates vs. STC dose are given in FIG. 11A-11B.

FIG. 11A-B provide Rates of Major ADEs vs. STC Dose. FIG. 11A shows the curves of ADE rates vs. STC doses for Group-1 of the major ADEs; FIG. 11B is the curves of ADE rates vs. STC doses for Group-2 and 3 of the major ADEs (Left—Group-1, Vital Sign-related; Right Group-2 (IN-related) and Group-3 (Others)).

TABLE 8.2
Major ADEs and Their Occurrence Rates

| | | IM1, | | | | IN1, IN4, | |
|---|---|---|---|---|---|---|---|
| Items | Treatment | IM2 | IN0 | IN5 | IN3 | IN4A, IN6 | IN2 |
| | Delivery | IM | IN | IN | IN | IN | N |
| | STC, mg | 0 | 0 | 0.4 | 0.6 | 0.8 | 1 |
| | Epinephrine Dose, mg | 0.3 | 1.2 | 0.6 | 0.6 | 0.6-1.2 | 1.2 |
| | n= | 56 | 28 | 26 | 27 | 102 | 24 |
| Total ADE Occurrence | | 65 | 29 | 27 | 40 | 146 | 38 |
| ADE Rate, Occ. per Subject | | 116% | 104% | 104% | 148% | 143% | 158% |
| Major ADEs, Occurrences | | | | | | | |
| Group-1 | 1. Tachypnea | 27 | 12 | 12 | 13 | 39 | 9 |
| Vital Sign | 2. Cardiac disorders* | 19 | 7 | 9 | 9 | 27 | 6 |
| | 3. Vascular disorders** | 11 | 5 | 3 | 10 | 15 5 | 6 |
| Group-2 | 4. Nasal oedema/Erosion | 3 | 2 | 2 | 6 | 23 | 5 |
| IN-related | 5. Other Nasal ADE*** | 0 | 1 | 0 | 1 | 15 | 4 |
| Group-3 | 6. Headache | 1 | 0 | 0 | 1 | 7 | 1 |
| Others | | | | | | | |

| | | | | | | |
|---|---|---|---|---|---|---|
| Subtotal Occurrences | 61 | 27 | 26 | 40 | 126 | 31 |

*Bradycardia or Tachycardia.
**Diastolic or systolic hypertension, hypotension.
***Nasal discomfort, Epistaxis, Nasal congestion, and Paranasal sinus discomfort.

From Table 8.1 and FIGS. 11A and 11B, the following profile was observed:
For Group-1, the ADE rates for IN treatments at various doses of Epinephrine and STC are similar to that of IM treatment.
The ADE rates for Group-1 have no clear correlation with the STC doses.
For Group-2 and 3, the ADE rates for IN treatments at various doses of Epinephrine and STC are higher than that of IM treatment.
The ADE rates for Group-2 and 3 have a visible correlation with the STC doses, especially for Nasal Oedema/Erosion and Other Nasal ADE, etc.

### 8.5 Conclusion

Based on the study data for vital signs (PD), local irritation evaluations per NOME and SRNS, and ADE, one can see the following safety profile:
The impact of epinephrine formulation on the cardiovascular system and respiratory system are similar to that caused by the reference product (epinephrine by IM) based on PD and ADE profile.
Epinephrine/STC causes local irritations with the following profile:
   it causes certain rate of mild to moderate local irritations (nasal oedema, nasal discomfort) based on NOME, SRNS and ADE data;
   but the probability of severe local irritation is low per the data of NOME, SRNS and ADE;
   the reported local irritation is recoverable per the SRNS and ADE data. The irritations recovered in about 2 weeks.

### Example 9—Bile Salt (STC) as Effective Absorption Enhancer for IN Delivery of a Large Molecule API (Insulin Aspart)

Example 9 is an animal study in which a bile salt (STC) is used as an absorption enhancer for IN delivery of insulin aspart. This nonclinical study is designed to investigate the absorption enhancement effect of STC on Insulin Aspart in nasal mucosa in rats. Insulin aspart is indicated for improving glycemic control in adults and children with diabetes mellitus. Insulin aspart is homologous with normal human insulin with the exception of a single substitution of the amino acid proline by aspartic acid in position B28, and is produced by recombinant DNA technology. Insulin aspart has the empirical formula C₂₅₆H₃₈₁N₆₅O₇₉S₆ and a molecular weight of about 5825.8 g/mol. Thus, insulin aspart can be categorized as a large molecular API. This study is designed to study the effect of STC on Insulin Aspart nasal delivery using various insulin Aspart (I004) formulations with STC concentrations ranging from 0-15 mg/mL, and Insulin Aspart of 20 IU/mL. Results of plasma concentrations of Insulin Aspart at various time points are obtained. The STC absorption enhancement effect is evaluated using area under curve (AUC) results in subcutaneous (SC) and intranasal (IN) administrations. Table 9.1 provides the formulations tested in Example 9.

TABLE 9.1
Formulations Tested for Example 9 (Excipients Not Shown)

| | | | API Conc. | STC Conc. |
|---|---|---|---|---|
| Article No. | Article Name | Lot No. | (IU/mL) | (mg/mL) |
| Article 1 | I004-0 | I004-0-20IU | 20 | 0 |
| Article 2 | I004-5 | I004-5-20IU | 20 | 5 |
| Article 3 | I004-10 | I004-10-20IU | 20 | 10 |
| Article 4 | I004-15 | I004-15-20IU | 20 | 15 |

The treatment dose and treatment routes and other key information are listed in

TABLE 9.2
Study Design

| | | | | Insulin Aspart | Bile Salt | |
|---|---|---|---|---|---|---|
| Treatment | Article | | Dosing | Dose* | STC Conc. | Number of |
| # | Name | Delivery Route | Volume | mcg, mg/kg | mg/mL | rats (n=) |
| 1 | I004-0 | SC (Subcutaneous | 25 | 17.50/0.07 | 0 | 6 |
| | | injection) | | | | |
| 2 (T1) | I004-0 | IN | 25 | 17.50/0.07 | 0 | 6 |
| 3 (T2) | I004-5 | IN | 25 | 17.50/0.07 | 5 | 6 |
| 4 (T3) | I004-10 | IN | 25 | 17.50/0.07 | 10 | 6 |
| 5 (T4) | I004-15 | IN | 25 | 17.50/0.07 | 15 | 6 |

*Insulin aspart potency is 1U = 0.035 mg
Rat body weight: 0.25 Kg
Human dose: 0.1 U/Kg/Inj.
Rate Dose: 2 U/Kg (20× of human dose)

About 0.2 mL of whole blood was collected from the tail vein at pre-dose and at each specified post-dose time point, including at 0 min., 2 mins., 5 mins., 10 mins. 15 mins., 30 mins., 60 mins., 120 mins., and 180 mins. The relative bioavailability of Insulin Aspart in the route of intranasal administration is calculated using the Cₘₐₓ and AUC₀₋ₜ results in Table 9.3.

RBA=Parameter(IN)*Dose(SC)/Parameter(SC)*Dose(IN)

TABLE 9.3
Relative Bioavailability of Insulin Aspart in IN vs. SC Administration

| | Formulation | | | | | | Cₘₐₓ |
|---|---|---|---|---|---|---|---|
| | Insulin Aspart, | STC, | RBA vs. SC | | | | ratio vs. |
| Arm | IU/mL | mg/ml | AUC_{0-30′} | AUC_{0-60′} | AUC_{0-180′} | Mean | SC |
| | | | 2% | 2% | 4% | 0% | 0% |
| T1 | 20 | 0 | (noise) | (noise) | (noise) | | |
| T2 | 20 | 5 | 0% | 0% | 0% | 0% | 0% |
| T3 | 20 | 10 | 9% | 5% | 4% | 6% | 26% |
| T4 | 20 | 15 | 36% | 22% | 21% | 26% | 49% |

Tables 9.3-9.4 provide the PK results for Example 9 at 20 IU/mL. The only difference among these articles is the level of STC.

TABLE 9.4
Summary of PK Results for Example 9.

| | | | | | | | Insulin |
|---|---|---|---|---|---|---|---|
| Formu- | | | | | | | Aspart |
| lation | | | AUC, ng/mL * min | | | STC | Dosage |
| No. (From | Cₘₐₓ | tₘₐₓ, | AUC_{0-30 min.} ± | AUC_{0-60 min.} ± | AUC_{0-180 min.} ± | Dosage | (IU/kg)/ |
| Table 9.1) | (ng/mL) | min | S.D. | S.D. | S.D. | (mg/kg) | (mg/kg) |
| SC | 33 | 22 | 618 ± 211 | 1121 ± 396 | 1327 ± 416 | 0.0 | 1.848/52.8 |
| T1 | 1 | N/A | 14 ± 13 | 24 ± 22 | 58 ± 55 | 0.0 | 1.839/52.5 |
| T2 | 0 | N/A | 1 ± 2 | 1 ± 2 | 1 ± 2 | 0.4 | 1.762/50.3 |
| T3 | 8 | 4 | 56 ± 61 | 56 ± 61 | 56 ± 61 | 0.9 | 1.840/52.6 |
| T4 | 16 | 3 | 223 ± 160 | 242 ± 182 | 276 ± 188 | 1.3 | 1.720/49.1 |

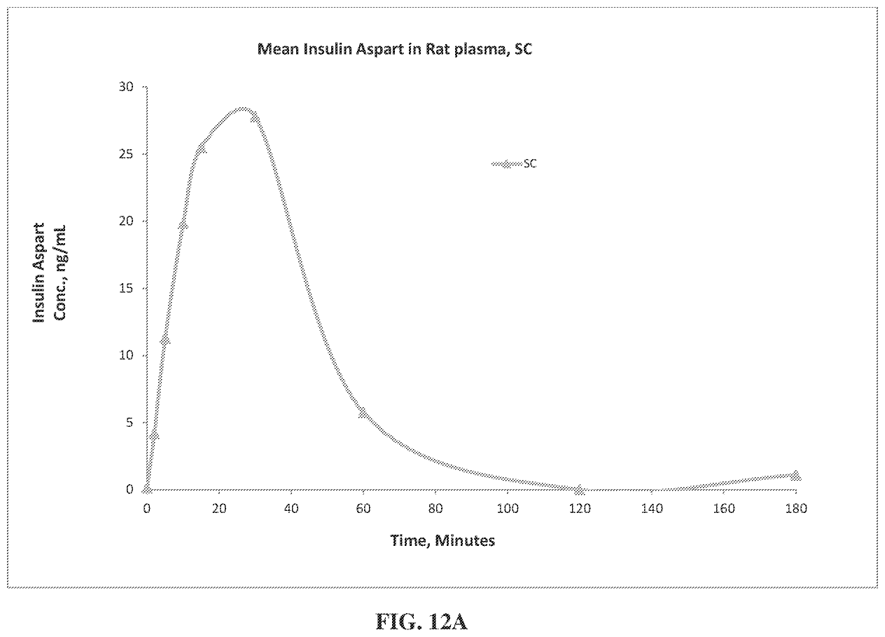

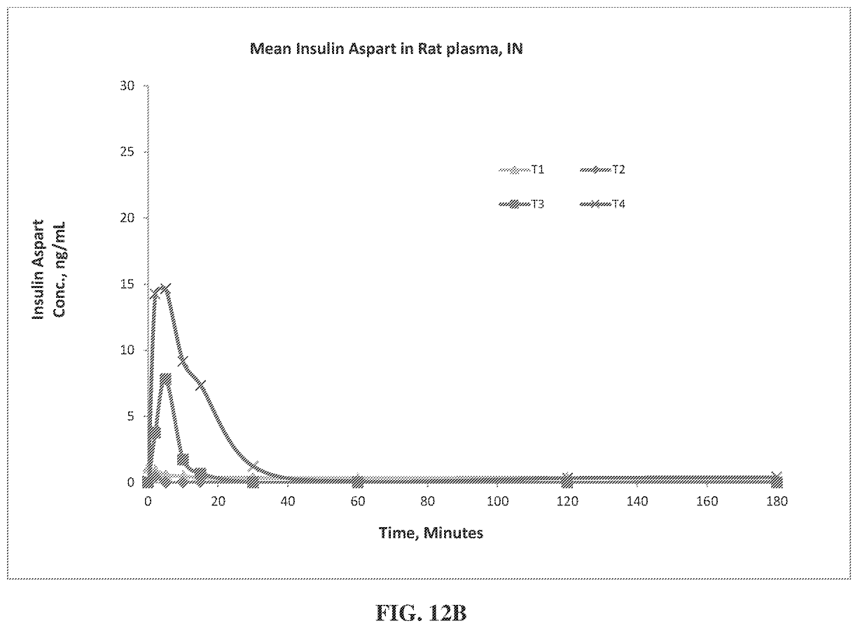

The PK results in Table 9.4 are also graphed in FIGS. 12A-12B. FIG. 12A is a graph illustrating the mean insulin aspart concentration in rat plasma from 0 min. to 180 mins. administered by SC injection. As can been seen from FIG. 12A, for the delivery route of SC injection, insulin aspart in plasma increased rapidly, reaching Cₘₐₓ in around 22 minutes, then decreased below detection limit in about two hours after injection.

FIG. 12B is a graph illustrating the mean insulin aspart concentration in rat plasma from 0 min. to 180 mins. for Arms T1-T4, which are delivered by IN administration. There were no insulin aspart detected in the rat's plasma in Arm T1 and Arm T2, which contained 0 and 5 mg/mL of STC in the formulation. When the STC concentration increased to 10 and 15 mg/mL in Arm T3 and Arm T4, insulin aspart can be detected, as shown in FIG. 9B, reaching Cₘₐₓ in about 3 to 4 minutes, then decreased to below detection limit in about one hour after IN administration.

Table 9.3 provides the relative bioavailability (RBA) of insulin aspart using IN administration compared to SC injection. The relative bioavailability of insulin aspart using IN administration is calculated using the Cₘₐₓ and AUC_{0-t*}

The Cₘₐₓ ratios are also provided in Table 9.3. Mean RBA were calculated using AUC of 0-30′, 0-60′ and 0-190′. Since the small numbers of AUC in Arm T1 were noise, the mean RBA was set to 0 for Arm T1. As can be seen, if the formation contains less than 5 mg/mL STC, the insulin aspart IN bioavailability relative to SC is 0 in Arms T1 and T2. When the formulation contains 10 mg/mL of STC, as in Arm T3, the RBA is increased to 6%; a small value but is detectable. In Arm T4 with 15 mg/mL of STC, the RBA is 26%, a factor of 4 times increase compared to 10 mg/mL STC formulation (Arm T3).

In conclusion, the relative bioavailability of Insulin Aspart in intranasal administration in rats is about 6% relative to the subcutaneous administration route if the STC is 10 mg/mL. The relative bioavailability will be increased to 26% if the STC is creased to 15 mg/mL in the formulation. On the other hand, there will be no bioavailability for insulin aspart if the STC concentration is below 5 mg/mL for the route of intranasal administration.

The Cₘₐₓ ratio (IN/SC) is 26% and 49%, respectively when STC is 10 and 15 mg/mL in the formulation. The time of tₘₐₓ in IN administration is shorter (3-4 min) when 10 or 15 mg/mL STC is added, compared to SC administration (22 min).

### Example 10—Local Tolerance Study and Reversibility of Histopathological Damages of Bile Salt STC as Absorption Enhancer for IN Delivery

This study was to performed to investigate the possible histological effects of Sodium Taurocholate (STC) on nasal mucosa in rats. Test formulations were prepared using various amounts of STC (an exemplary bile acid salt) and the active pharmaceutical ingredient (in this case, epinephrine). The formulations were intranasally administered into rats. The nasal tissue was histopathologically examined to evaluate mucosa tolerance to the test articles (e.g., the formulations) with respect to STC amount and multiple time points after the administration. The histopathological effects of STC on nasal mucosa and damage reversibility was investigated in 378 rats.

As noted above, test articles were prepared using various amounts of STC (0, 5, 10, and 15 mg/mL). Due to the fact that the study was for assessment of mucosa irritation by STC, the concentration of the API epinephrine was fixed at 1 mg/mL. The experiment design is summarized in Table 10.1.

TABLE 10.1
Study Design-Histopathological Local Tolerance Study-Epinephrine + STC.

| | | | | | | | Sampling after the | | | |
|---|---|---|---|---|---|---|---|---|---|---|
| | | | | | IN Treatment | | last treatment (# of rats) | | | |
| Group | Formulation | | | # of | # of | | 4 | 3 | 1 | 2 |
| # | Article# | Epi-d3 | STC | Rats | sprays | Description | hr | days | week | weeks |
| STC1 | Article 0 (saline) | 0 | 0 | 20 | 1 × 25 μL | IN for 1 nostril | 20 | | | |
| STC2 | Article 1 | 1 | 0 | 20 | 1 × 25 μL | | 20 | | | |
| STC3 | Article 2 | 1 | 5 | 20 | 1 × 25 μL | | 20 | | | |
| STC4 | Article 3 | 1 | 10 | 80 | 1 × 25 μL | | 20 | 20 | | 20 |
| STC5 | Article 3 | 1 | 10 | 80 | 2 × 25 μL | IN for 1 nostril, after | 20 | 20 | 2 | 20 |
| | | | | | | 15′ one more IN to | | | | |
| | | | | | | the same nostril | | | | |
| STC6 | Article 3 | 1 | 10 | 80 | 2 × 25 μL × 3 | ibid for 3 days | 20 | 20 | 20 | 20 |
| STC7 | Article 4 | 1 | 15 | 80 | 2 × 25 μL × 3 | | 20 | 20 | 20 | 20 |

| | | | | | |
|---|---|---|---|---|---|
| Total | 380 | 140 | 80 | 80 | 80 |

While the 4-hour post dose samples were used to assess instant local toxicity, samples from 3-day, 1-week and 2-week post-treatment were used to evaluate the reversibility, reparability of tissue damage.

Four levels of the nasal mucosa and turbinates were fixed in formalin, trimmed and processed to hematoxylin and eosin (H&E) slides. The four turbinate levels closely followed those as described through the histopathologic examination of the rat nasal cavity. In total, 55 types of microscopic findings in Turbinate I to IV were assessed. The severity of the microscopic findings was reported as Grade 1 to 5.

The study showed that “a dose-related increase in the nasal cavity epithelial, inflammatory, and exudative changes was typically seen unilaterally after exposure to STC and Epi-d3 and was most evident at 4 hours in groups receiving ≥10 mg/mL STC with more than one administration, although Group STC4 receiving 10 mg/mL STC was also affected fairly uniformly at a lesser severity than Groups STC5-STC7 at 4 hours. Rapid repair from widespread erosion/flattening of respiratory epithelium was evident as respiratory epithelial cell hyperplasia with concomitant decreased cilia and less exudate and inflammation at three days. The repair progressed to sporadic findings at 1 and 2 weeks with many of the distal nasal cavities (Levels III and IV) from Groups STC4-STC7 being normal at 2 weeks post-dose.” These results highlight the surprising and unexpected safety and tolerability of bile acids and salts thereof for the intranasal route.

### Example 11—Local Tolerance Study of Bile Salt STCDC as Absorption Enhancer for IN Delivery of Epinephrine

This study was to investigate the possible histopathological effects of Sodium Taurochenodeoxycholate (STCDC) on nasal mucosa in rats (n=144). Test articles were prepared in various amounts of STCDC (0, 2, 5, and 10 mg/mL) and active pharmaceutical ingredient (API) epinephrine (1 mg/mL) and intranasally administered into rats. The nasal tissue was histopathologically examined to evaluate mucosa tolerance to the test articles and damage reversibility with respect to STCDC amount and days after the administration. The experiment design is summarized in Table 10.

TABLE 11.1
Study Design-Histopathological Local Tolerance Study-Epinephrine + STCDC

| | | | | | | | Sampling after the | | | |
|---|---|---|---|---|---|---|---|---|---|---|
| | | | | | IN Treatment | | last treatment (# of rats) | | | |
| Group | Formulation | | | # of | # of | | 4 | 3 | 1 | 2 |
| # | Article# | Epi-d3 | STCDC | Rats | sprays | Description | hr | days | week | weeks |
| DC1 | Article 0 | 0 | 0 | 8 | 2 × 25 μL | IN for 1 | 8 | | | |
| | (saline) | | | | | nostril, after | | | | |
| DC2 | Article 4 | 1 | 0 | 8 | 2 × 25 μL | 15′ one | 8 | | | |
| DC3 | Article 5 | 1 | 2 | 32 | 2 × 25 μL | more IN to | 8 | 8 | 8 | 8 |
| DC4 | Article 6 | 1 | 5 | 32 | 2 × 25 μL | the same nostril | 8 | 8 | 8 | 8 |
| DC5 | Article 6 | 1 | 5 | 32 | 2 × 25 μL × 3 | ibid for 1 day | 8 | 8 | 8 | 8 |
| DC6 | Article 7 | 1 | 10 | 32 | 2 × 25 μL | ibid for 3 days | 8 | 8 | 8 | 8 |

| | | | | | |
|---|---|---|---|---|---|
| Total | 144 | 48 | 32 | 32 | 32 |

Four levels of the nasal mucosa and turbinates were fixed in formalin, trimmed and processed to hematoxylin and eosin (H&E) slides at a third party lab. The four turbinate levels closely followed those as described through the histopathologic examination of the rat nasal cavity. In total, 55 types of microscopic findings in Turbinate I to IV were assessed. The severity of the microscopic findings was reported as Grade 1 to 5.

The nasal tissue was histopathologically examined to evaluate mucosa tolerance to each test articles with respect to STCDC amounts. In order to study the damage recoverability, the rat histopathological studies were conducted at 4 hours, 3 days, 1 week, and 2 weeks after the last treatments.

The study results demonstrated no severe finding was reported for this nasal irritation study among all 144 samples examined for 7,920 (=55×144) evaluated histopathological items. STCDC-related effects were clearly observed in all groups, showing most damage at four hours, progressing to repair, and nearly normal at two weeks post-dose.

The relatively fast repopulation of respiratory and olfactory epithelial cells after treatment suggest that the loss of the epithelium may have been due in part to fragility of the intercellular connections. There was no evidence of ulceration, and an intact basement membrane may explain the relatively quick repopulation of cells.

This disclosure extends beyond the specifically disclosed embodiments and examples to other alternative embodiments and/or other uses of the embodiments, as well as to certain modifications and equivalents thereof. Moreover, this disclosure expressly contemplates that various features and aspects of the disclosed embodiments can be combined with, or substituted for, one another. Accordingly, the scope of this disclosure should not be limited by the particular disclosed embodiments described above, but should be determined only by a fair reading of the claims.

What is claimed:

1. A pharmaceutical formulation comprising:
a therapeutically effective amount of an active pharmaceutical ingredient (API);
an absorption enhancer consisting of one or more bile acids or bile acid salts at a concentration that is greater than 3 mg/ml; and an aqueous carrier;
wherein the pharmaceutical formulation is configured to be administered intranasally and/or via an intrapulmonary route;
wherein the pharmaceutical formulation is safe and effective for use in a subject, causing no irreversible damage to the subject.

2. The formulation of claim 1, wherein any irritation or adverse effects caused by administration of the pharmaceutical formulation are transient.

3. The formulation of claim 2, wherein irritation or adverse effects caused by administration of the pharmaceutical formulation resolve completely in less than or equal to 1 day, three days, one week, or two weeks.

4. The formulation of claim 1, wherein concentration of one or more bile acids or bile acid salts is provided at a concentration of equal to or less than 1.5 weight percent in the formulation.

5. The formulation of claim 1, wherein the one or more bile acids or bile acid salts is provided at a concentration above its critical micelle concentration (CMC).

6. The formulation of claim 1, wherein the formulation comprises micelles that include the one or more bile acids or bile acid salts.

7. The formulation of claim 6, wherein the micelles are configured to facilitate transcellular passage and enhance absorption of the API.

8. The formulation of claim 1, wherein the API comprises small drug molecules, large biologics, complex molecules, or combinations thereof.

9. The formulation of claim 1, wherein the absorption enhancer is configured to provide bioavailability of the API that is comparable to administration of the API through an intramuscular delivery route and/or wherein intranasal administration using the formulation may be used as a substitute for the intramuscular delivery route.

10. The formulation of claim 1, wherein the formulation comprises a therapeutically effective amount of API is suitable for the treatment of a type-I hypersensitivity reaction.

11. The formulation of claim 1, wherein the absorption enhancer consists of a taurocholate salt.

12. The formulation of claim 1, wherein the absorption enhancer consists of sodium taurocholate.

13. The formulation of claim 1, wherein the absorption enhancer consists of a taurochenodeoxycholate.

14. The formulation of claim 1, wherein the absorption enhancer consists of sodium taurochenodeoxycholate.

15. The formulation of claim 1, wherein the pharmaceutical formulation further comprises a buffer.

16. The formulation of claim 1, wherein the pharmaceutical formulation further comprises a preservative.

17. The formulation of claim 1, wherein the pharmaceutical formulation further comprises a tonicity agent.

18. The formulation of claim 1, wherein the pharmaceutical formulation further comprises a metal complexing agent.

19. The formulation of claim 1, wherein the pharmaceutical formulation further comprises an antioxidant.

20. The formulation of claim 1, wherein the pharmaceutical formulation has an osmolarity ranging from 200 mOsmol to 260 mOsmol.

21.-33. (canceled)
